(19) United States
(12) Patent Application Publication
(10) Pub. No.: US 2003/0224368 A1
(43) Pub. Date: Dec. 4, 2003

(54) Crystallizable compositions comprising a caspase-7

(76) Inventor: Yunyi Wei, Belmont, MA (US)

Correspondence Address: FISH & NEAVE, 1251 AVENUE OF THE AMERICAS, 50TH FLOOR, NEW YORK, NY, 10020-1105, US

(21) Appl. No.: 10/151,119

(22) Filed: May 16, 2002

Related U.S. Application Data
(63) Continuation of application No. PCT/US00/31602, filed on Nov. 16, 2000.
(60) Provisional application No. 60/165,797, filed on Nov. 16, 1999.

(51) Int. Cl. C12Q 1/68

(52) U.S. Cl. 435/6

(57) ABSTRACT

The present invention relates to a data storage medium encoded with the structural coordinates of crystallized molecules and molecular complexes which comprise the active site binding pockets of caspase-7. Such data storage material is capable of displaying such molecules and molecular complexes, or their structural homologues, as a graphical three-dimensional representation on a computer screen. This invention also related to methods of using the structure coordinates to solve the structure of similar or homologous proteins or protein complexes. In addition, this invention relates to methods of using the structure coordinates to screen and design compounds, including inhibitory compounds, that bind to caspase-7 or homologues thereof. This invention also relates to molecules or molecular complexes which comprise the active site binding pockets of caspase-7 or close structural homologues of the active site binding pockets. The present invention also relates to compositions and crystals of a caspase-7 in complex with a caspase inhibitor. This invention also relates to compounds and pharmaceutical compositions which are inhibitors of caspase-7.

### TECHNICAL FIELD OF INVENTION

The present invention relates to a data storage medium encoded with the structural coordinates of crystallized molecules and molecular complexes which comprise the active site binding pockets of caspase-7. Such data storage material is capable of displaying such molecules and molecular complexes, or their structural homologues, as a graphical three-dimensional representation on a computer screen. This invention also related to methods of using the structure coordinates to solve the structure of similar or homologous proteins or protein complexes. In addition, this invention relates to methods of using the structure coordinates to screen and design compounds, including inhibitory compounds, that bind to caspase-7 or homologues thereof. This invention also relates to molecules or molecular complexes which comprise the active site binding pockets of caspase-7 or close structural homologues of the active site binding pockets. The present invention also relates to compositions and crystals of a caspase-7 in complex with a caspase inhibitor.

### BACKGROUND OF THE INVENTION

Apoptosis, or programmed cell death, is a principal mechanism by which organisms eliminate unwanted cells. The deregulation of apoptosis, either excessive apoptosis or the failure to undergo it, has been implicated in a number of diseases such as cancer, acute inflammatory and autoimmune disorders, ischemic diseases and certain neurodegenerative disorders [see generally Science, 281, pp. 1283-1312 (1998); Ellis et al., Ann. Rev. Cell. Biol., 7, p. 663 (1991)].

Caspases are a family of cysteine protease enzymes that are key mediators in the signaling pathways for apoptosis and cell disassembly [N.A. Thornberry, Chem. Biol., 5, pp. R97-R103 (1998)]. These signaling pathways vary depending on cell type and stimulus, but all apoptosis pathways appear to converge at a common effector pathway leading to proteolysis of key proteins. Caspases are involved in both the effector phase of the signaling pathway and further upstream at its initiation. The upstream caspases involved in initiation events become activated and in turn activate other caspases that are involved in the later phases of apoptosis.

The caspases have been classified into three groups depending on their predominant functional roles and their substrate specificities [N. A. Thornberry, Chem. Biol., 5, pp. R97-R103 (1998); N. A. Thornberry & Y. Lazebnik, Science, 281, pp. 1312-1316 (1998); M. Garcia-Calvo et al., J. Biol. Chem., 273, pp. 32608-32613 (1998)].

The first subfamily consists of caspases-1 (ICE), 4, and S. These caspases have been shown to be involved in pro-inflammatory cytokine processing and therefore play an important role in inflammation. Caspase-1, the most studied enzyme of this class, activates the IL-1β precursor by proteolytic cleavage. This enzyme therefore plays a key role in the inflammatory response. Caspase-1 is also involved in the processing of interferon gamma inducing factor (IGIF or IL-18) which stimulates the production of interferon gamma, a key immunoregulator that modulates antigen presentation, T-cell activation and cell adhesion.

The remaining caspases make up the second and third subfamilies. These enzymes are of central importance in the intracellular signaling pathways leading to apoptosis. One subfamily consists of the enzymes involved in initiating events in the apoptotic pathway, including transduction of signals from the plasma membrane. Members of this subfamily include caspases-2, 8, 9, and 10. The other subfamily, consisting of the effector caspases 3, 6, and 7, are involved in the final downstream cleavage events that result in the systematic breakdown and death of the cell by apoptosis. Caspases involved in the upstream signal transduction activate the downstream caspases, which then disable DNA repair mechanisms, fragment DNA, dismantle the cell cytoskeleton and finally fragment the cell.

The utility of caspase inhibitors to treat a variety of mammalian disease states associated with an increase in cellular apoptosis has been demonstrated using peptidic caspase inhibitors. For example, in rodent models, caspase inhibitors have been shown to reduce infarct size and inhibit cardiomyocyte apoptosis after myocardial infarction, to reduce lesion volume and neurological deficit resulting from stroke, to reduce post-traumatic apoptosis and neurological deficit in traumatic brain injury, to be effective in treating fulminant liver destruction, and to improve survival after endotoxic shock [H. Yaoita et al., Circulation, 97, pp. 276-281 (1998); M. Endres et al., J. Cerebral Blood Flow and Metabolism, 18, pp. 238-247, (1998); Y. Cheng et al., J. Clin. Invest., 101, pp. 1992-1999 (1998); A. G. Yakovlev et al., J. Neurosci., 17, pp. 7415-7424 (1997); I. Rodriquez et al., J. Exp. Med., 184, pp. 2067-2072 (1996); Grobmyer et al., Mol. Med., 5, p. 585 (1999)].

Caspase-7 is considered a potential target for therapeutic agents. The current understanding of caspase-7 has not however led to satisfactory treatments for caspase-7 mediated disease. Thus, there is a need for more effective caspase-7 inhibitors. There is also a need for inhibitors that either inhibit caspase-7 selectively or inhibit caspase-7 as well as other caspases.

Drug discovery efforts directed towards caspase-7 have been hampered by the lack of structural information about caspase-7. Such structural information would be valuable in the discovery of selective caspase-7 inhibitors and pan-caspase inhibitors. However, efforts to determine the structure of caspase-7 have been hampered by difficulties in crystallizing caspase-7. There have been no crystals reported of a caspase-7 protein. Thus, x-ray crystallographic analysis of such proteins has not been possible.

### SUMMARY OF THE INVENTION

Applicants have solved this problem by providing, for the first time, the crystallization of a caspase-7 in complex with a caspase-7 inhibitor and the structure coordinates of that complex. Solving the three-dimensional crystal structure of that complex has allowed applicants to determine key structural features of caspase-7, particularly the shape of its active site binding pockets.

Thus, the present invention provides molecules or molecular complexes that comprise all or parts of these binding pockets, or homologues of these binding pockets that have similar three-dimensional shapes.

The present invention relates to a data storage medium that comprises the structural coordinates of crystallized molecules and molecular complexes which comprise caspase-7, including all or any parts of the caspase-7 active site binding pockets. The data storage medium is capable of displaying the molecules and molecular complexes, or their structural homologues, as a graphical three-dimensional representation on a computer screen.

The invention also provides a method for determining at least a portion of the three-dimensional structure of molecules or molecular complexes which contain at least some structurally similar features to a caspase-7. This is achieved by using at least some of the structure coordinates obtained for caspase-7.

In addition, this invention relates to methods of using the structure coordinates to screen and design compounds, including inhibitory compounds, that bind to caspase-7 or homologues thereof.

The invention also provides crystallizable compositions and crystals of a caspase-7/inhibitor complex and methods for making such crystals.

### BRIEF DESCRIPTION OF THE FIGURES

FIGS. 1A and 1B depict a stereoview of a caspase-7 tetrameric assembly made using RIBBONS [M. Carson, J. Appl. Cryst., 24, pp. 958-961 (1991)]. The p20, p10 and their symmetry related equivalents, p20′ and p10′, are shown moving from left to right in each of FIG. 1A and FIG. 1B. The ball-and-stick model, near the top left of each FIG., represents the Ac-DEVD-CHO inhibitor.

FIGS. 2A, 2B, and 2C depict secondary structural elements of caspase-1, caspase-3, caspase-7, and caspase-8.

FIG. 2A depicts the conserved fold for superimposed Caspase-l, Caspase-3, Caspase-7, Caspase-8 with covalently bound tetrapeptide inhibitor, Ac-DEVD-CHO.

FIG. 2B depicts superimposed S4 loops for caspase-1 (378-386), caspase-3 (244-262), caspase-7 (270-288), and caspase-8 (451-463).

FIG. 2C depicts prime side helix-turn-helix insertion in caspase-8 ranging from residues 245-253 proximal to superimposed insertion loop of caspase-1 (residues 249-254). FIG. 2B and FIG. 2C have been rotated slightly relative to FIG. 2A.

FIG. 3 depicts the sequence alignment of caspase-1, caspase-3, caspase-7, and caspase-8. The alignment was heavily biased upon the superposition of conserved secondary structural elements and active site residues of caspase-1, caspase-3, caspase-7, and caspase-8. The alignment was performed with the MVP program and then adjusted manually [M. H. Lambert, Pract. Appl. Comput.-Aided Drug Des., pp. 243-303, P.S. Charifson, ed., Dekker, N.Y. (1997)]. Boxed residues denote direct or water-mediated interactions with Ac-DEVD-CHO as shown in FIG. 4.

FIG. 4 depicts hydrogen bonding and van der Waals interactions of covalently bound Ac-DEVD-thiohemiacetal with binding site residues of: a) caspase-1; b) caspase-3; c) caspase-7; and d) caspase-8. “Wat” indicates a water molecule.

FIGS. 5A-5D depicts electrostatic potentials mapped onto molecular surface for binding site residues of caspase-1, caspase-3, caspase-7, and caspase-8. Areas of positive electrostatic potential, negative electrostatic potential, and neutral electrostatic potential are depicted. The molecular surface and electrostatic potentials calculated with GRASP [A. Nicholls et al., Proteins: Structure Function, Genetics, 11, pp. 281-296 (1991)).

FIG. 6 depicts a stereoview diagram showing the superposition of caspase-3 with caspase-7 generated by RIBBONS. The N-acetyl group of the tetrapeptide inhibitor, Ac-DEVD-CHO, bound to caspase-7 is translated approximately 2.5 Å relative to that in caspase-3 due to the substitution of proline (235) for serine at the same position of the P4-binding site.

FIG. 7 lists the atomic structure coordinates for caspase-7 in complex with a synthetic tetrapeptide inhibitor as derived by X-ray diffraction from a crystal of that complex. The preparation of the complex is described in Examples 1 and 2. The following abbreviations are used in FIG. 7:

“Atom type” refers to the element whose coordinates have been determined. Elements are defined by the first letter in the column.

“X, Y, Z” crystallographically define the atomic position determined for each atom.

“Occ” is an occupancy factor that refers to the fraction of the molecules in which each atom occupies the position specified by the coordinates. A value of “1” indicates that each atom has the same conformation, i.e., the same position, in all molecules of the crystal.

“B” is a thermal factor that measures movement of the atom around its atomic center.

FIG. 8 shows a diagram of a system used to carry out the instructions encoded by the storage medium of FIGS. 9 and 10.

FIG. 9 shows a cross section of a magnetic storage medium.

FIG. 10 shows a cross section of an optically-readable data storage medium.

### DETAILED DESCRIPTION OF THE INVENTION

The following abbreviations are used throughout the application:

| | |
|---|---|
| A = Ala = Alanine | T = Thr = Threonine |
| V = Val = Valine | C = Cys = Cysteine |
| L = Leu = Leucine | Y = Tyr = Tyrosine |
| I = Ile = Isoleucine | N = Asn = Asparagine |
| P = Pro = Proline | Q = Gln = Glutamine |
| F = Phe = Phenylalanine | D = Asp = Aspartic Acid |
| W = Trp = Tryptophan | E = Glu = Glutamic Acid |
| M = Met = Methionine | K = Lys = Lysine |
| G = Gly = Glycine | R = Arg = Arginine |
| S = Ser = Serine | H = His = Histidine |

Additional definitions are set forth in the specification where necessary.

In order that the invention described herein may be more fully understood, the following detailed description is set forth.

Applicants have solved the above problems by providing, for the first time, crystallizable compositions comprising a caspase-7 in complex with a caspase-7 inhibitor.

Thus, in one embodiment of this invention is provided a crystallizable composition comprising a caspase-7 in complex with a tetrapeptide aldehyde inhibitor, acetyl-Asp-Glu-Val-Asp-CHO (Ac-DEVD-CHO). Preferably, the caspase-7 has amino acids 1-303 according to Y. Gu et al., J. Biol. Chem., 271, pp. 10816-10820 (1996).

The caspase-7 polypeptide portion of the complex is any polypeptide which has the cysteine protease activity of the naturally occurring caspase-7.

Preferably, the caspase-7 polypeptide in the compositions of this invention is the recombinantly produced caspase-7 protein that is prepared as described herein.

The caspase-7 polypeptide and Ac-DEVD-CHO may be produced by any well-known method, including synthetic methods, such as solid phase, liquid phase and combination solid phase/liquid phase syntheses; recombinant DNA methods, including cDNA cloning, optionally combined with site directed mutagenesis; and/or purification of the natural products, optionally combined with enzymatic cleavage methods to produce fragments of naturally occurring caspase-7 polypeptide. The inhibitor Ac-DEVD-CHO is commercially available (Peptides International) or may be produced by any well-known method, including synthetic methods, such as solid phase, liquid phase and combination solid phase/liquid phase syntheses.

In another embodiment of this invention is provided a crystal comprising a caspase-7 in complex with a tetrapeptide aldehyde inhibitor, acetyl-Asp-Glu-Val-Asp-CHO (Ac-DEVD-CHO). Preferably, the caspase-7 has amino acids 1-303 according to Y. Gu et al., J. Biol. Chem., 271, pp. 10816-10820 (1996). Preferably, the crystal has unit cell dimensions of 88.2 Å, 88.2 Å, 186.2 Å, α=90.0°, β=90.0°, γ=120.0° and belongs to space group P₃221. More preferably, the crystallized enzyme is a tetramer.

Importantly, applicants' invention has provided, for the first time, information about the shape and structure of the caspase-7 active site binding pockets.

Binding pockets are of significant utility in fields such as drug discovery. The association of natural ligands or substrates with the binding pockets of their corresponding receptors or enzymes is the basis of many biological mechanisms of action. Similarly, many drugs exert their biological effects through association with the binding pockets of receptors and enzymes. Such associations may occur with all or any parts of the binding pocket. An understanding of such associations will help lead to the design of drugs having more favorable associations with their target receptor or enzyme, and thus, improved biological effects. Therefore, this information is valuable in designing potential inhibitors of caspase-7-like binding pockets.

The term “binding pocket”, as used herein, refers to a region of a molecule or molecular complex, that, as a result of its shape, favorably associates with another chemical entity or compound.

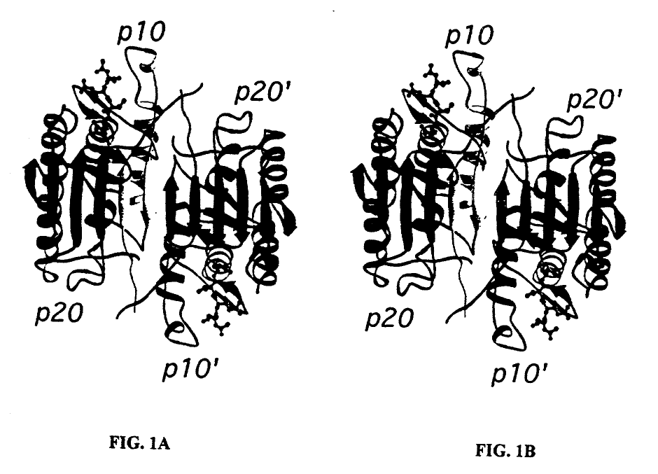

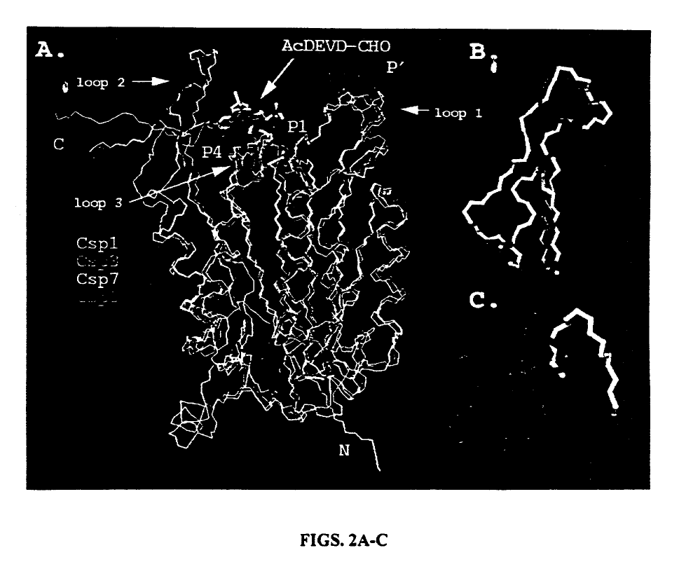

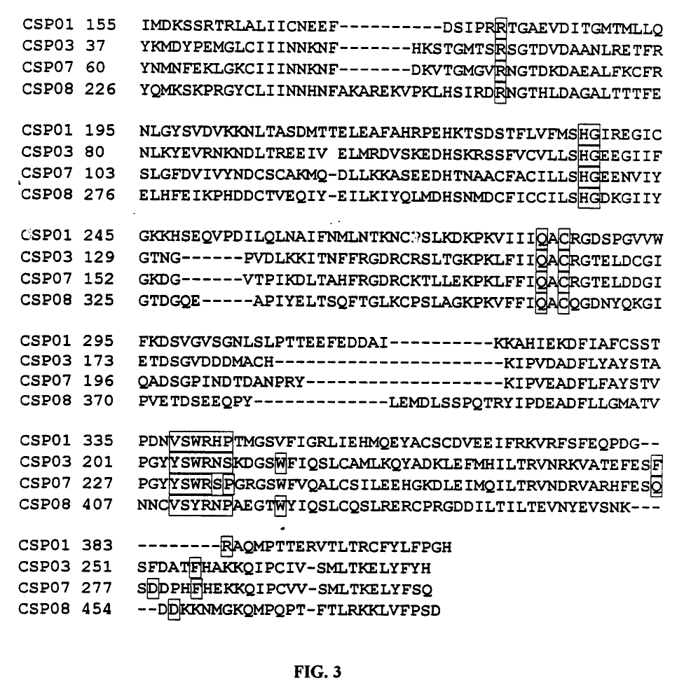

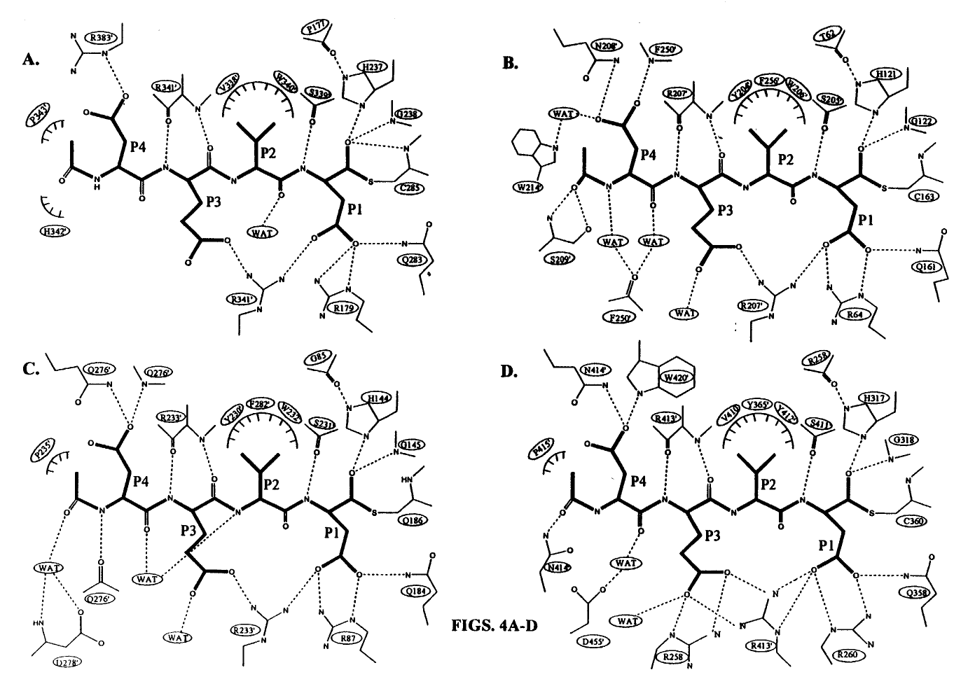

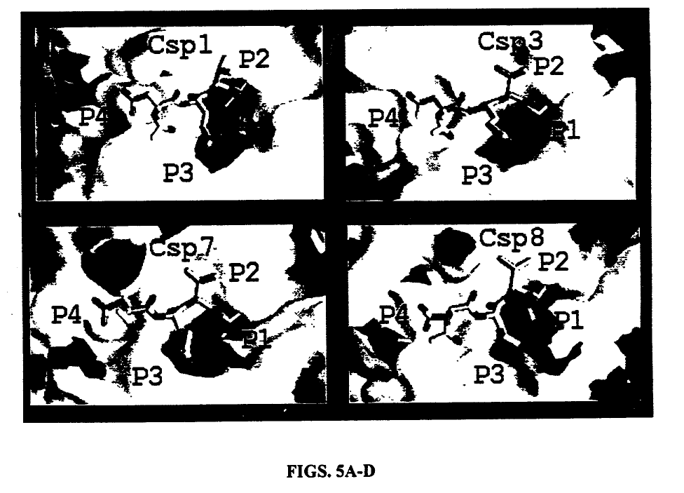

The term “caspase-7-like binding pocket” refers to a portion of a molecule or molecular complex whose shape is sufficiently similar to all or any parts of the caspse-7 active site binding pockets as to bind common ligands. This commonality of shape is defined by a root mean square deviation from the structure coordinates of the backbone atoms of the amino acids that make up the binding pockets in caspse-7 (as set forth in FIG. 1) of not more than 1.5 Å. How this calculation is obtained is described below.

The “active site binding pockets” or “active site” of caspse-7 refers to the area on the caspse-7 enzyme surface where cleavage of a substrate occurs, and where Ac-DEVD-CHO exerts its inhibitory effect.

The terms “P binding pocket”, “S pocket” “S region” and the like, refer to binding subsites, or portions of the substrate binding site on the caspase molecule. The amino acid residues of the substrate are given designations according to their position relative to the scissile bond, i.e., the bond that is broken by the protease. The residues are designated P1, P2, etc., for those extending toward the N-terminus of the substrate and P1′, P2′, etc., for those extending toward the C-terminus of the substrate. The portions of an inhibitor that correspond to the P or P′ residues of the substrate are also labeled P1, P1′, etc., by analogy with the substrate. The binding subsites of the caspase molecule that receive the residues labeled P1, P1′, etc., are designated S1, S1′, etc., or may alternately be designated “the P1 binding pocket”, “the P1′ binding pocket”, etc. [I. Schechter & A. Berger, Biochem. Biophys. Res. Communi., 27, pp. 157-162 (1967)].

In resolving the crystal structure of caspase-7, applicants have determined that caspase-7 amino acids 234, 235, 237, 276, 278, 281, and 284 are responsible for the S4-binding region of caspase-7 being more hydrophilic than the S4-binding region of caspase-3. Applicants have also determined that amino acids 85, 86, 87, 88, 144, 145, 184, 186, 191, 223, 230, 231, 232, 233, 234, 235, 237, 240, 276, 278, 281, 282, and 284 are situated close enough to the Ac-DEVD-CHO inhibitor to interact with this ligand.

Applicants have also determined that amino acid residues 87, 184, and 233 are important in the P1 binding pocket of caspase-7; that amino acid residues 191, 230, 232, and 282 are important in the P2 binding pocket of caspase-7; that amino acid residues 86, 88, 233 are important in the P3 binding pocket of caspase-7; and that amino acid residues 234, 235, 237, 240, and 276 are important in the P3 binding pocket of caspase-7.

It will be readily apparent to those of skill in the art that the numbering of amino acids in other isoforms of caspase-7 may be different than that described herein.

Advantageously, the crystal provided by this invention is amenable to x-ray crystallography. Thus, this invention also provides the three-dimensional structure of a caspse-7 complex, specifically a caspase-7/Ac-DEVD-CHO complex, at 2.35 Å resolution. Importantly, this has provided for the first time, information about the shape and structure of caspase-7.

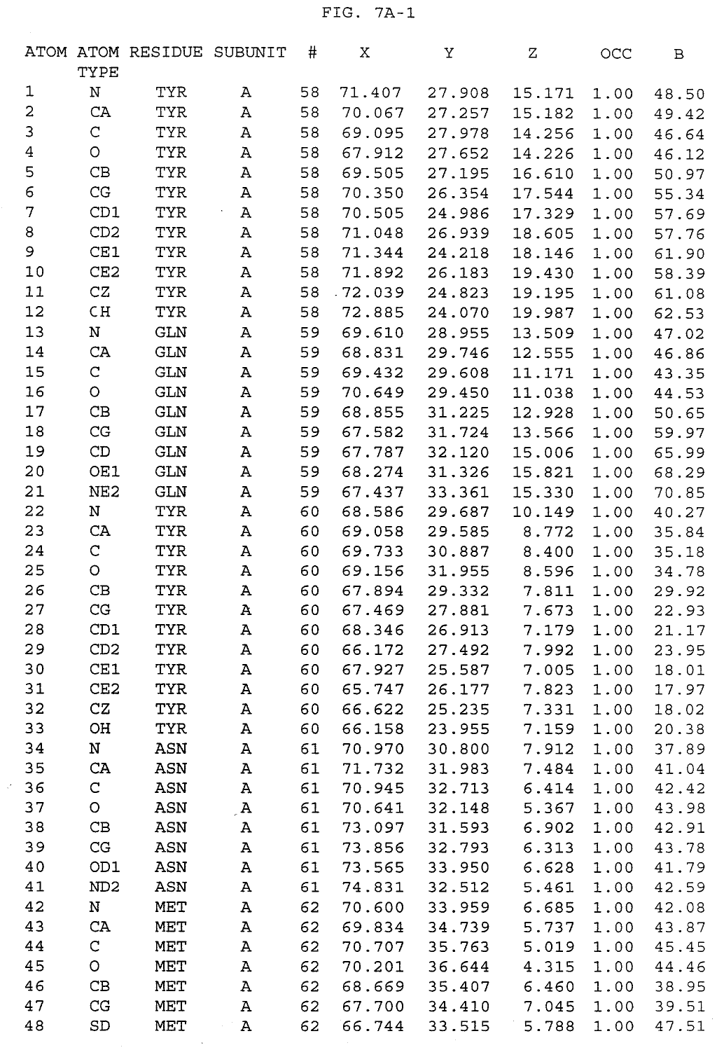

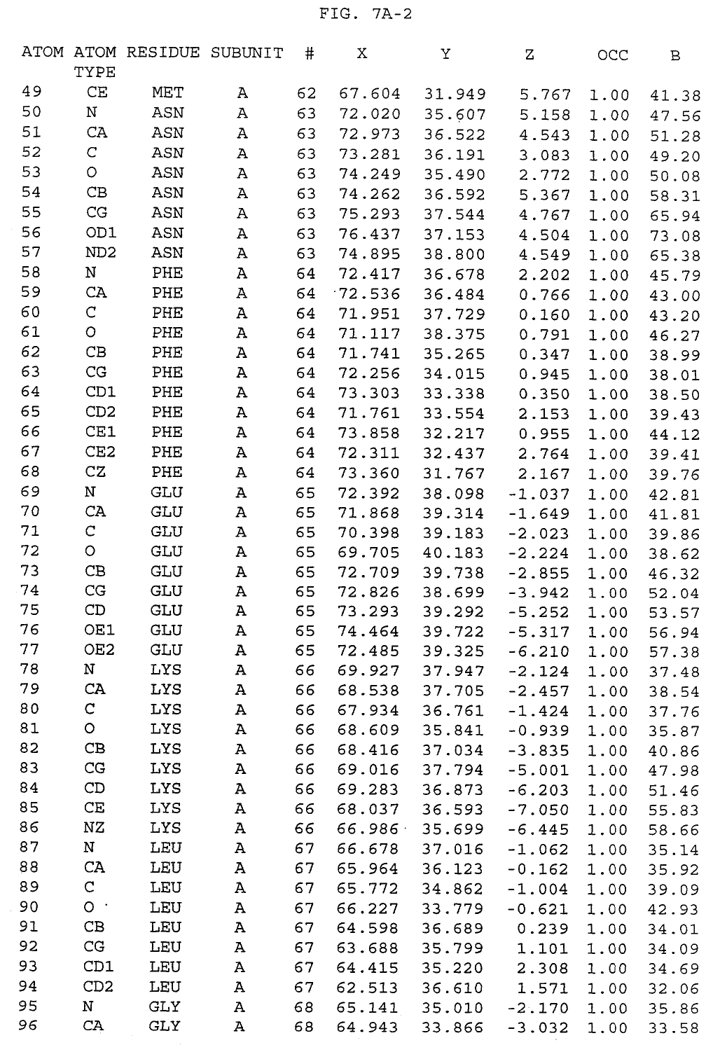

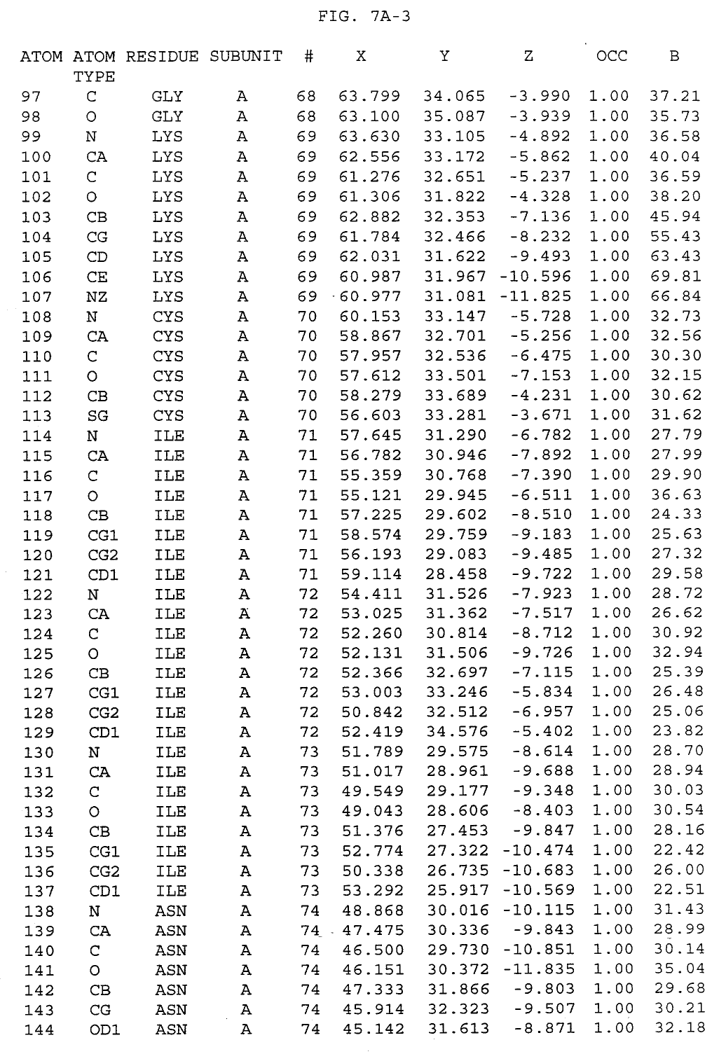

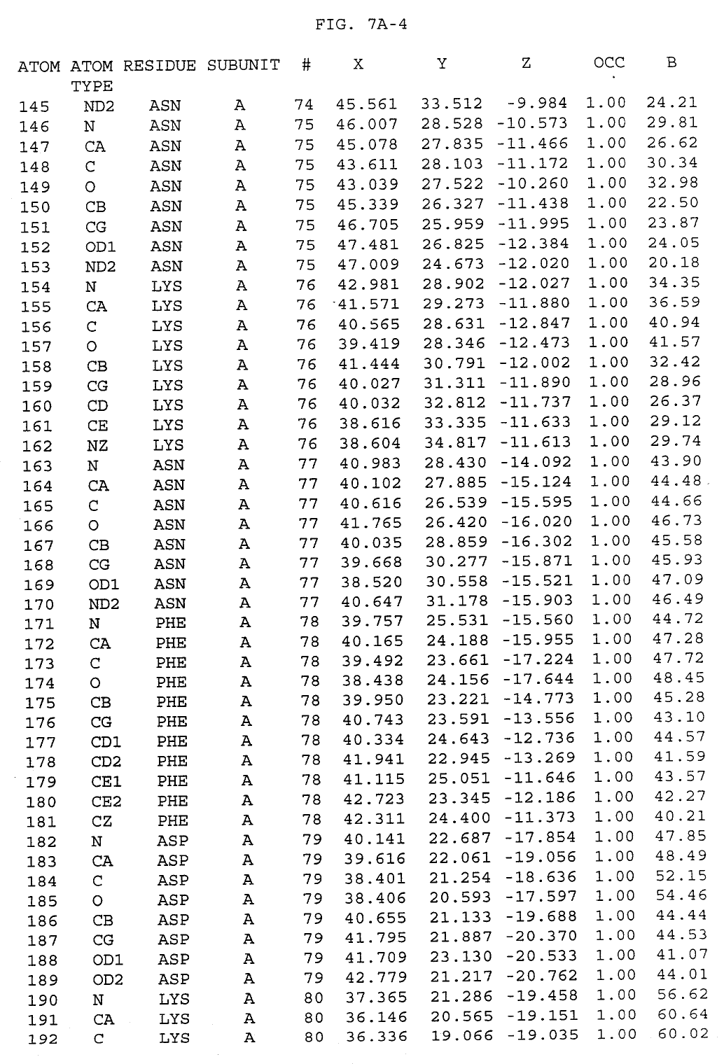

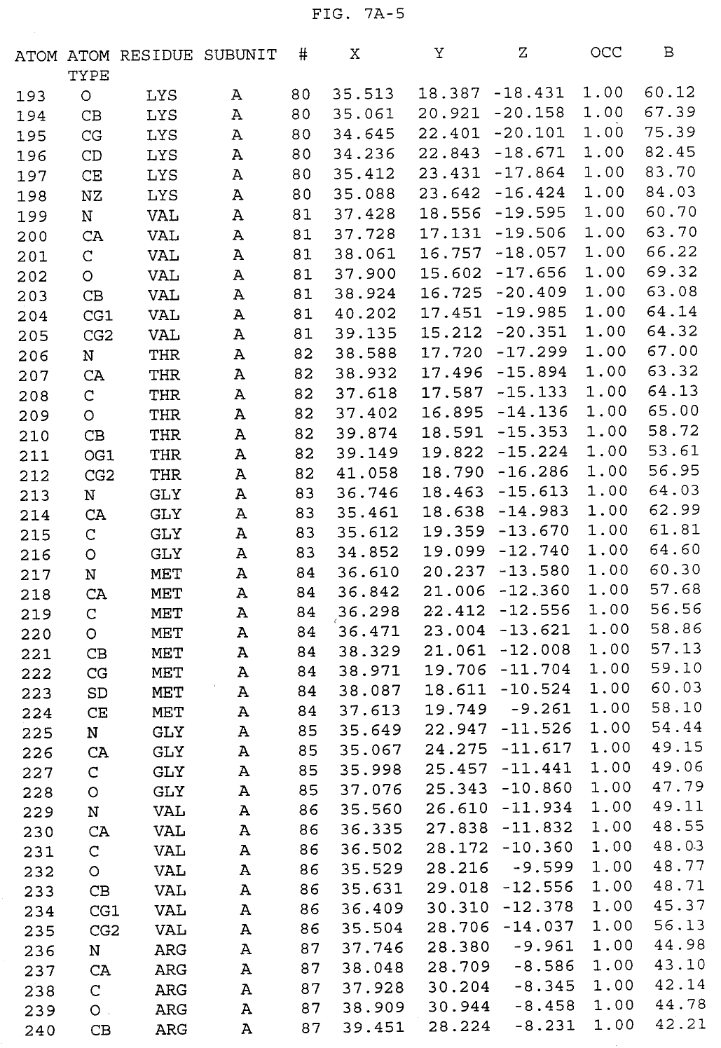

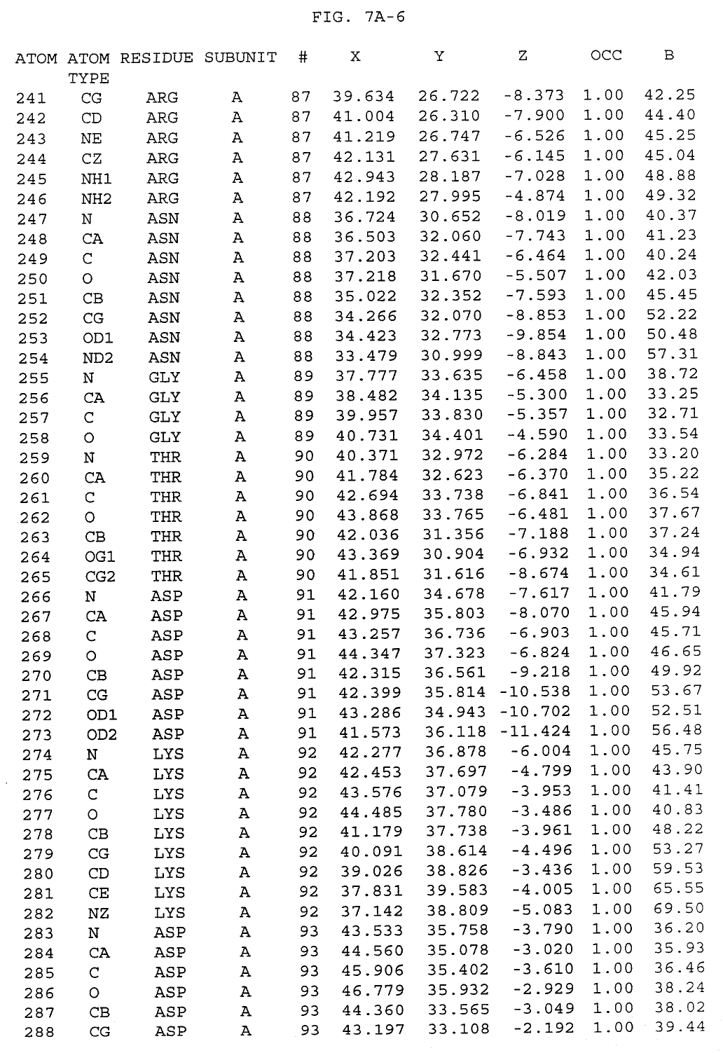

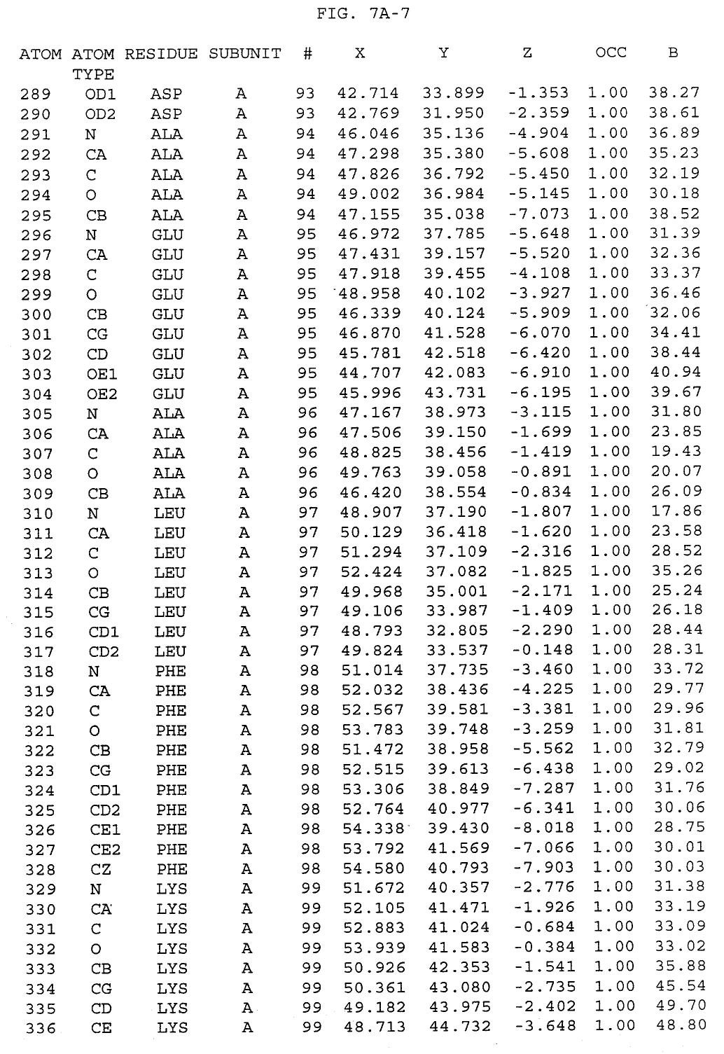

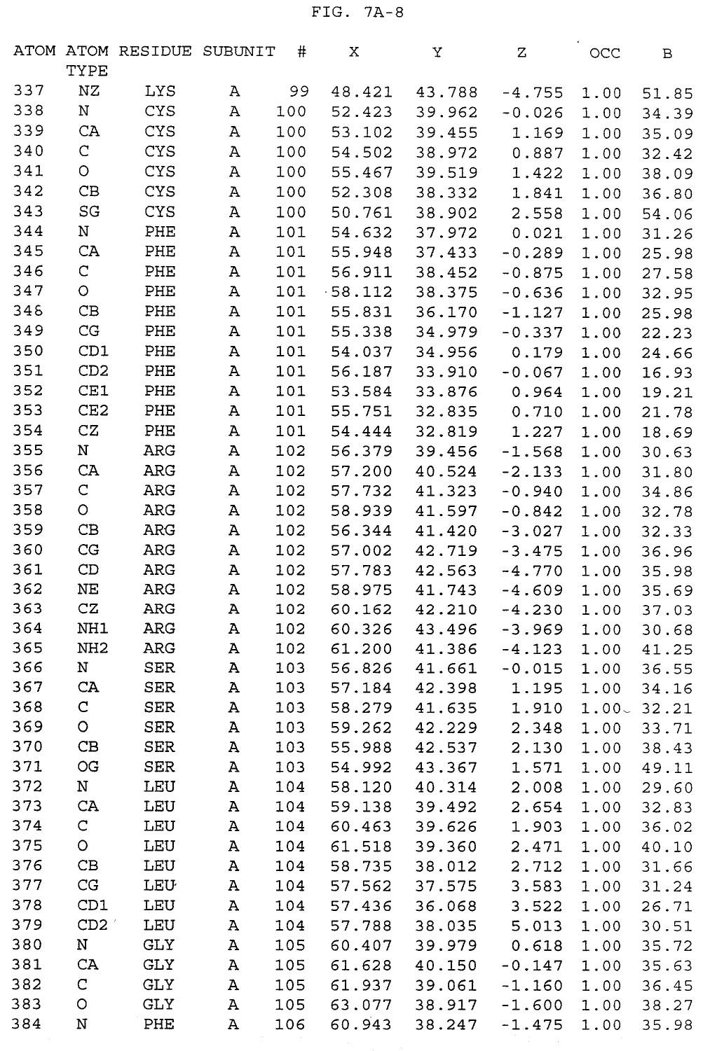

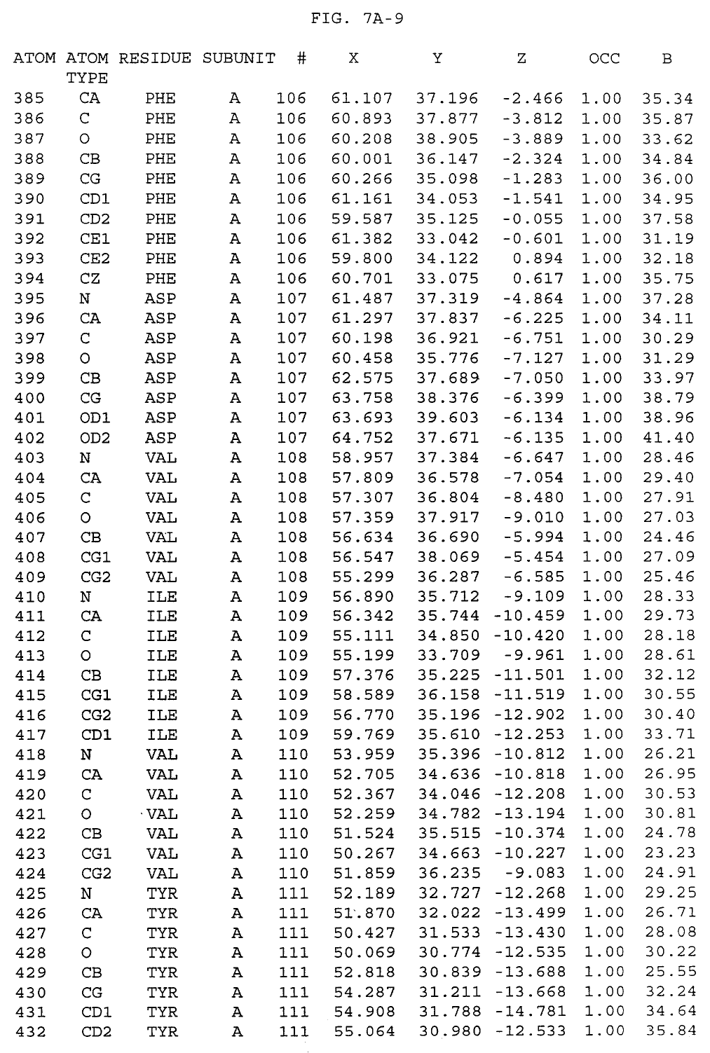

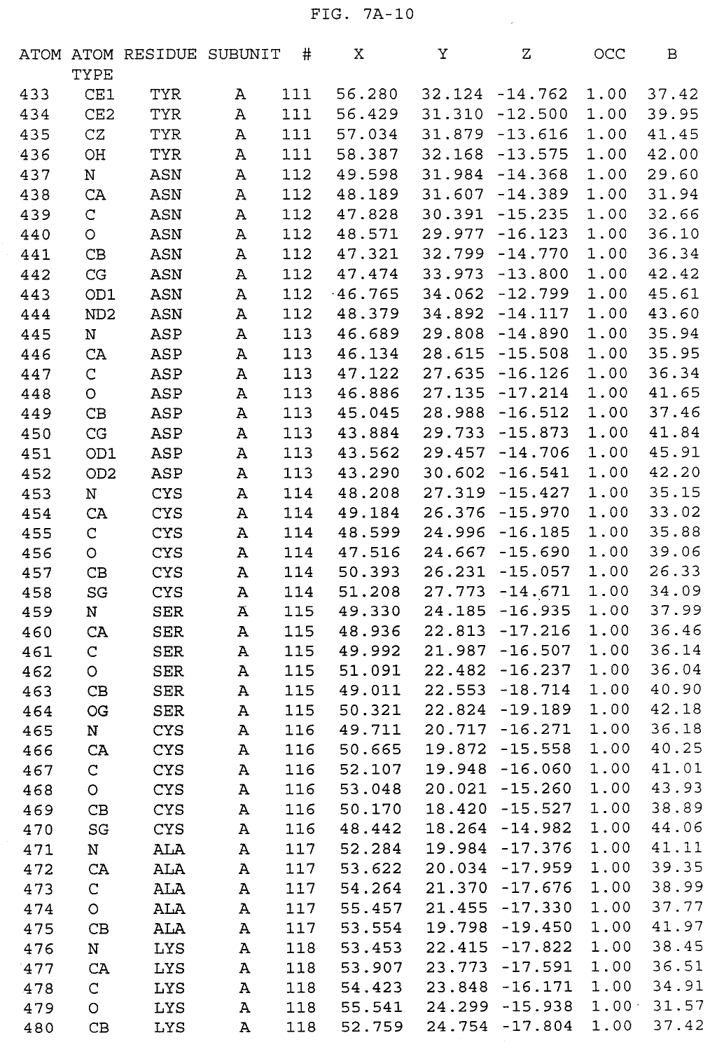

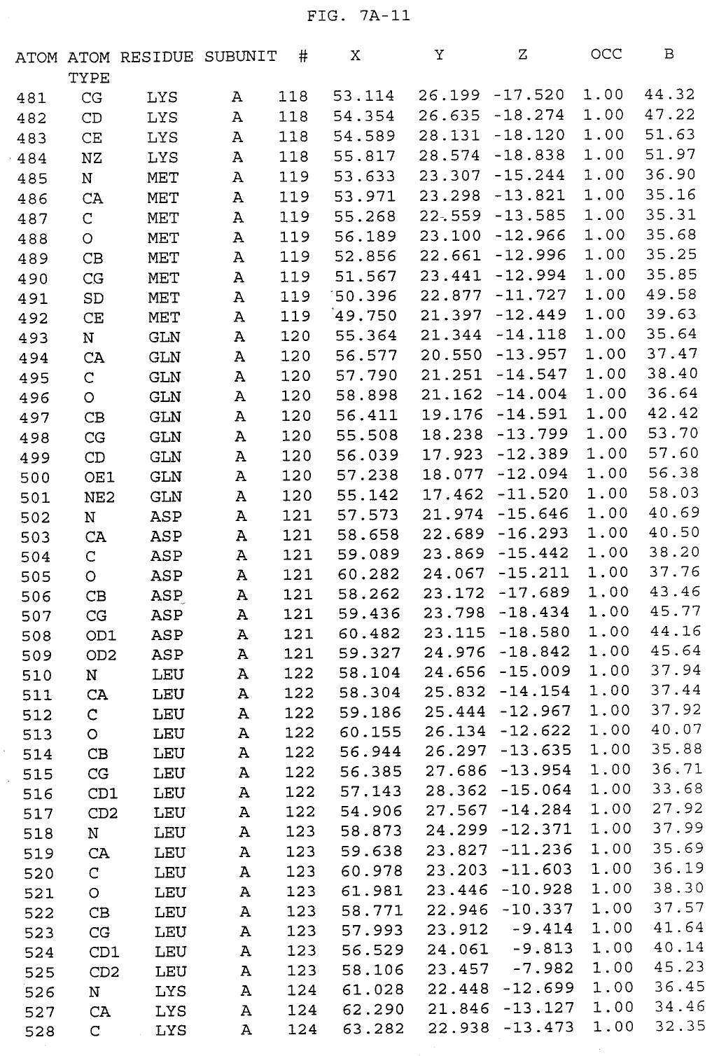

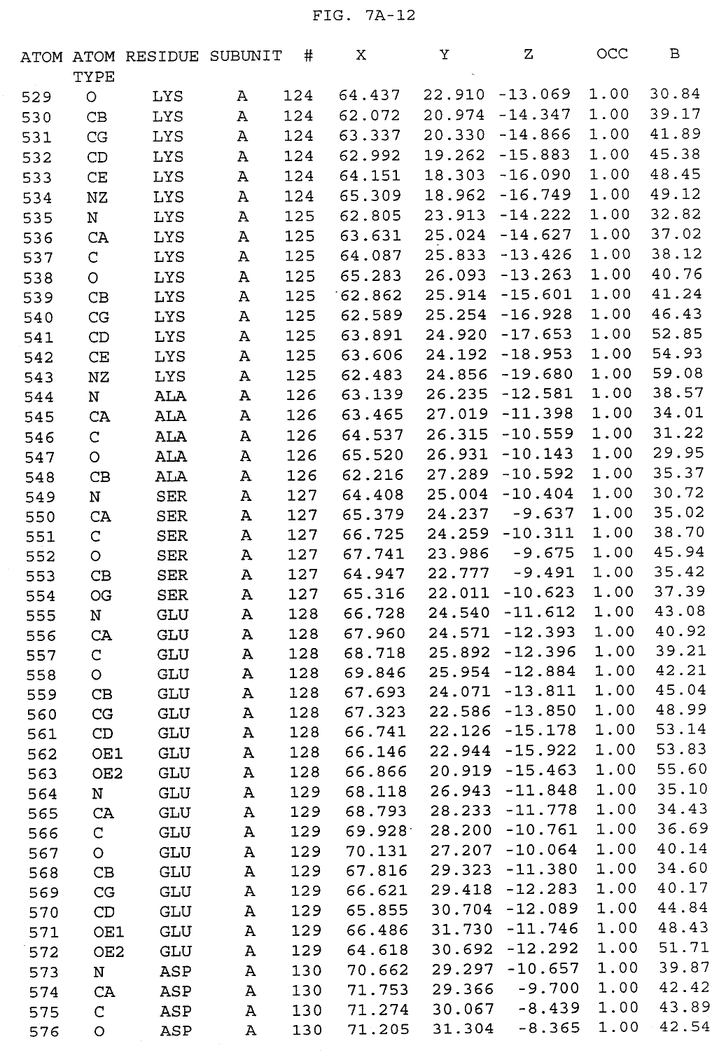

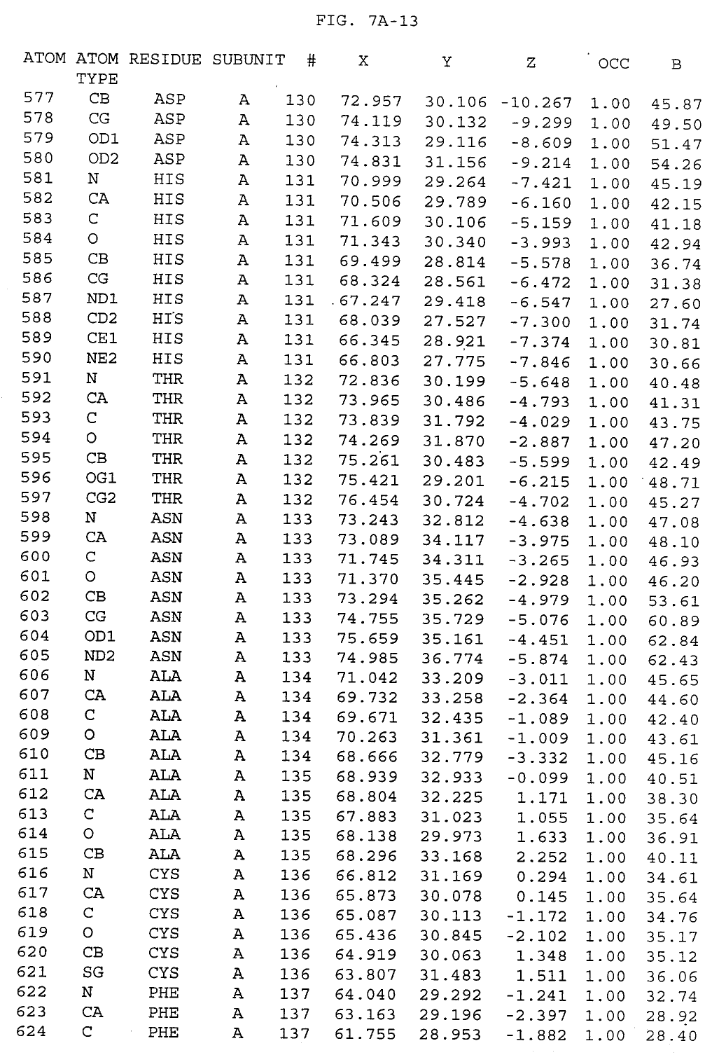

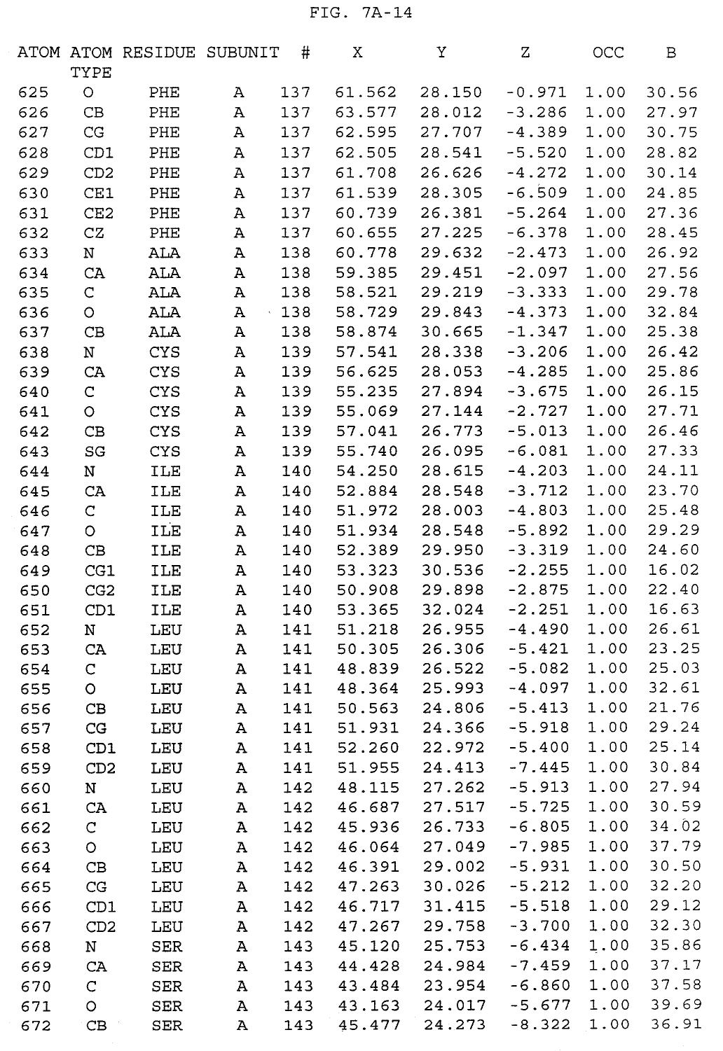

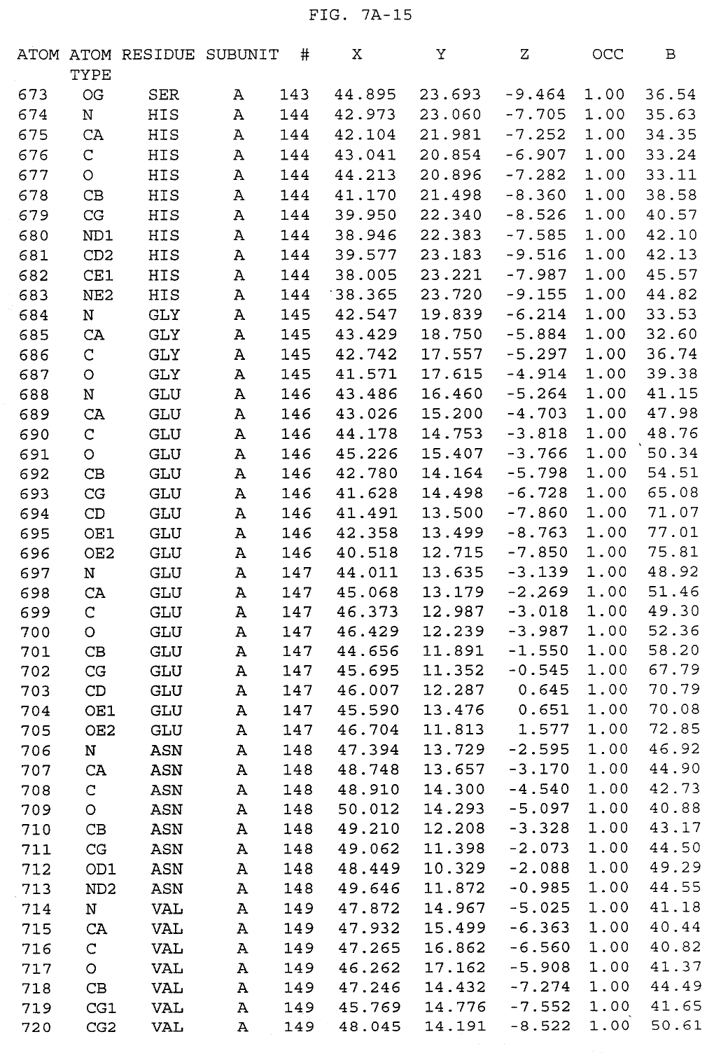

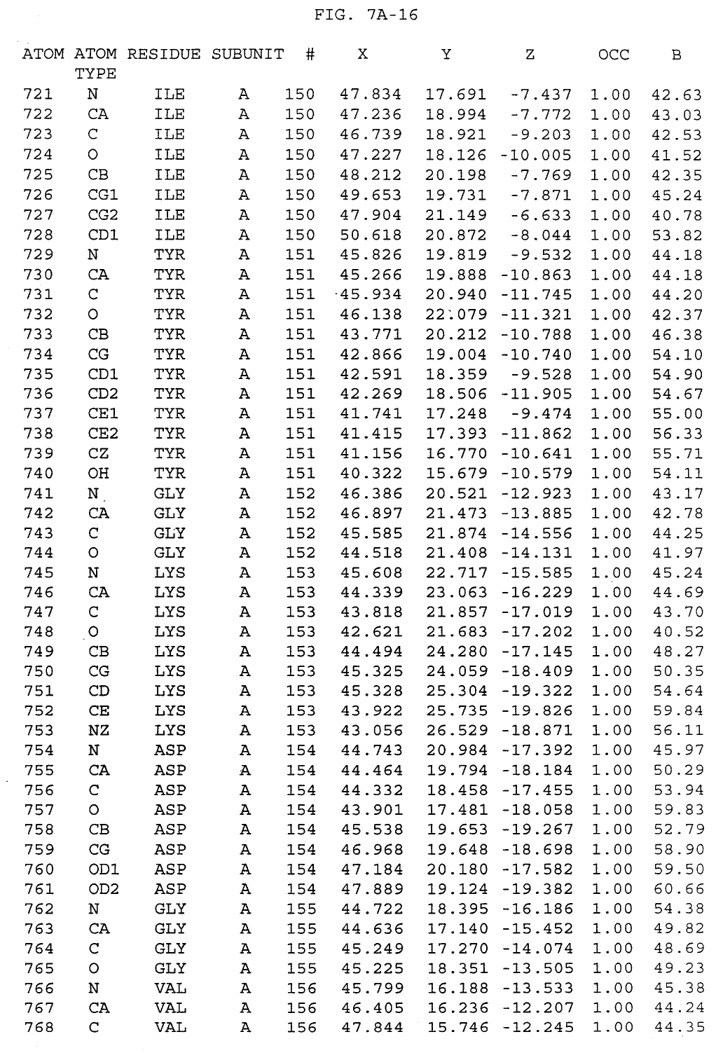

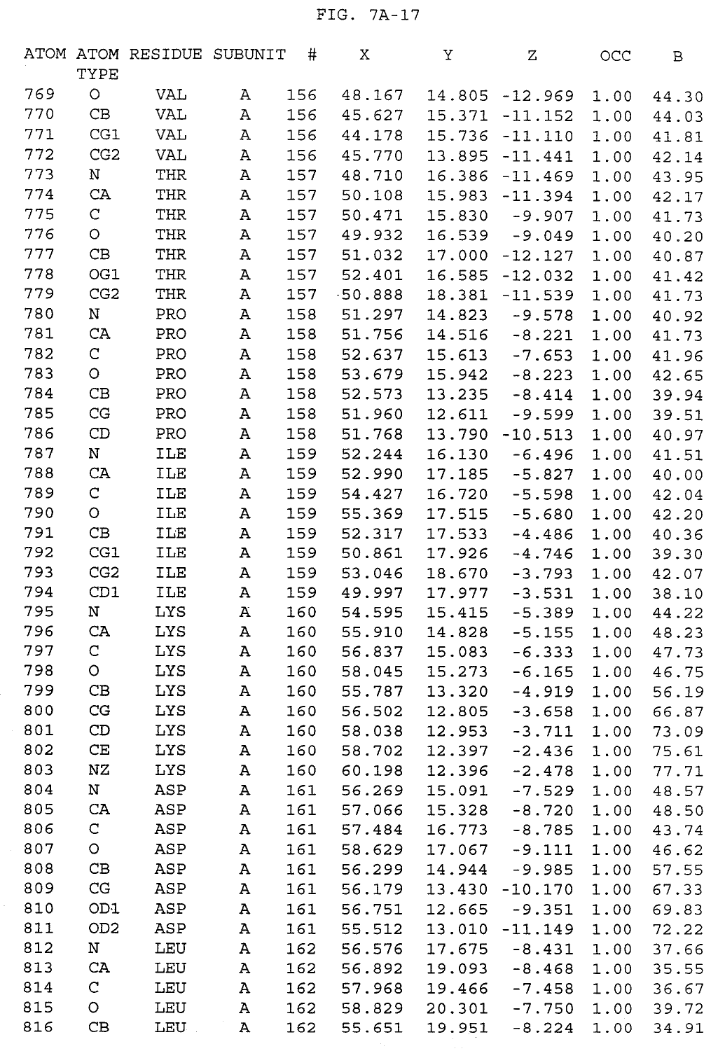

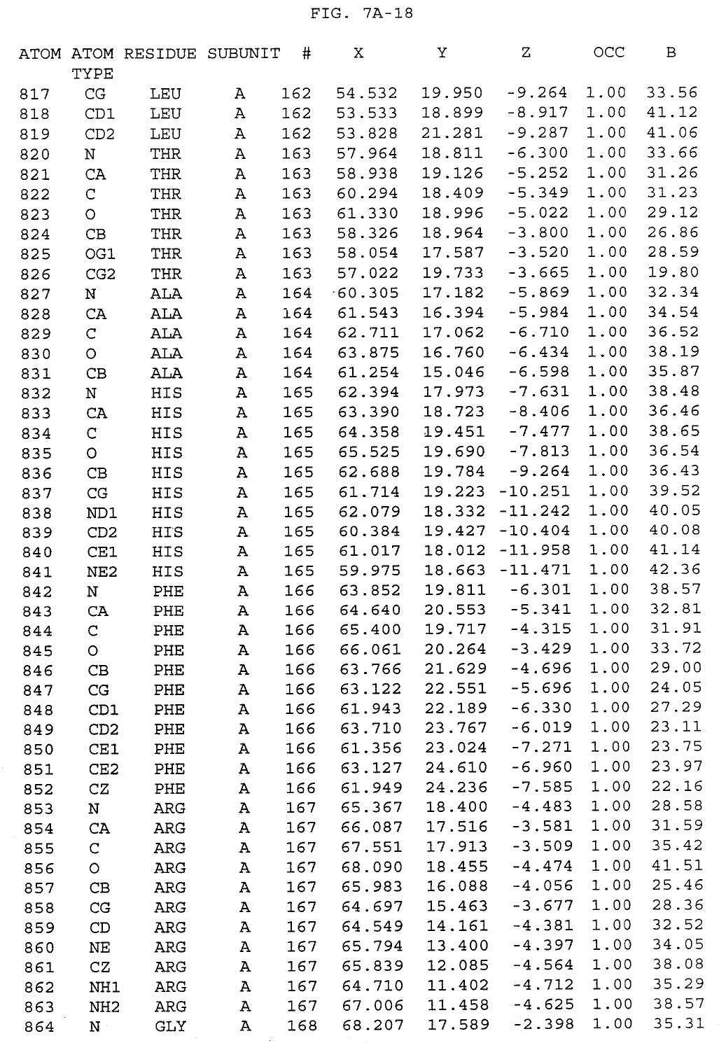

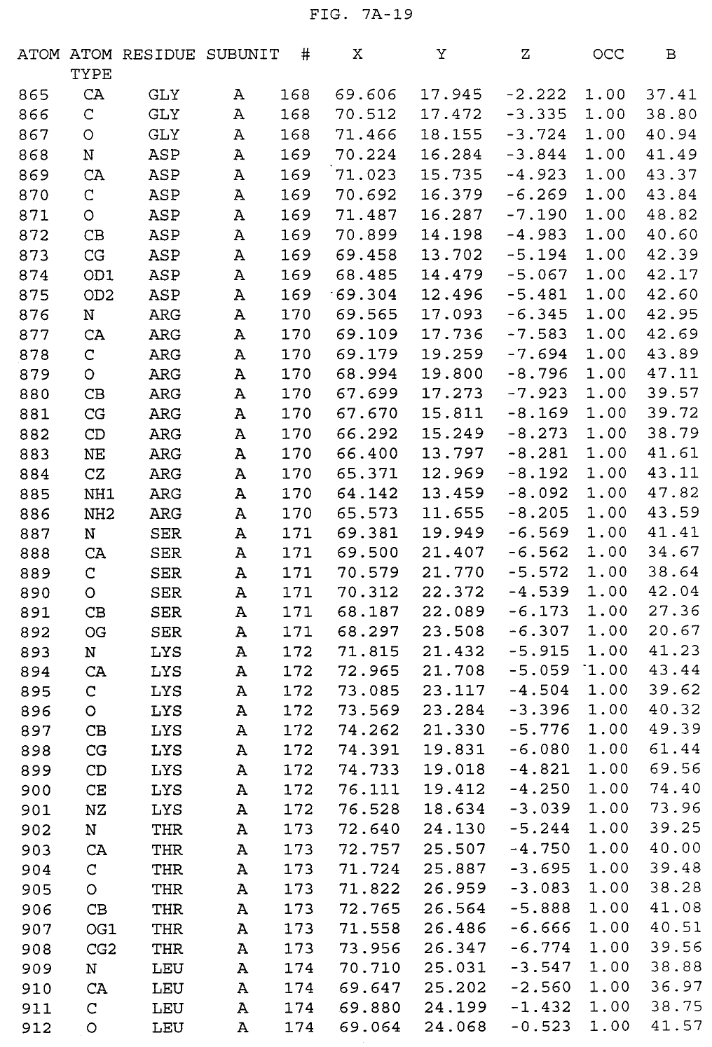

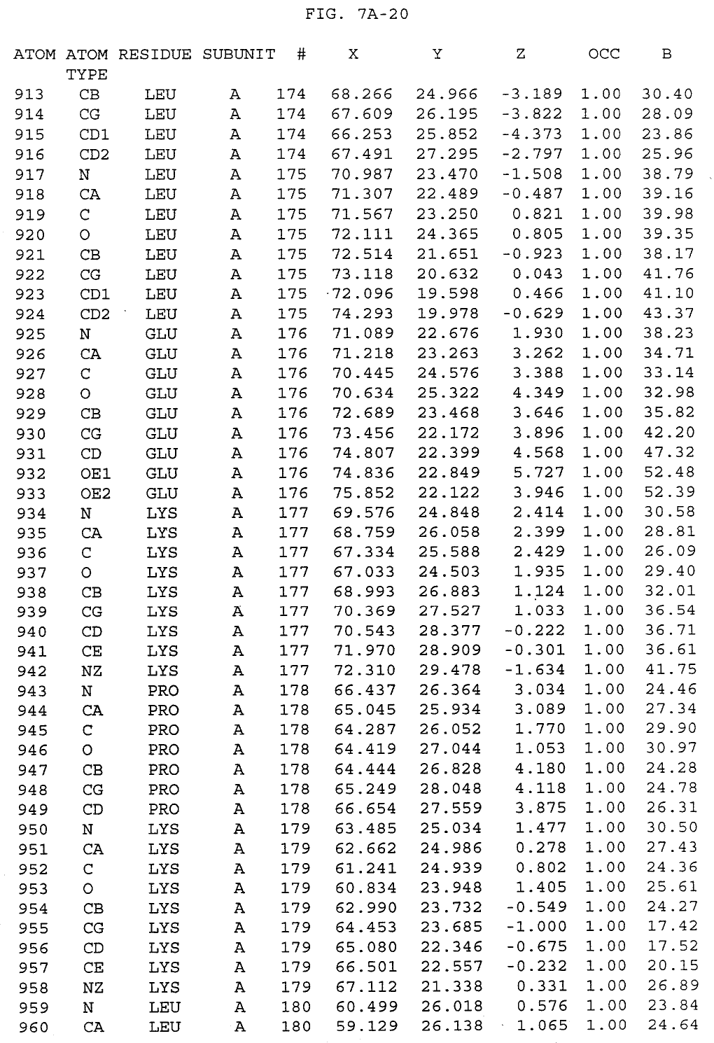

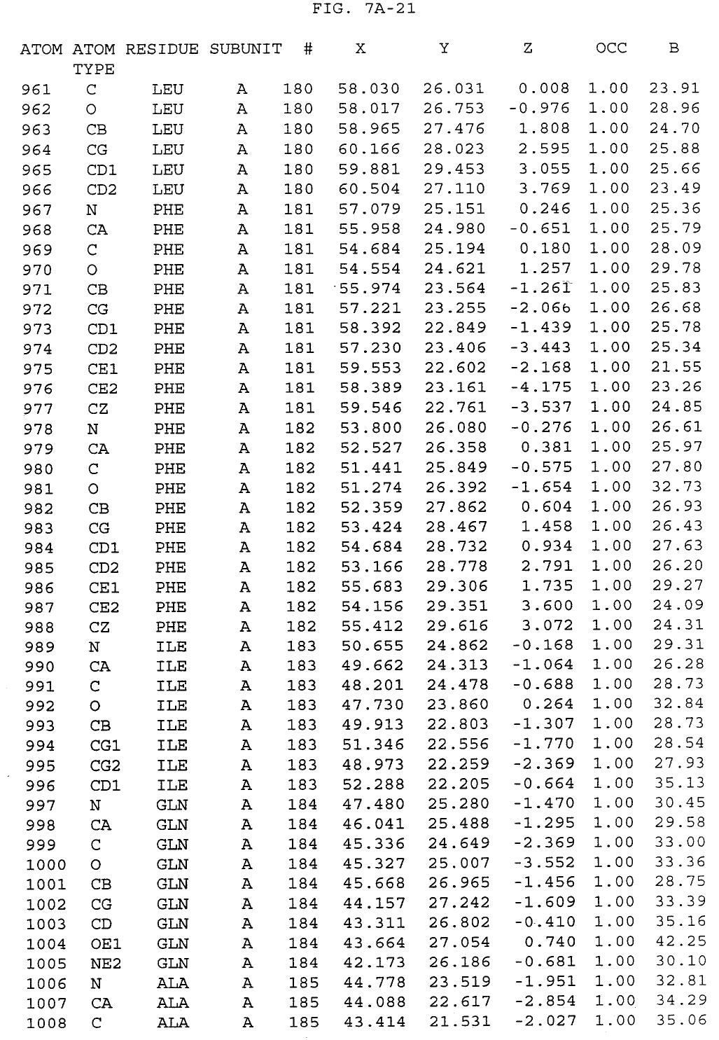

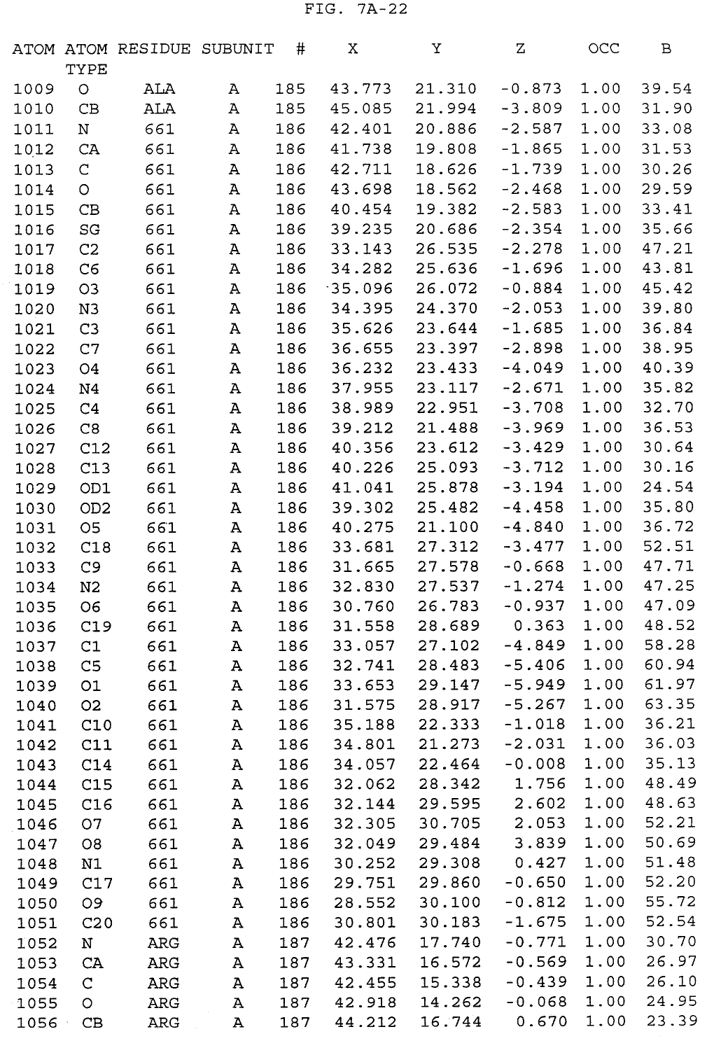

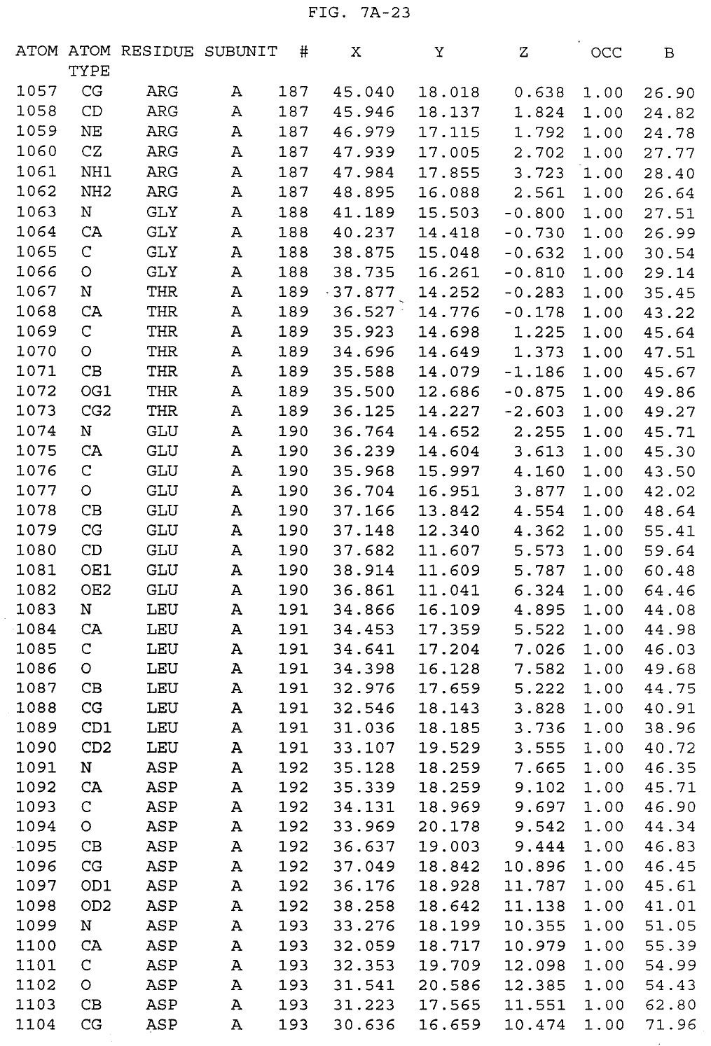

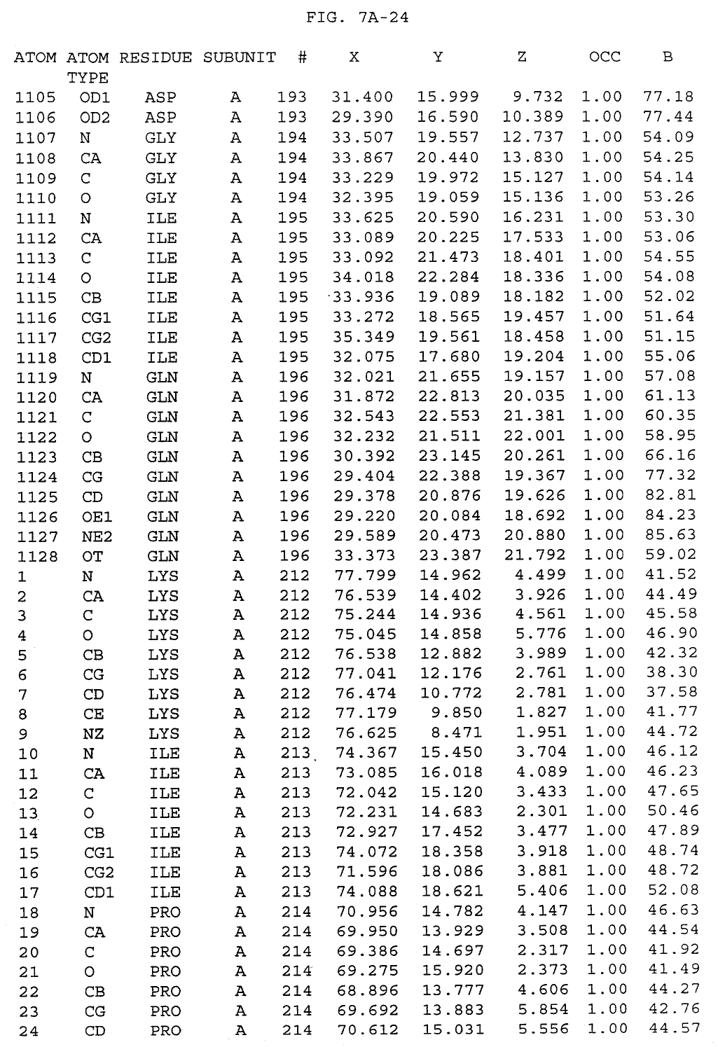

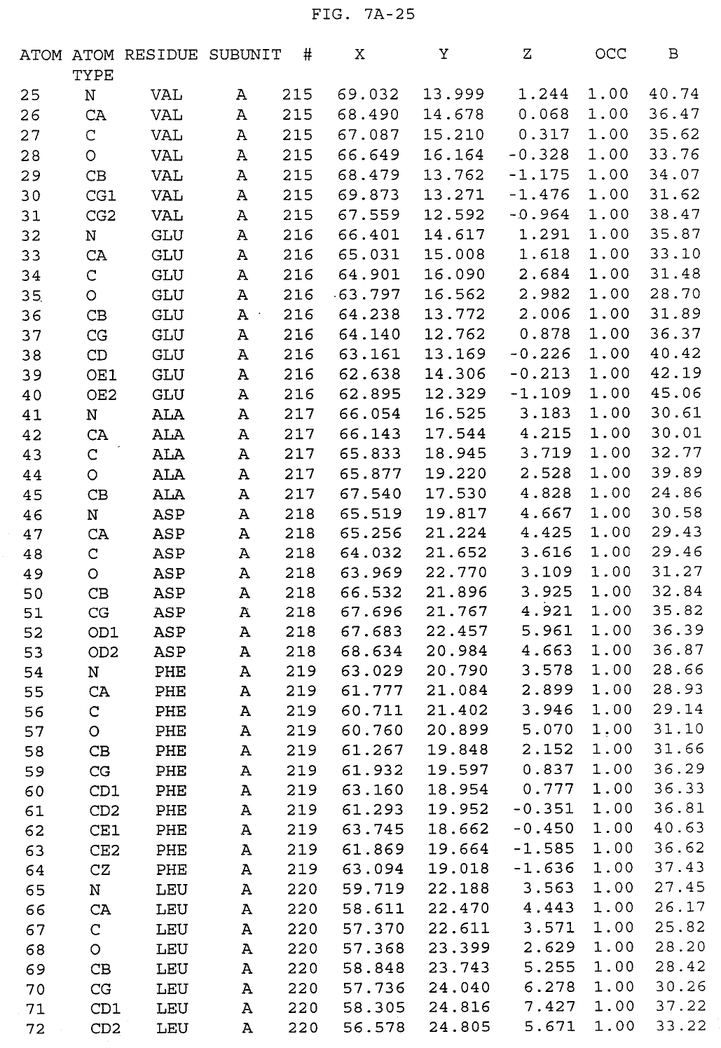

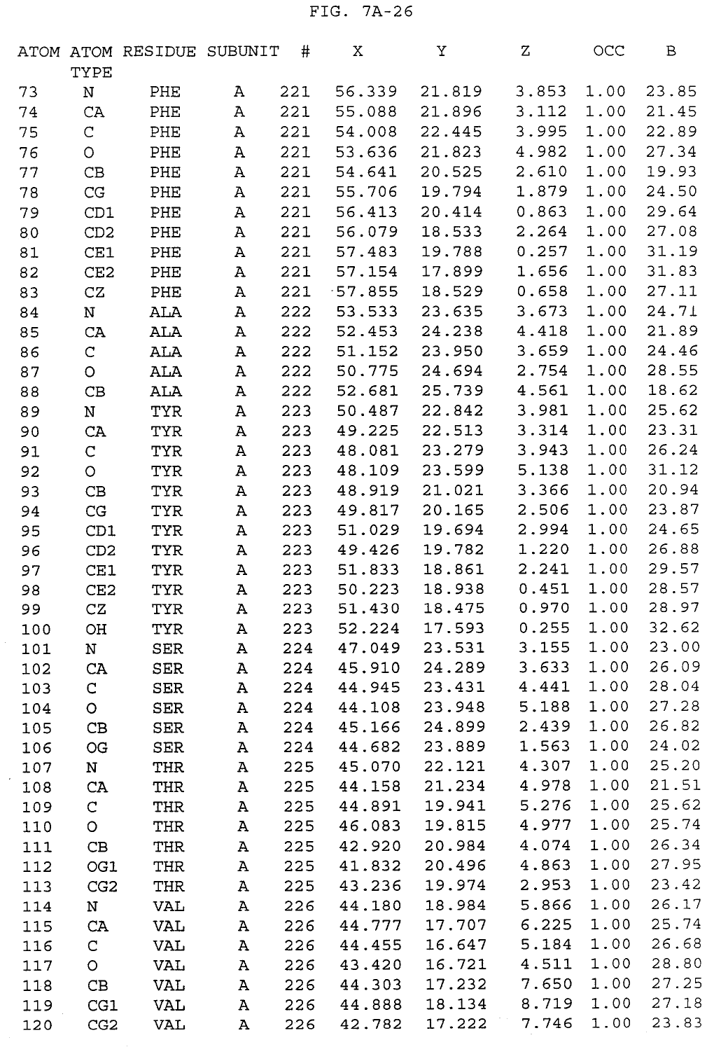

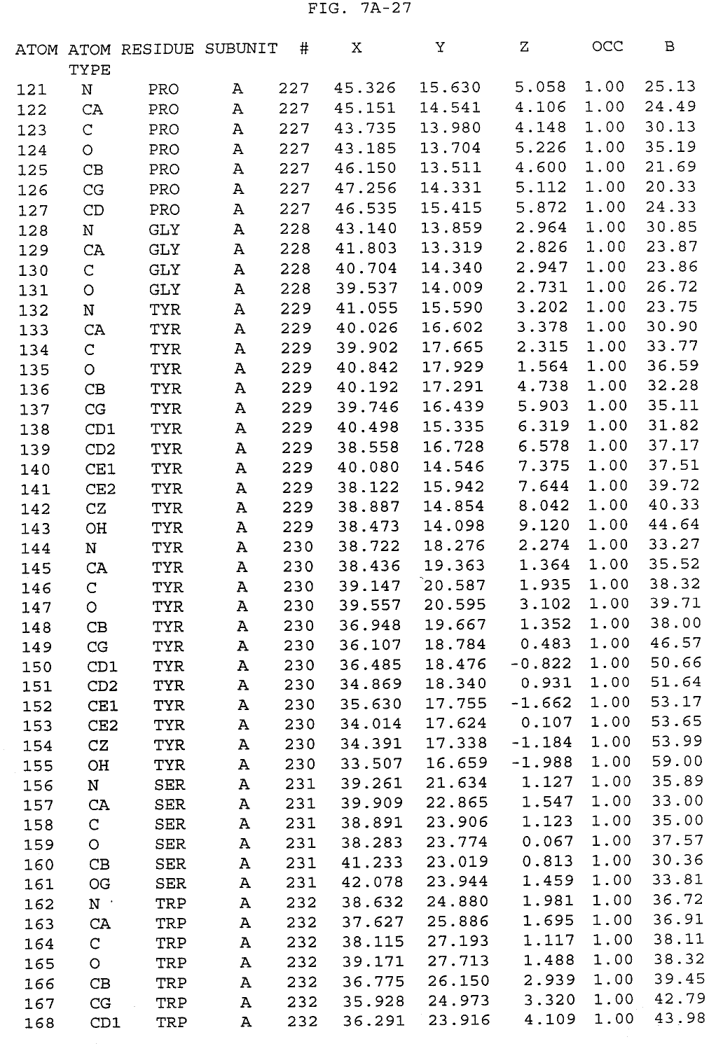

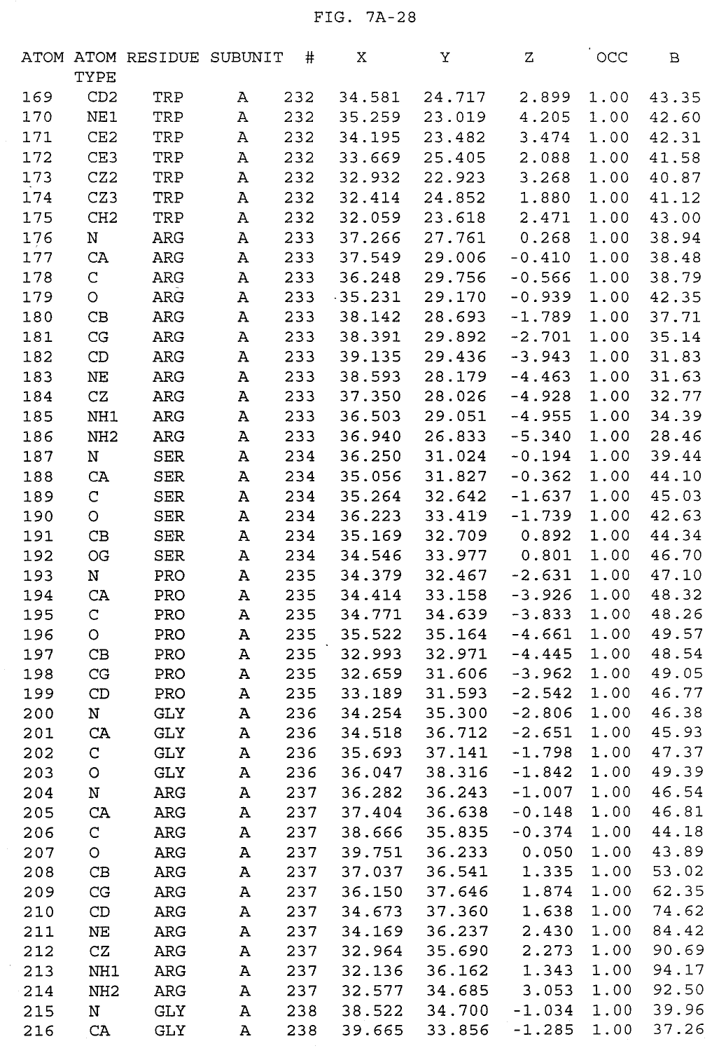

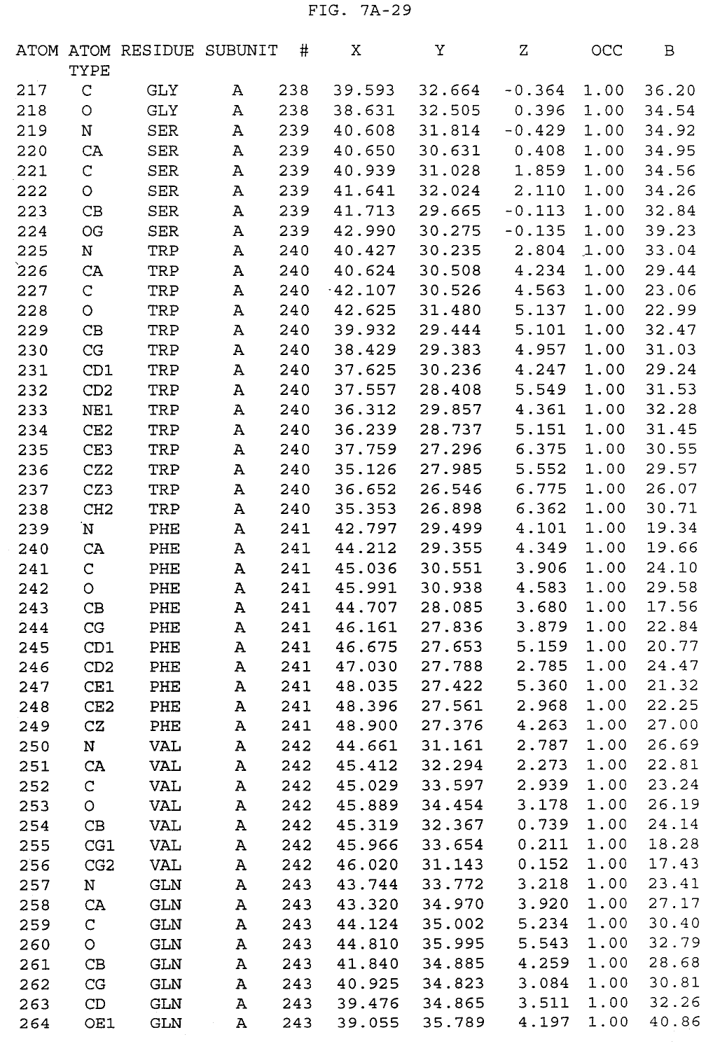

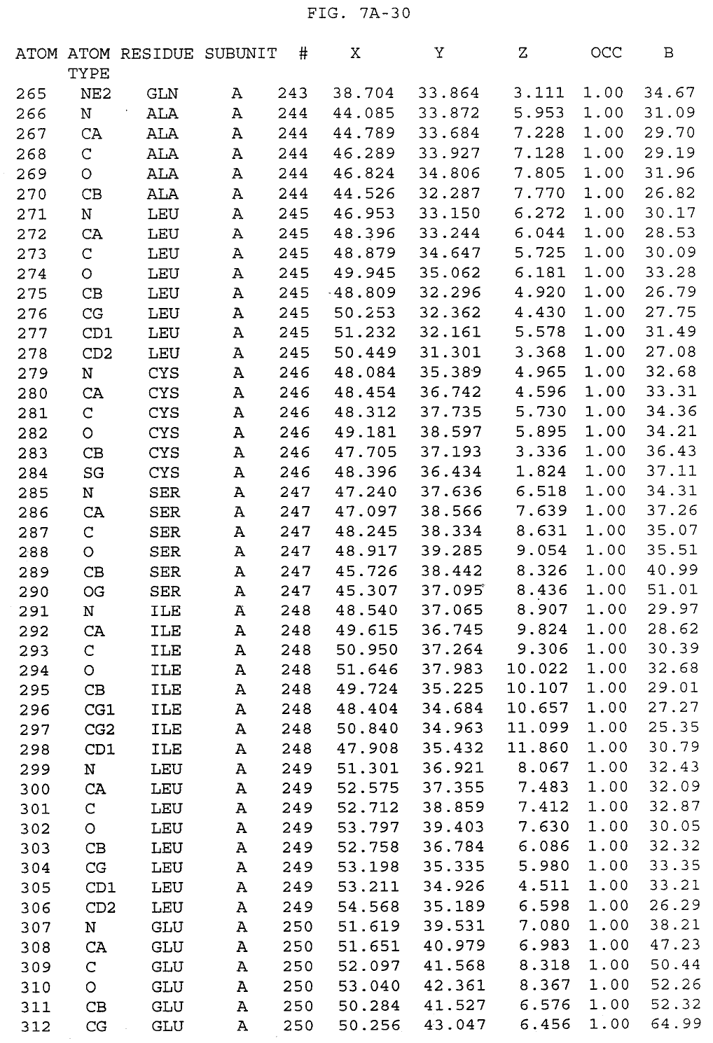

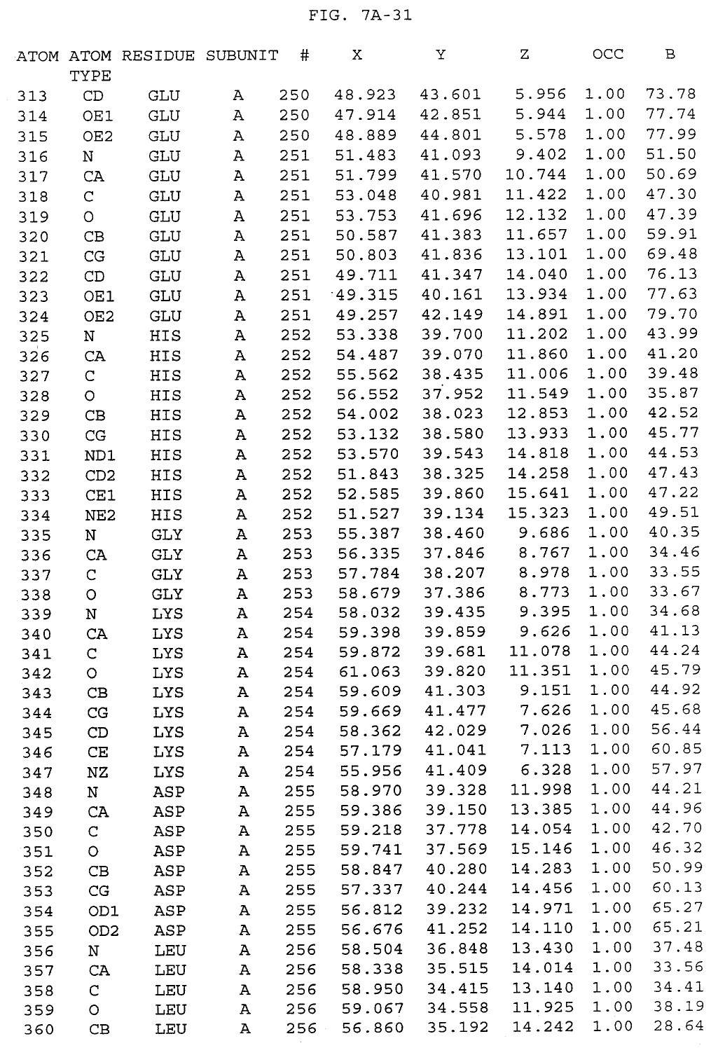

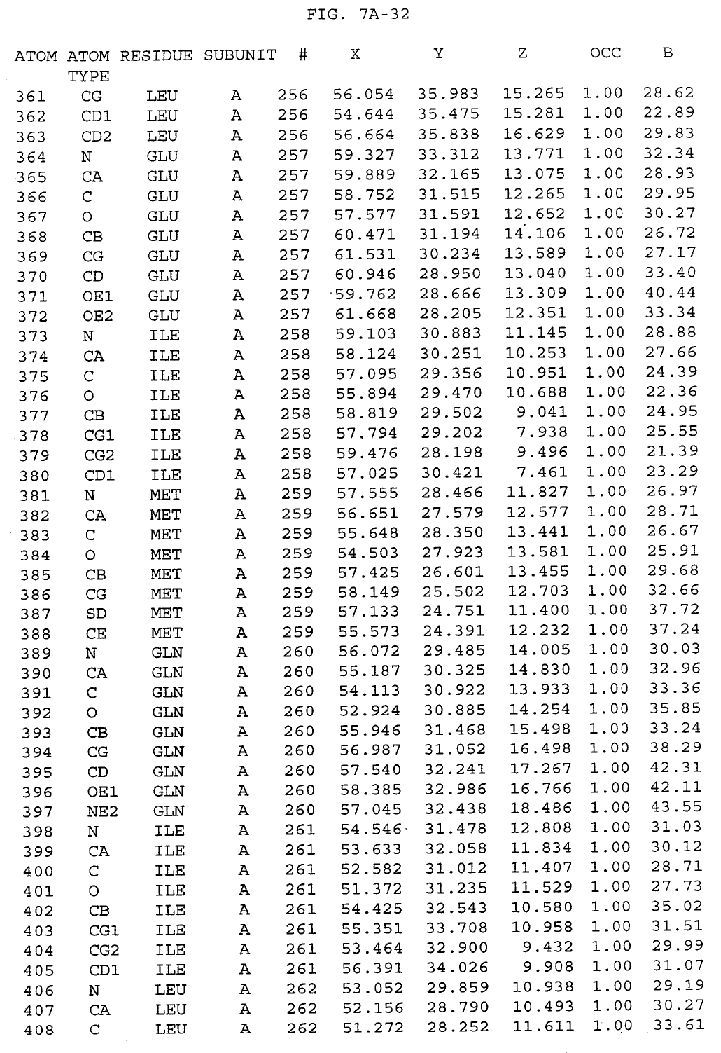

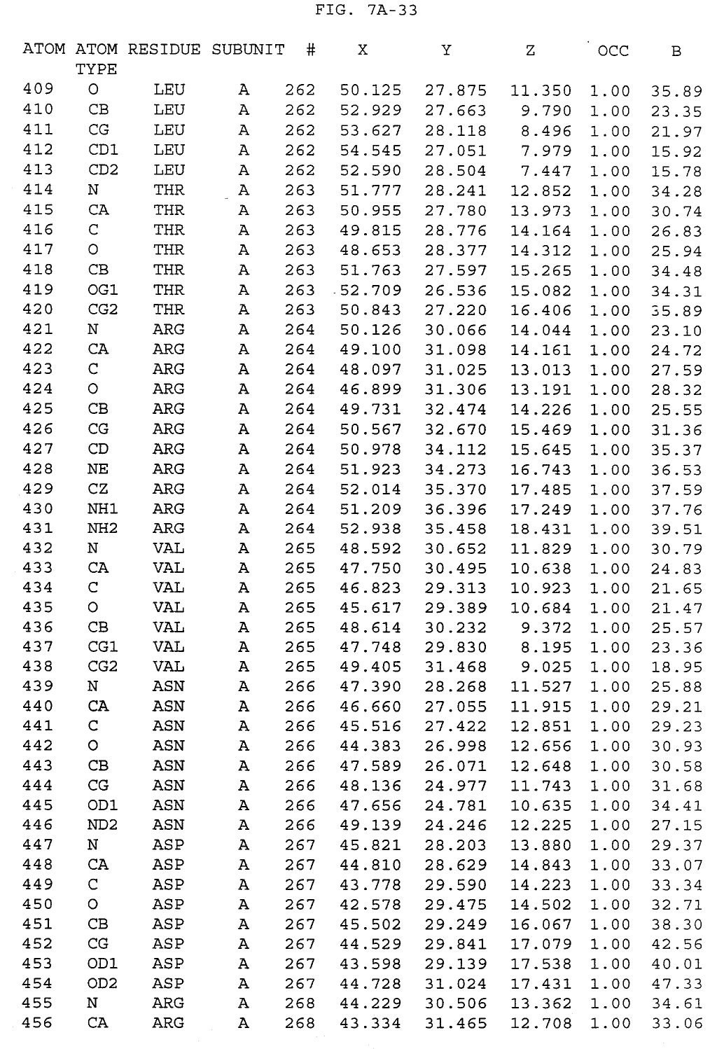

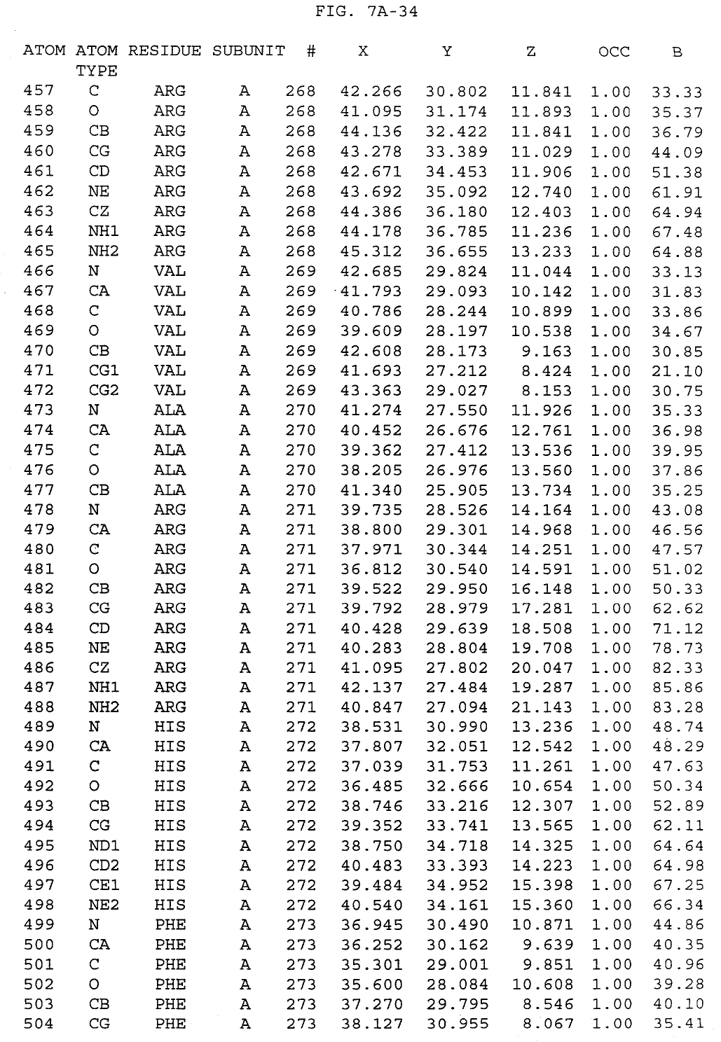

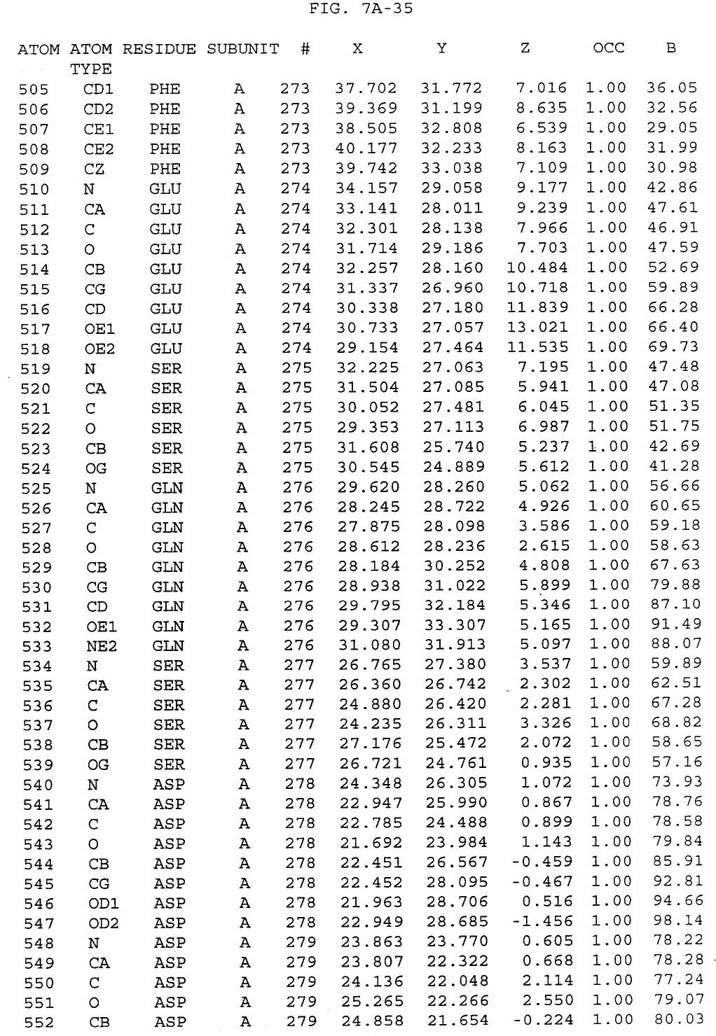

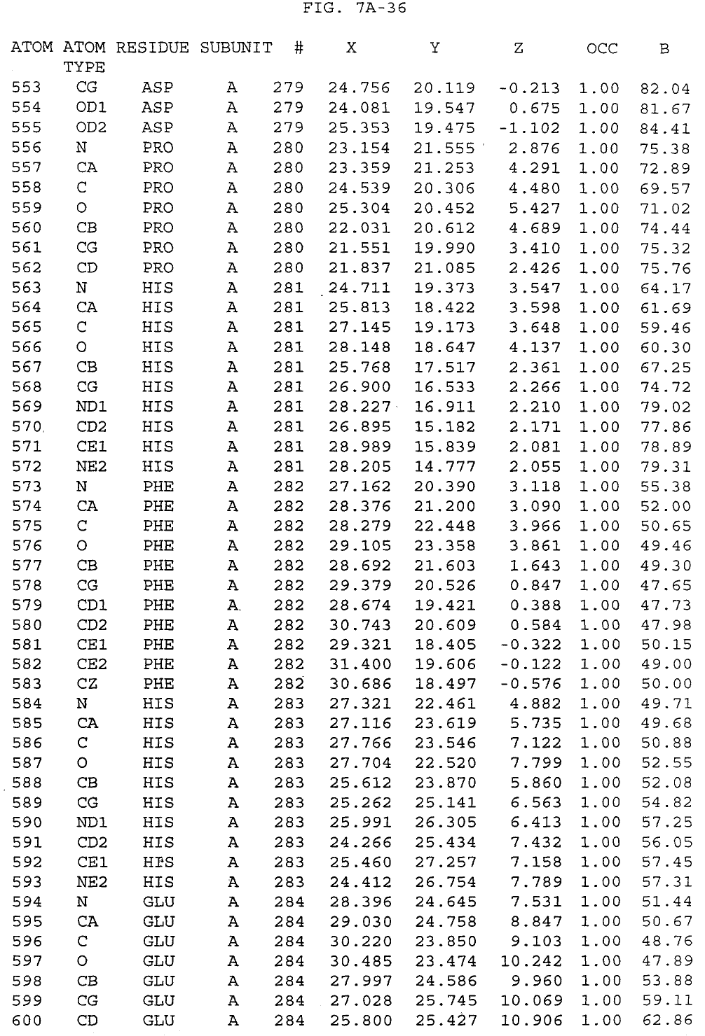

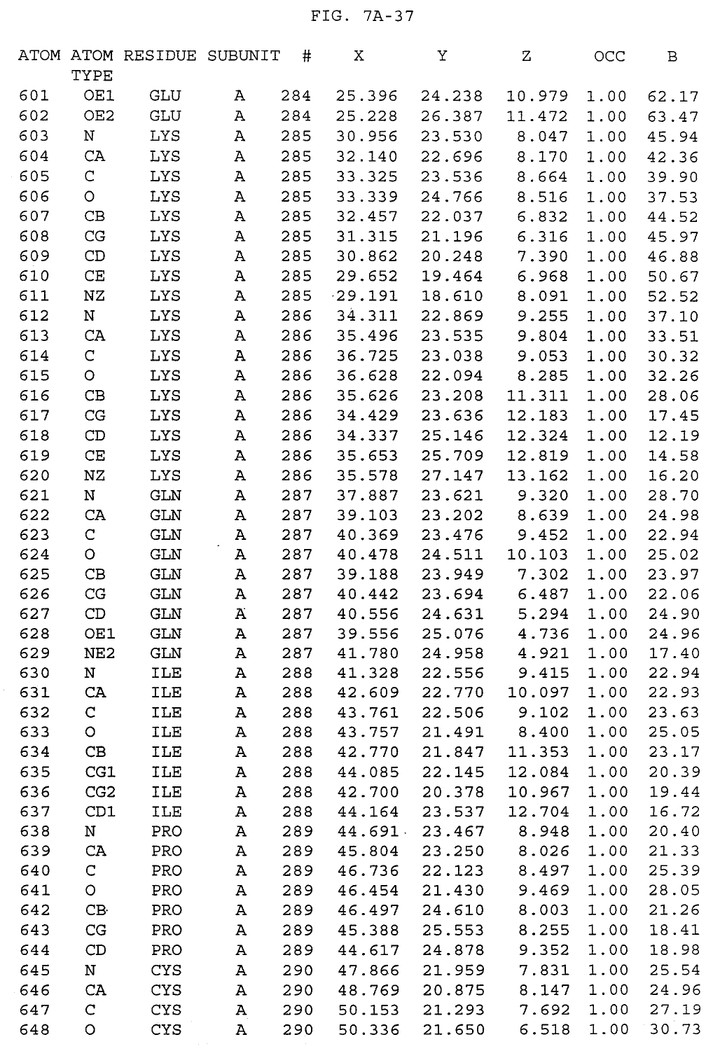

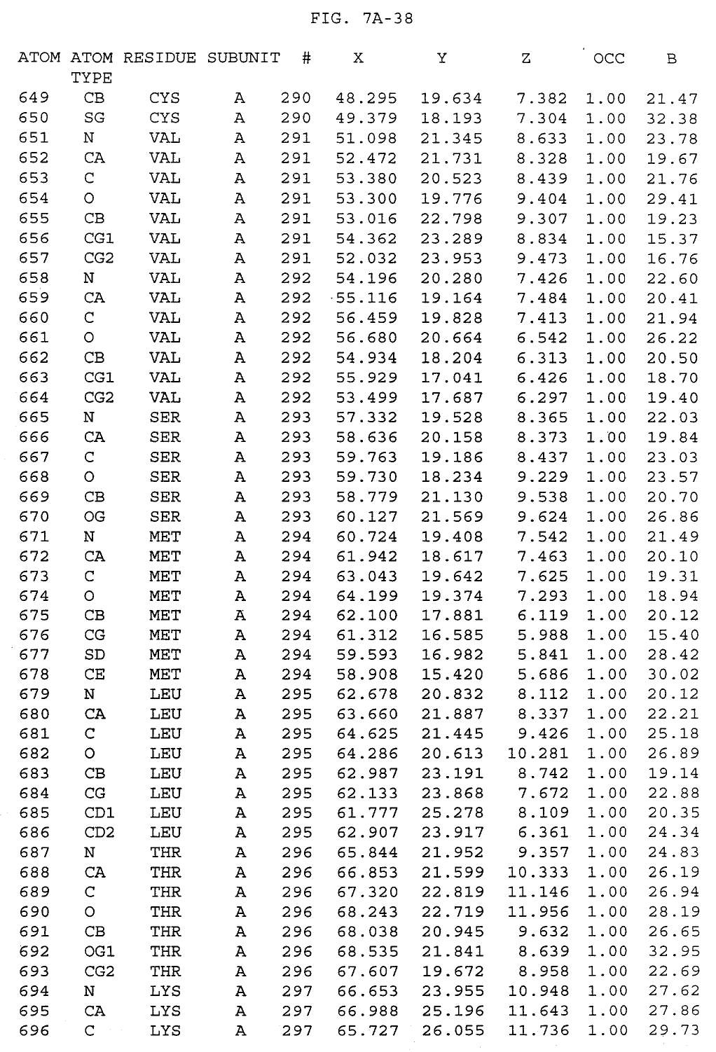

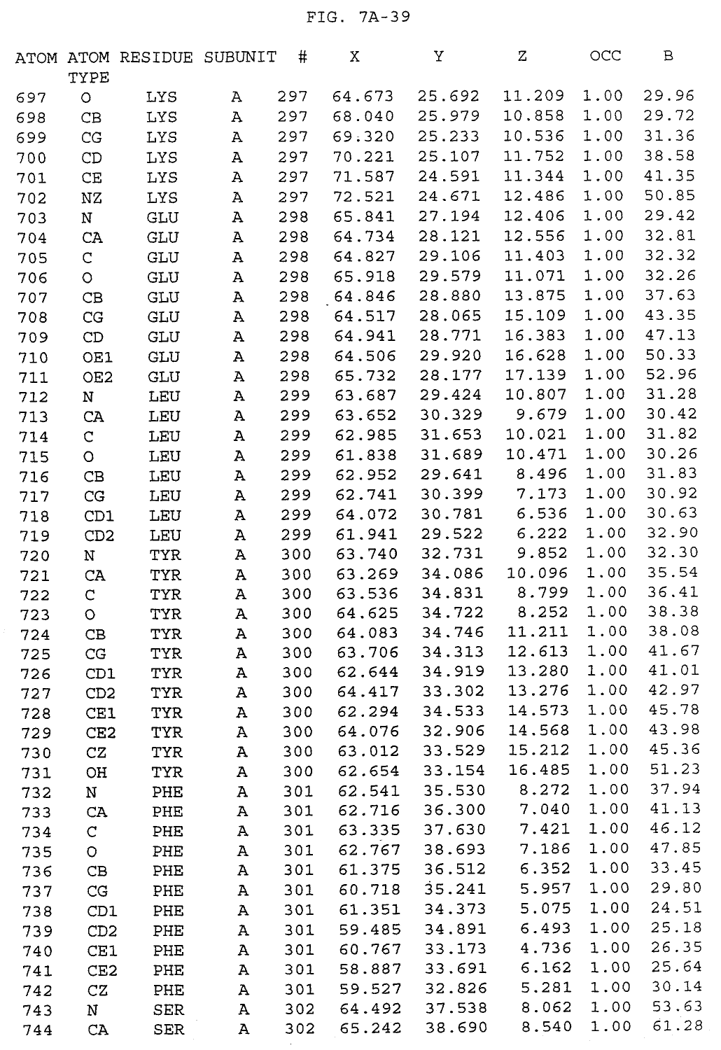

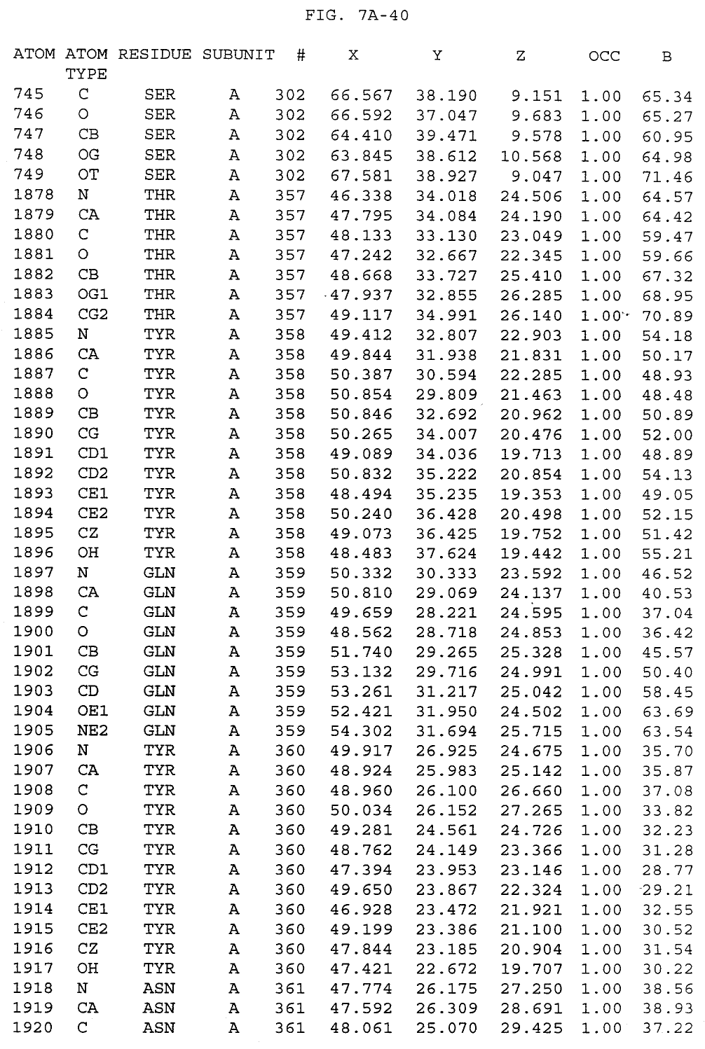

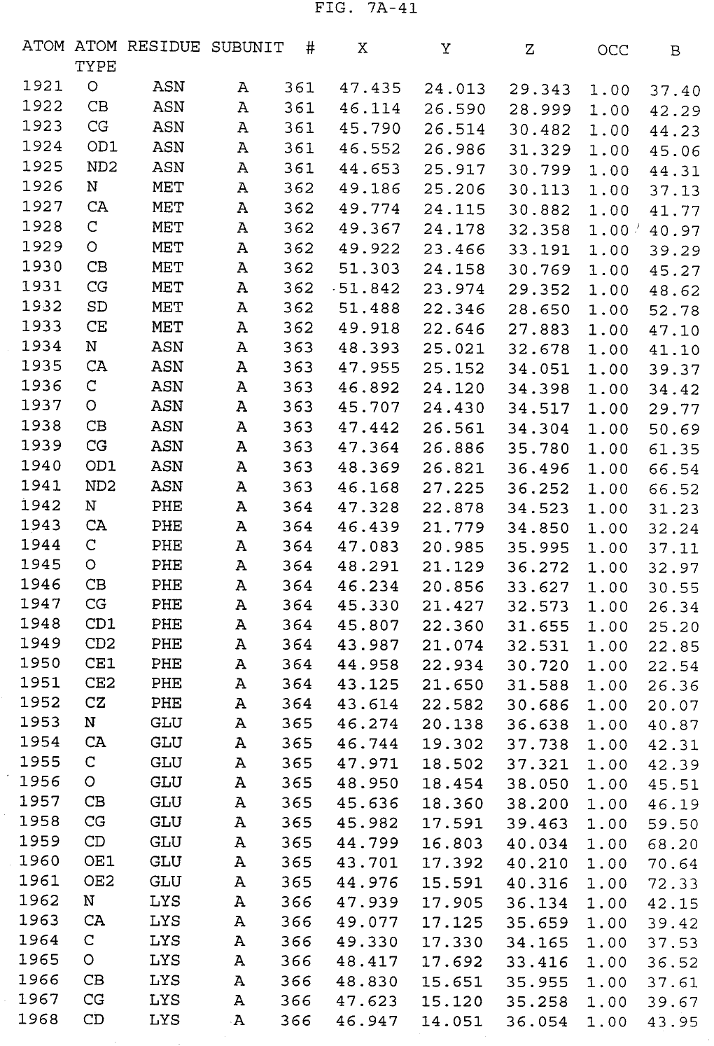

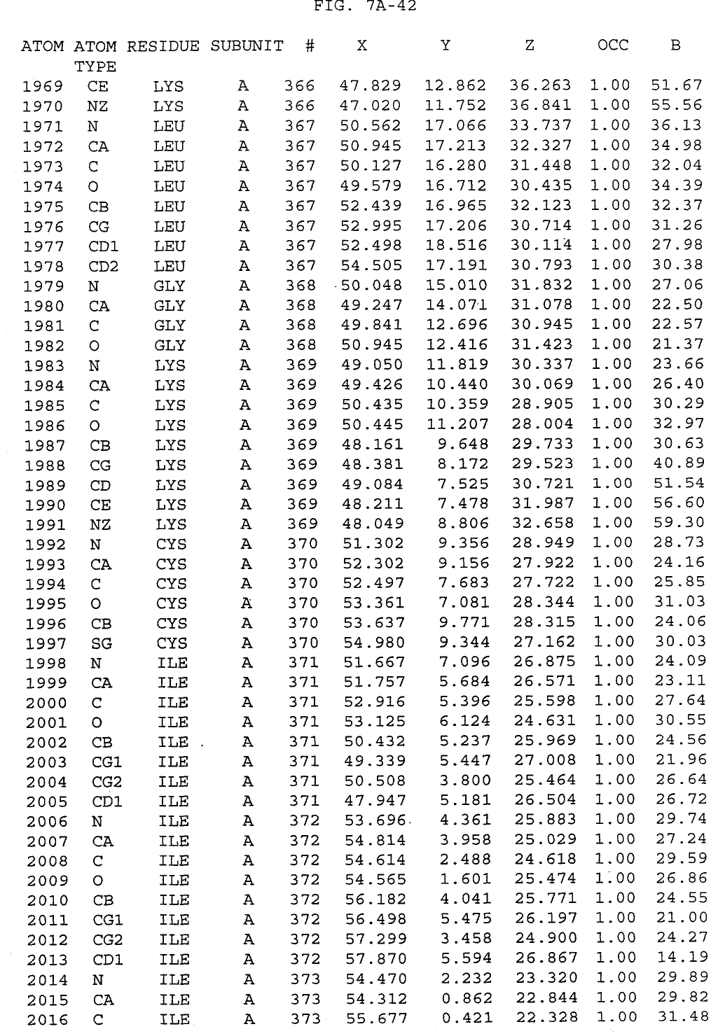

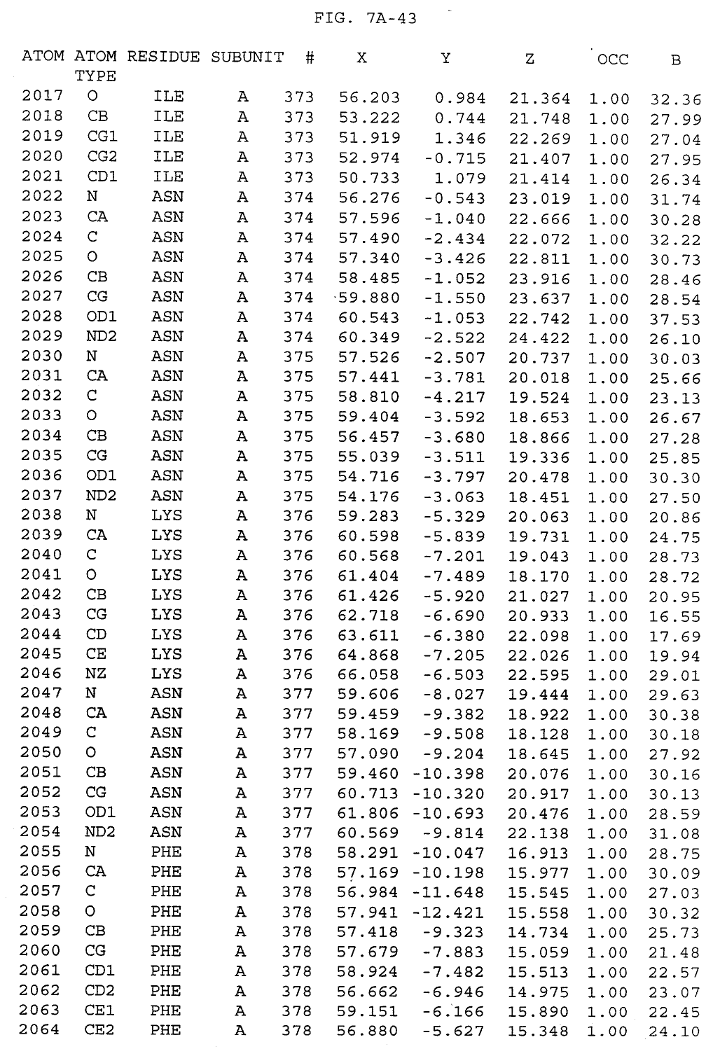

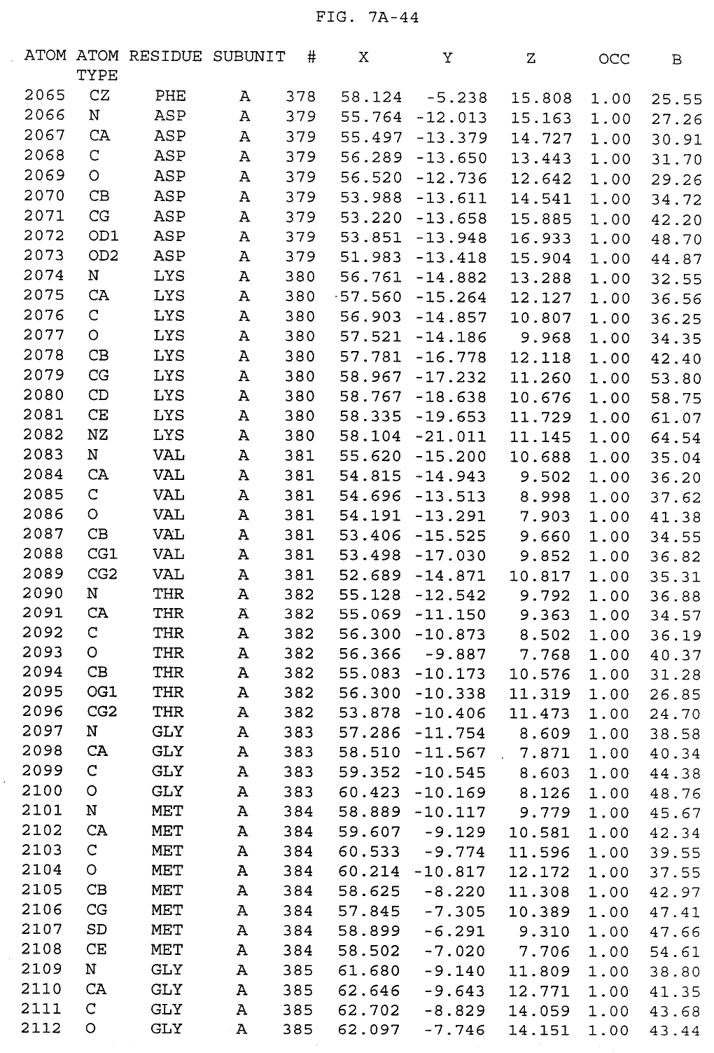

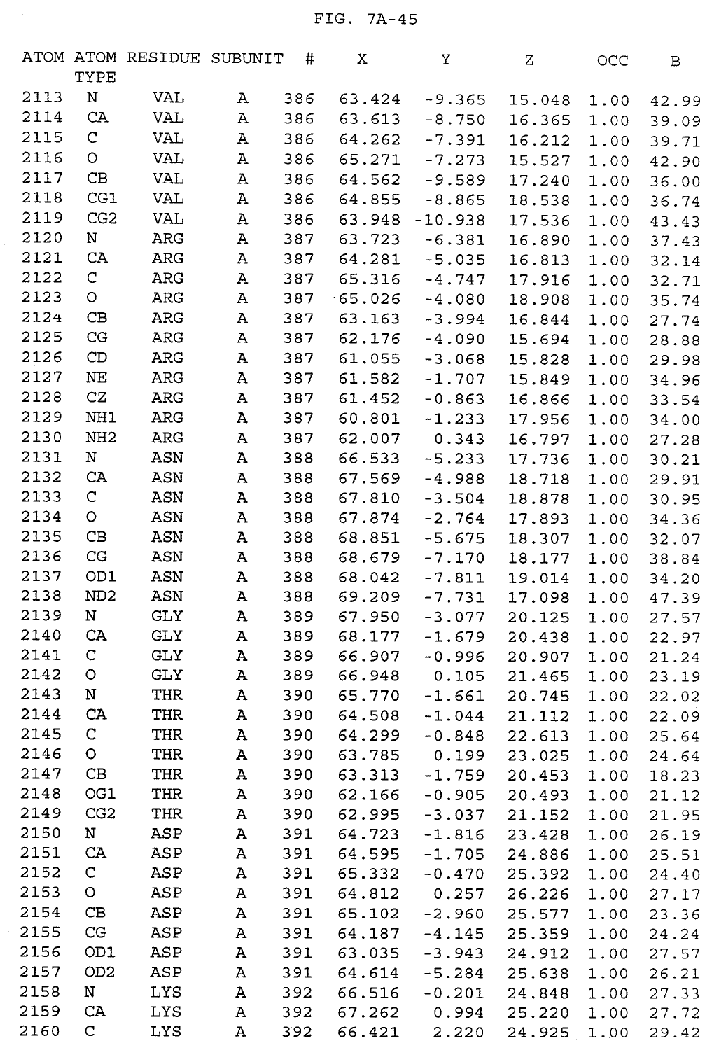

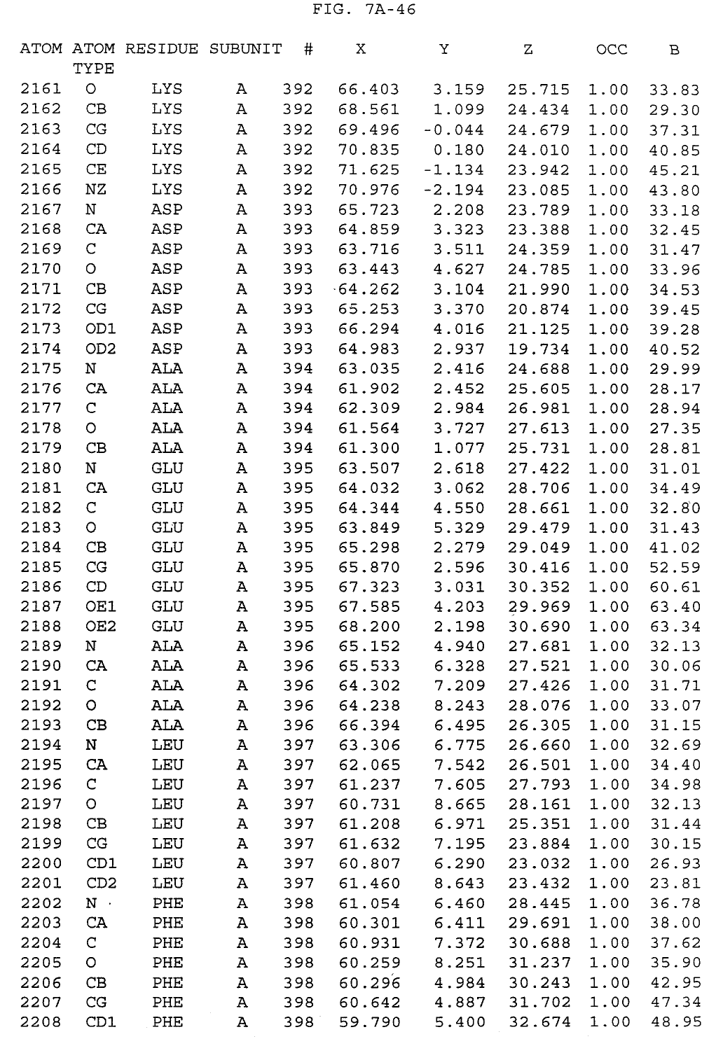

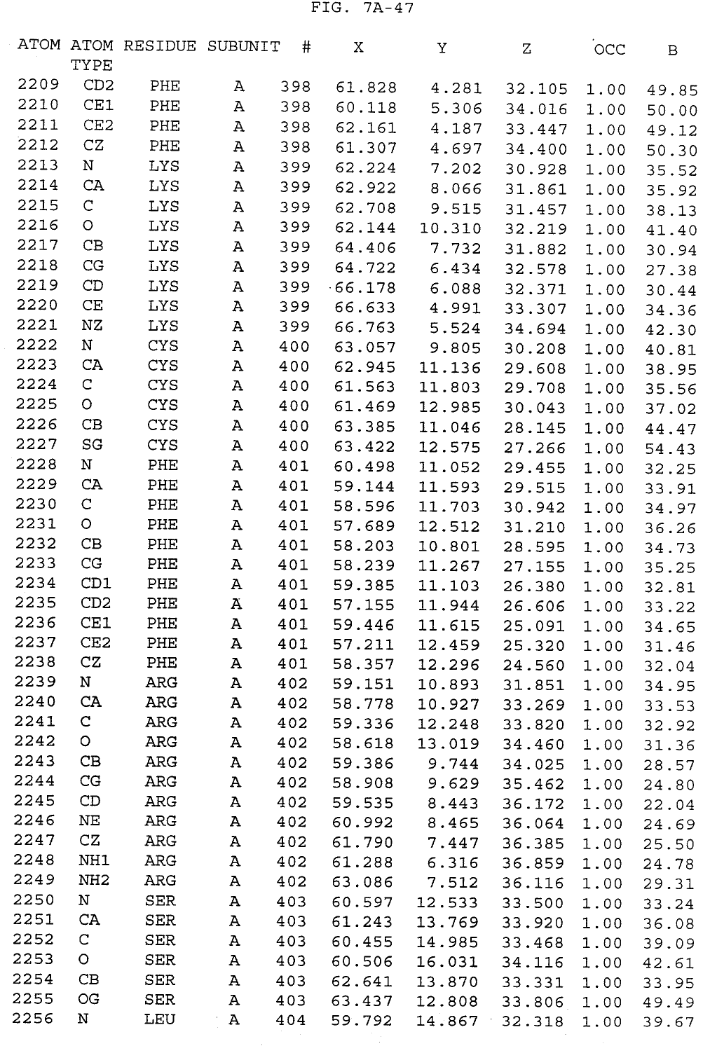

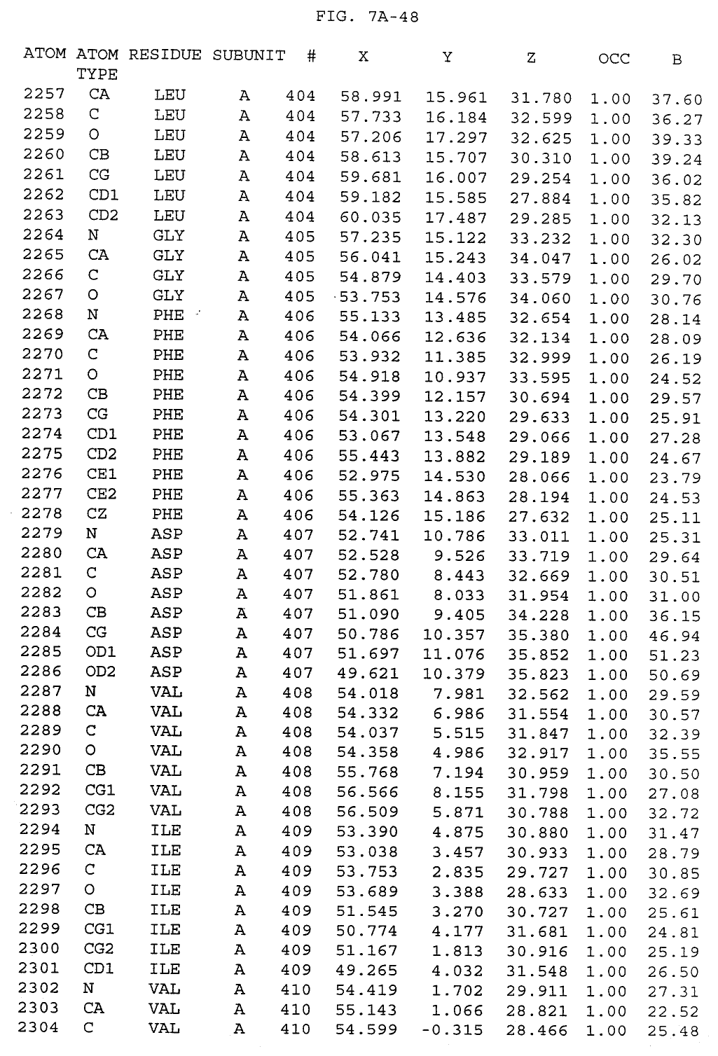

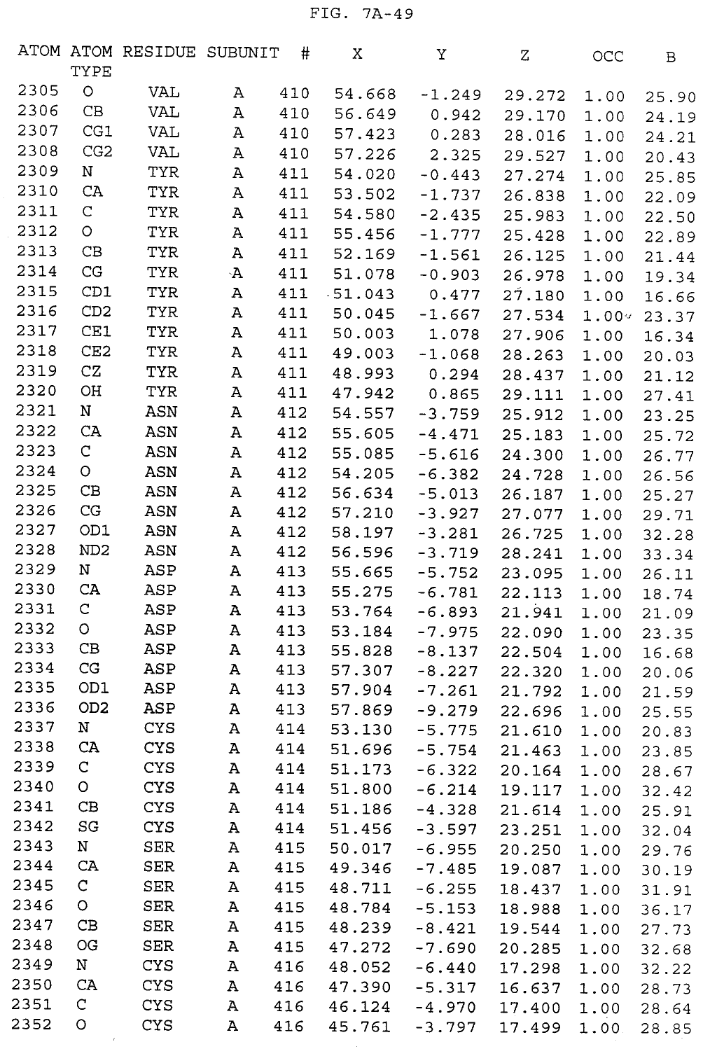

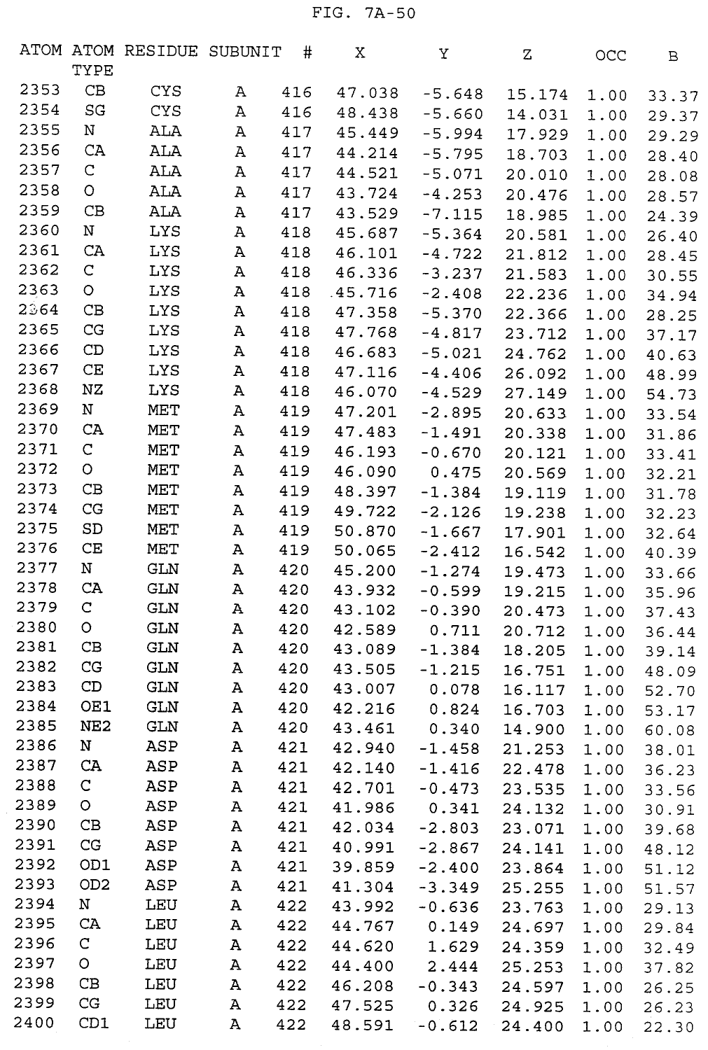

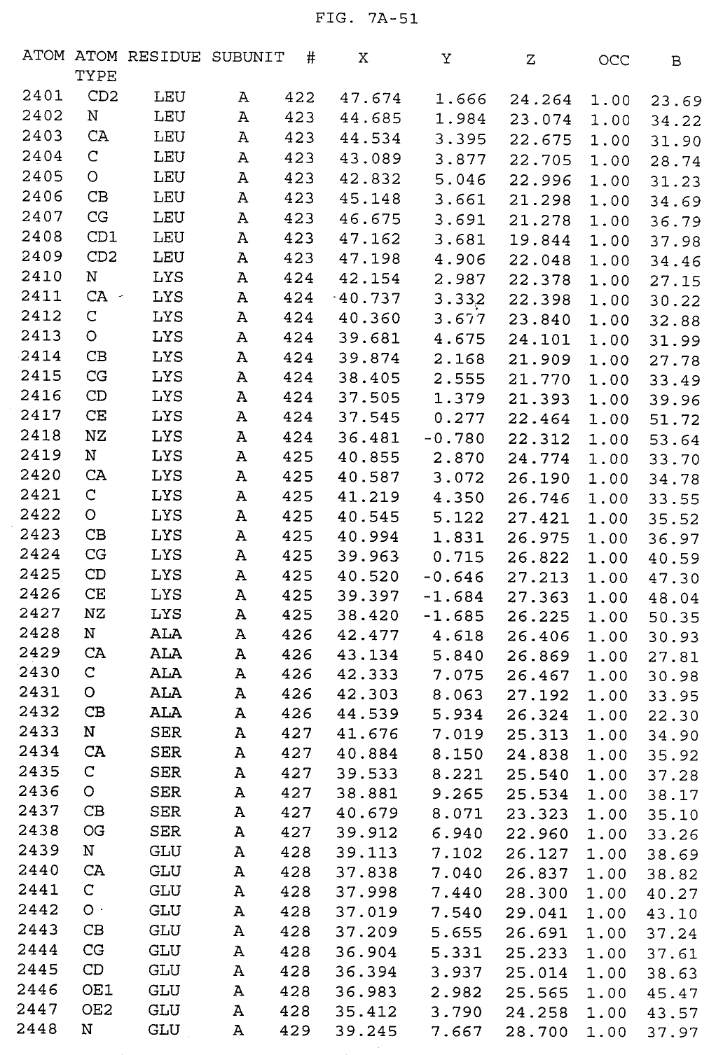

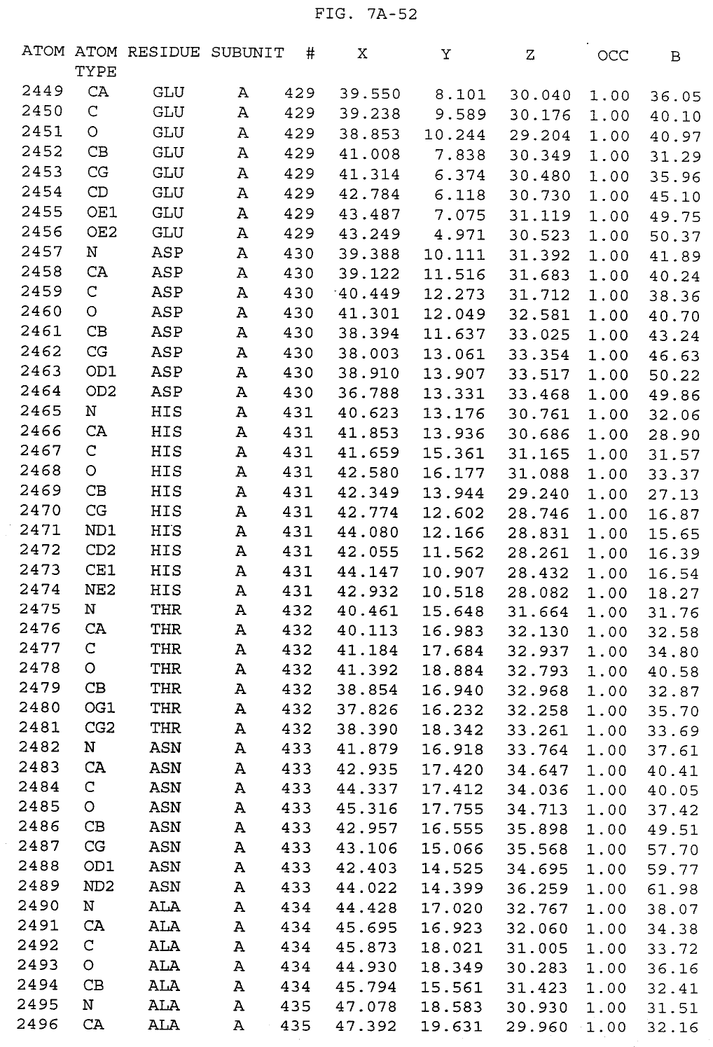

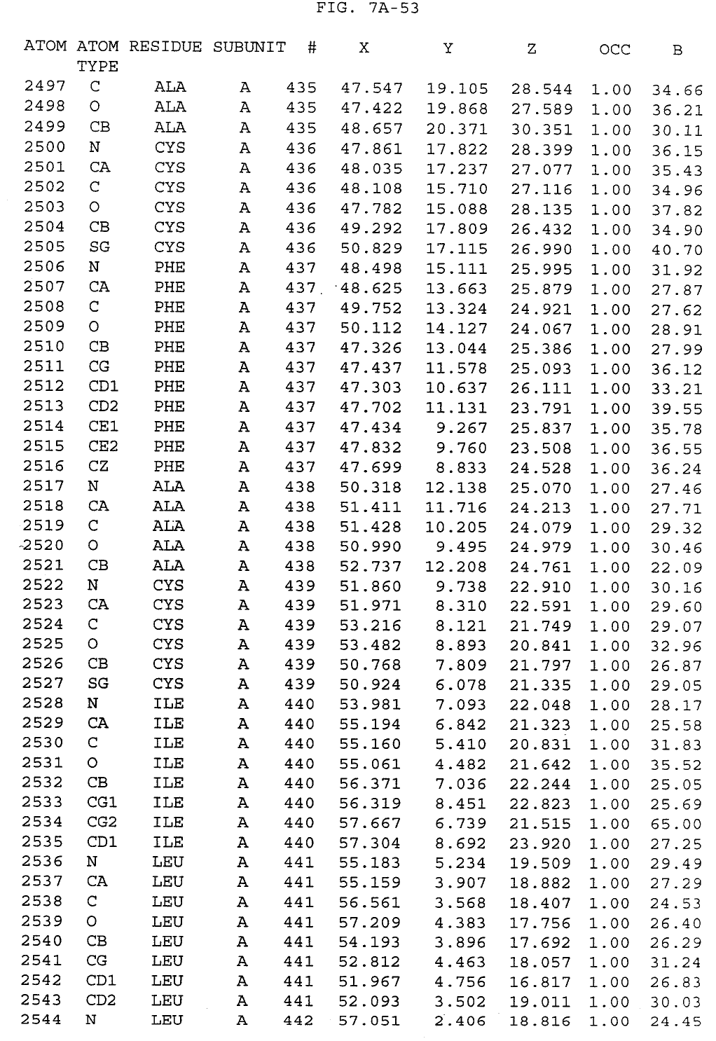

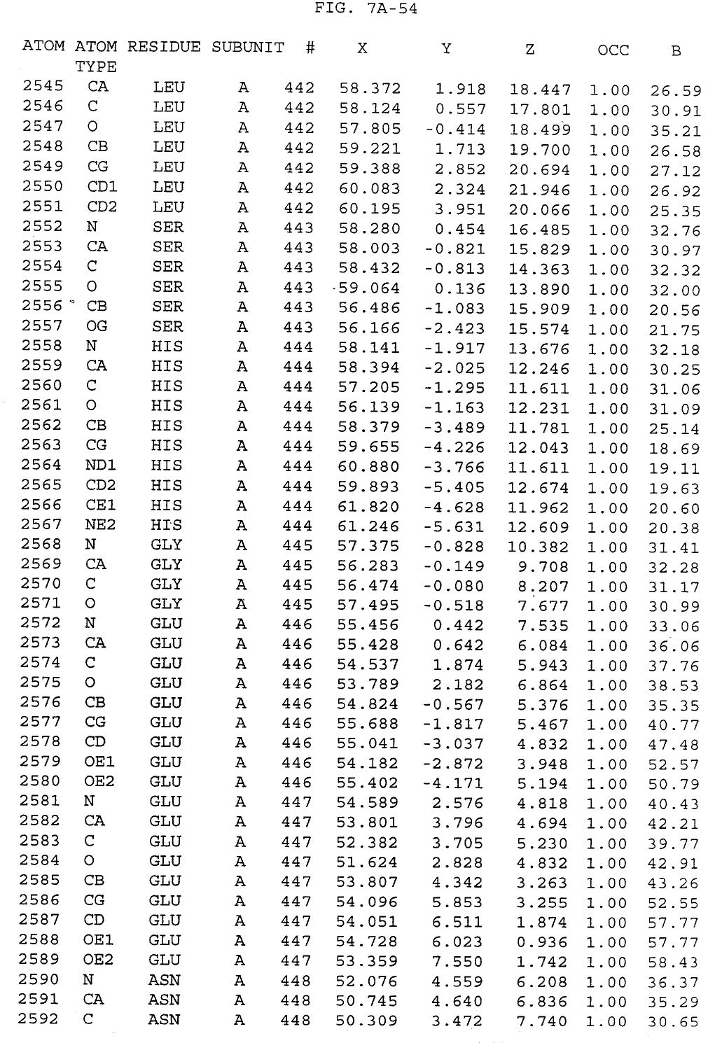

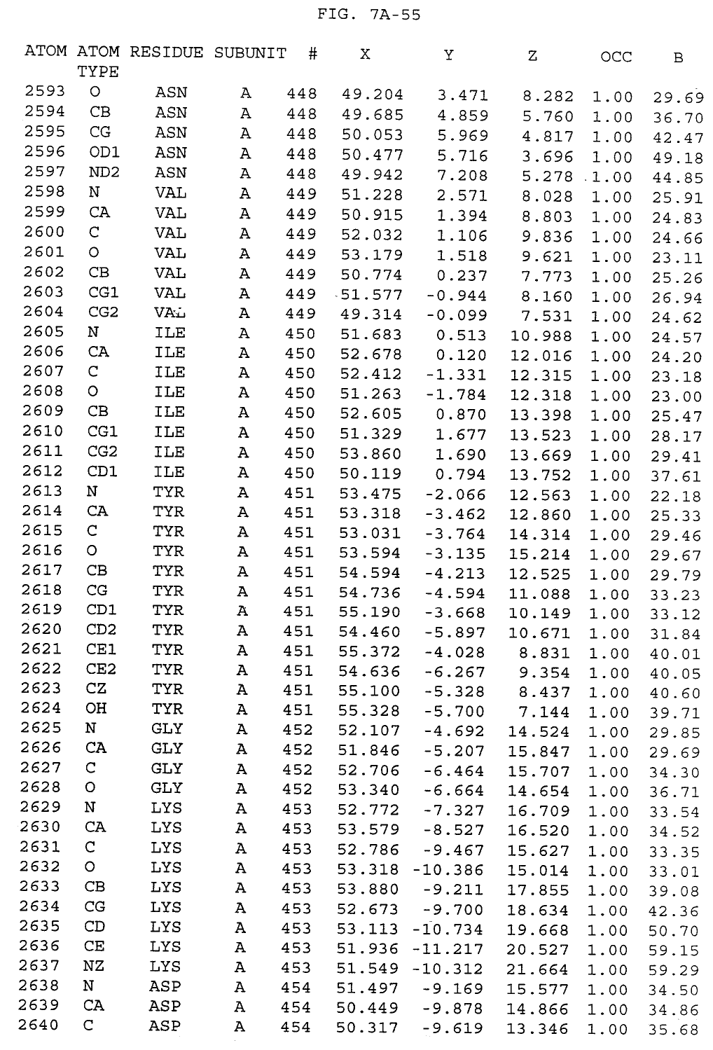

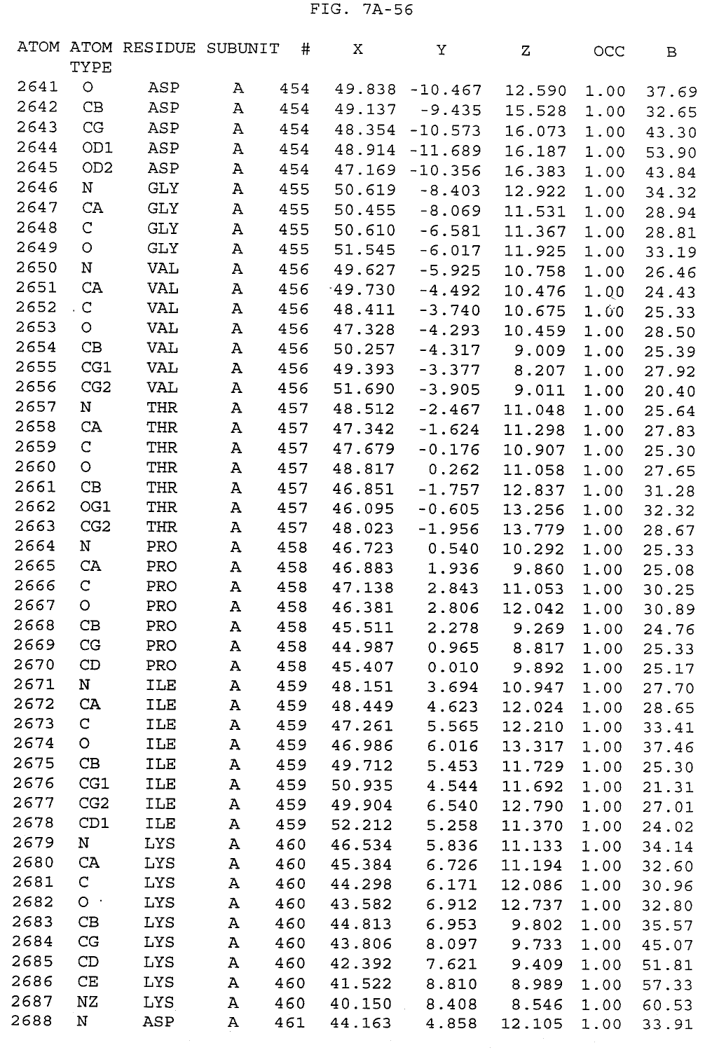

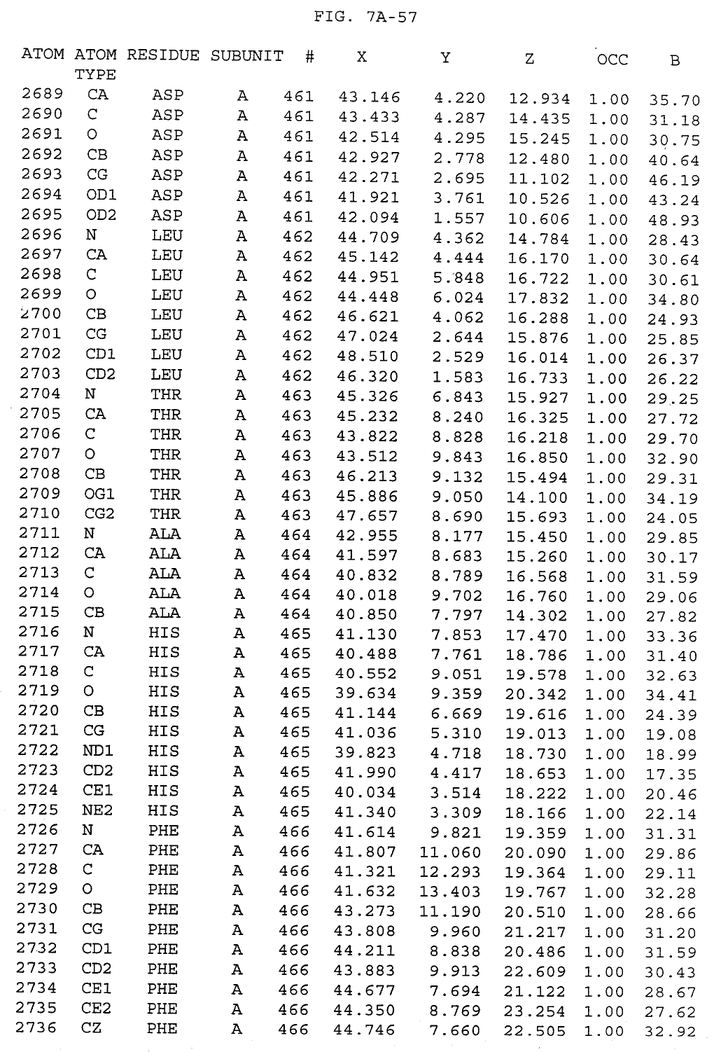

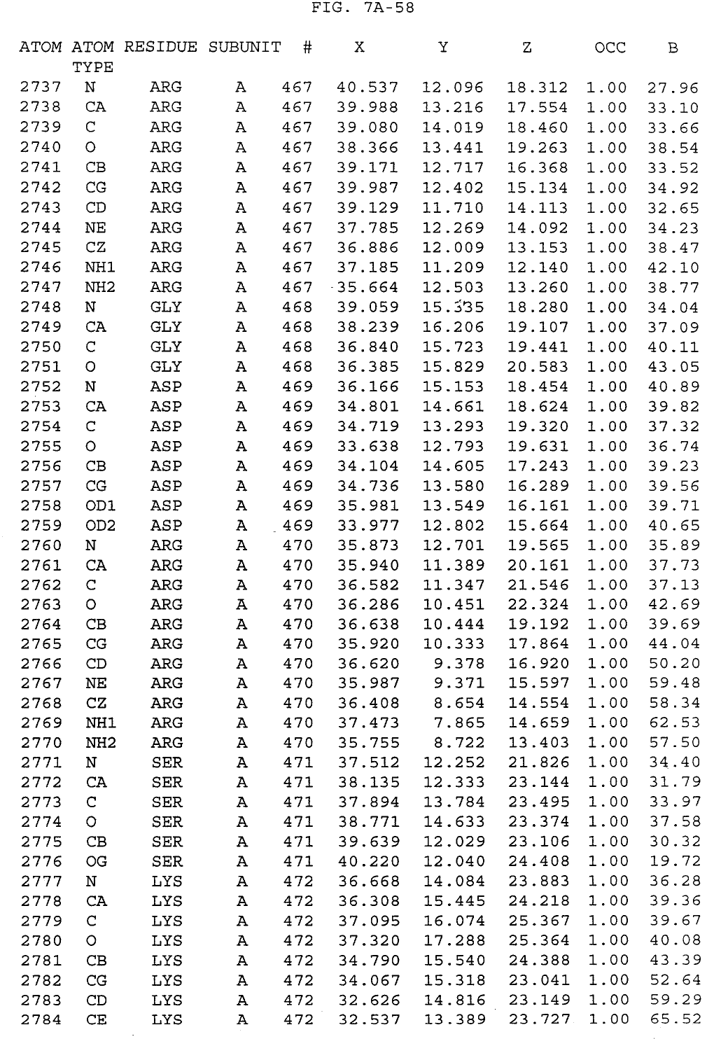

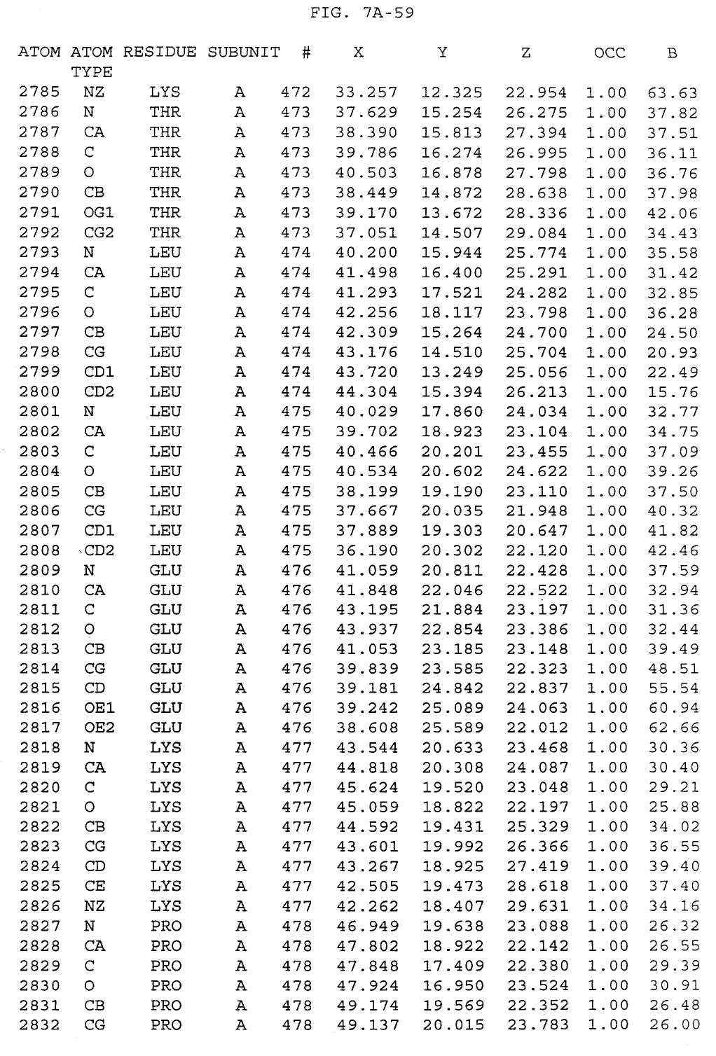

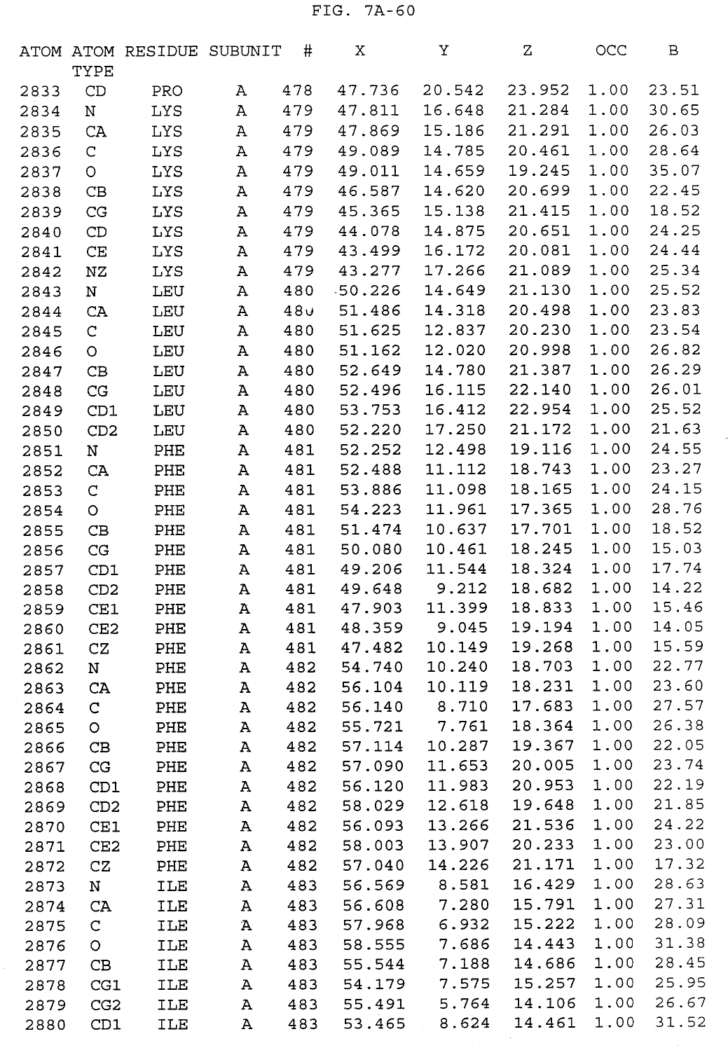

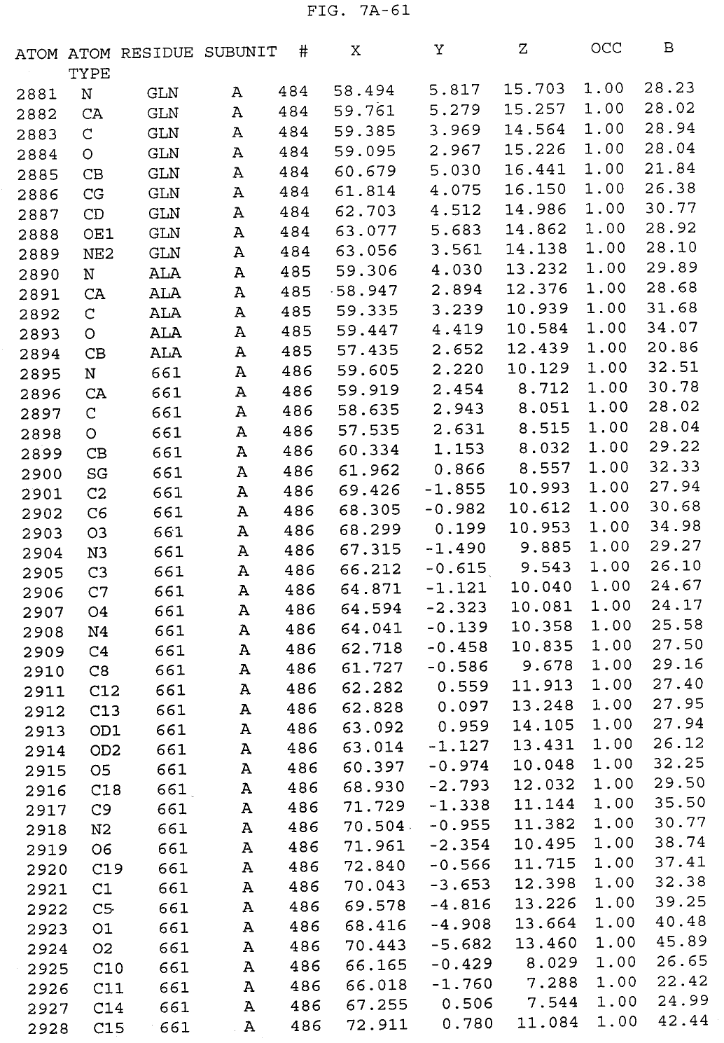

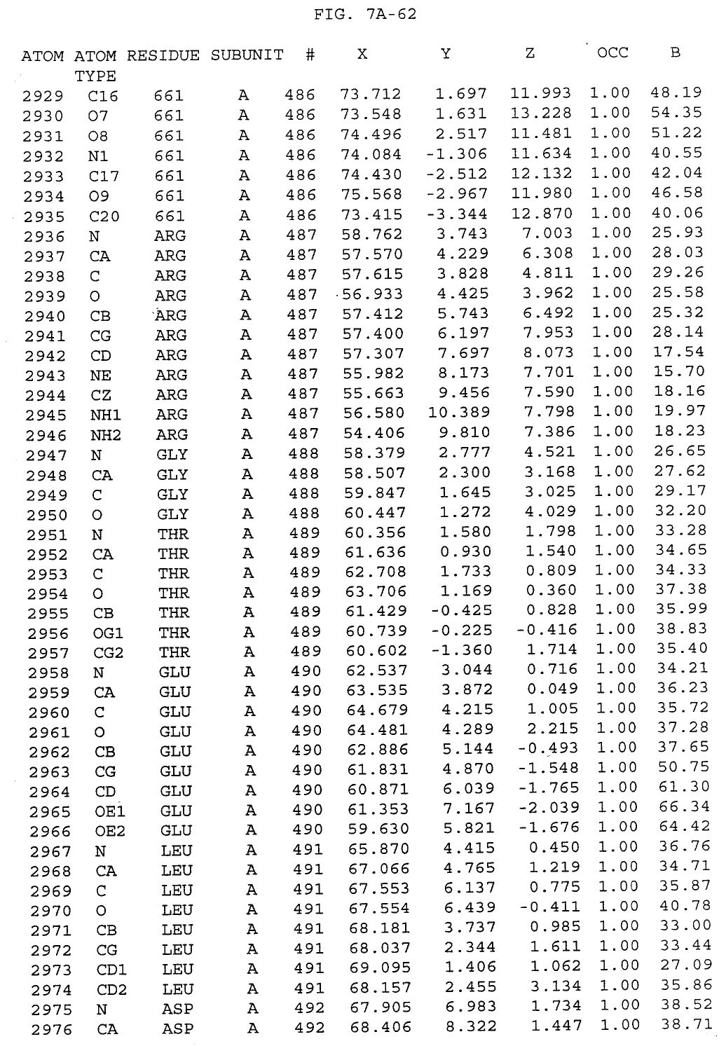

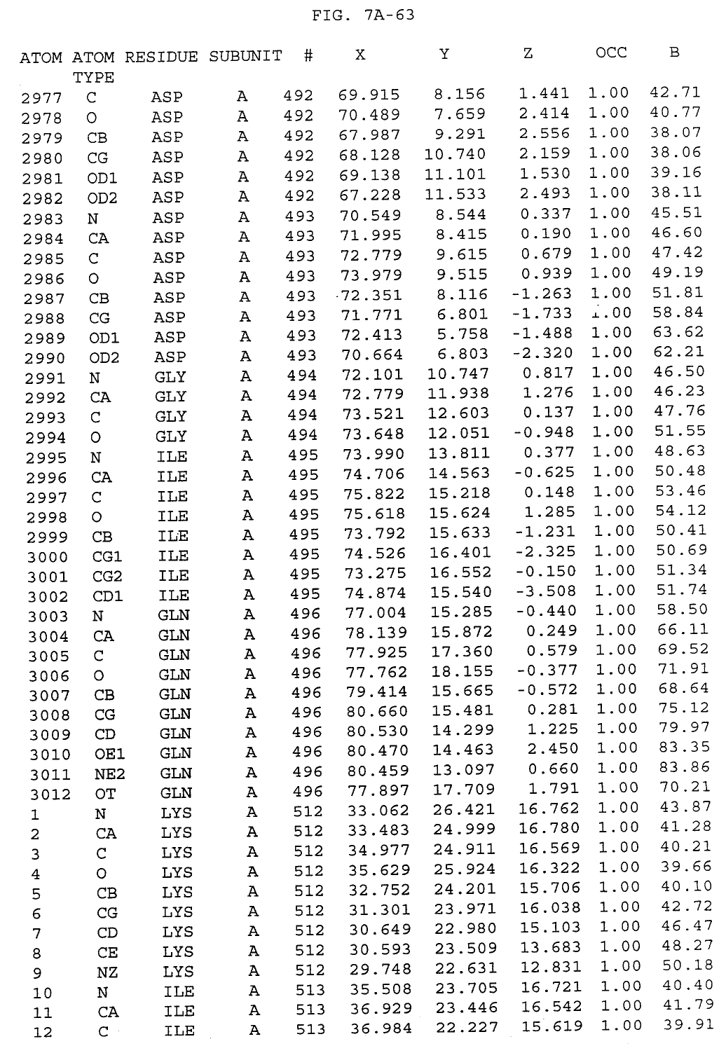

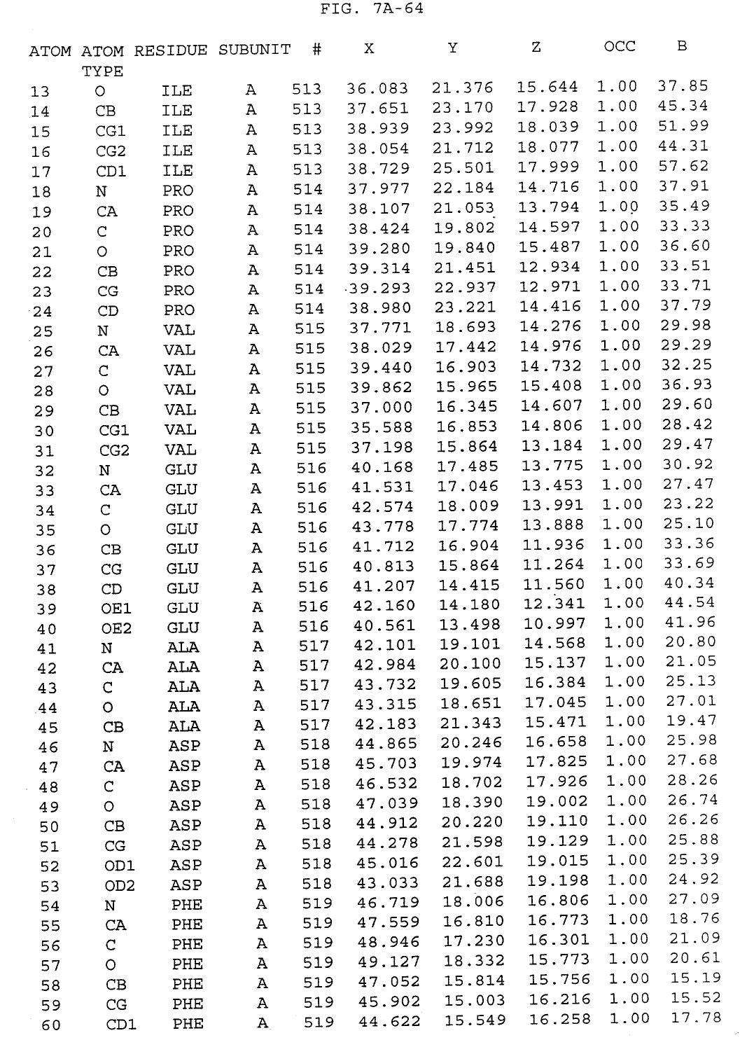

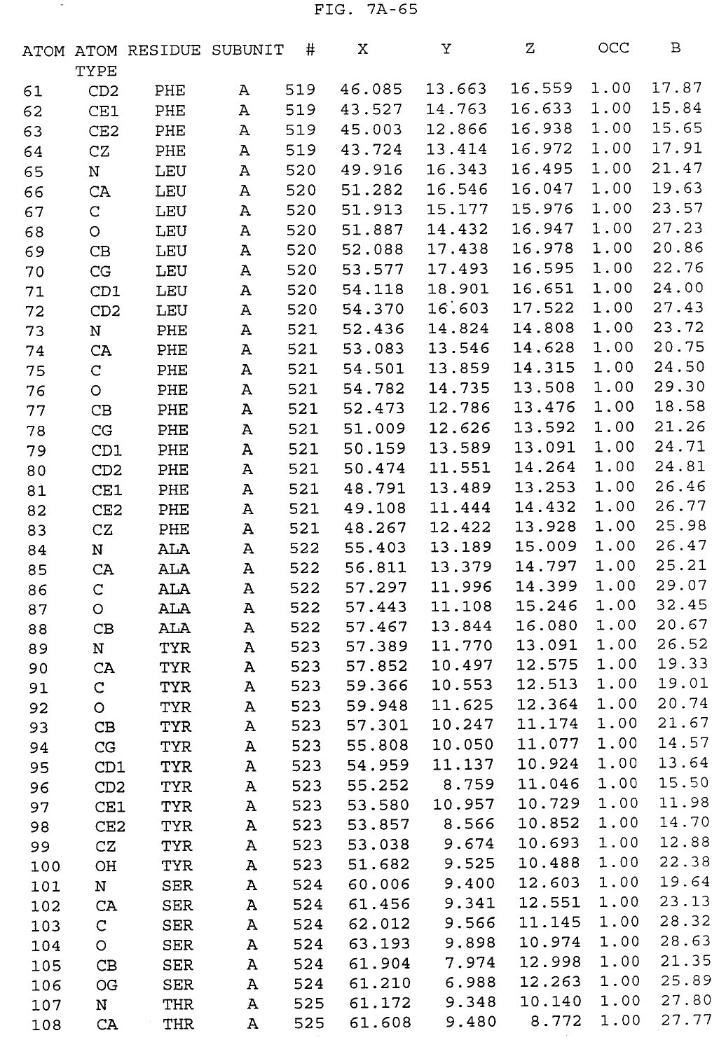

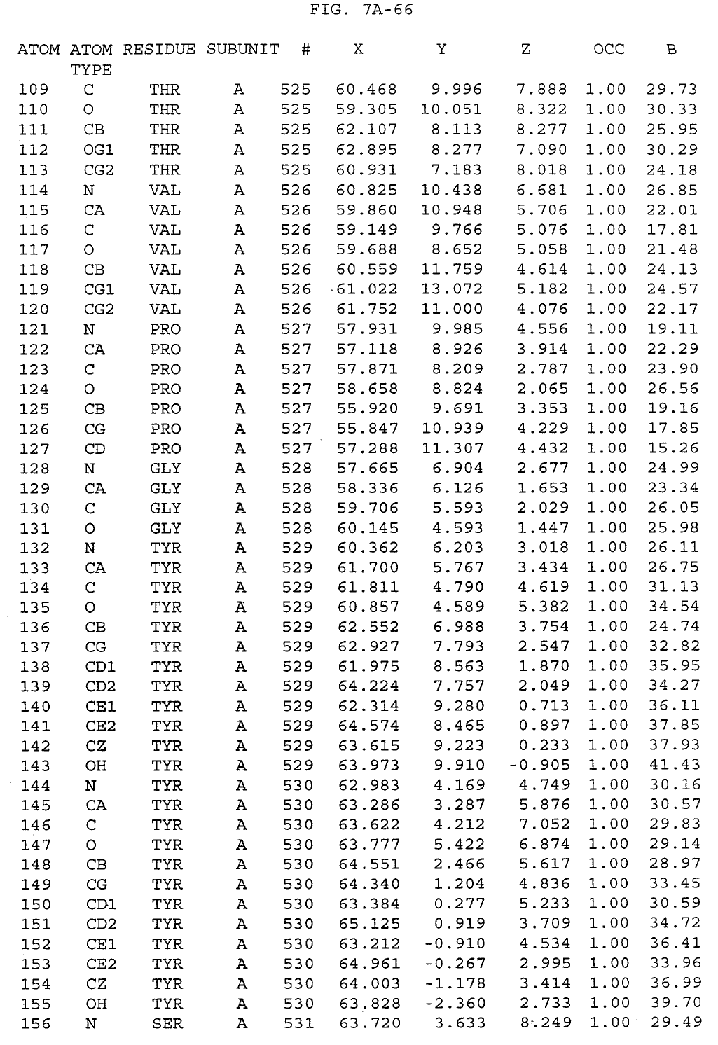

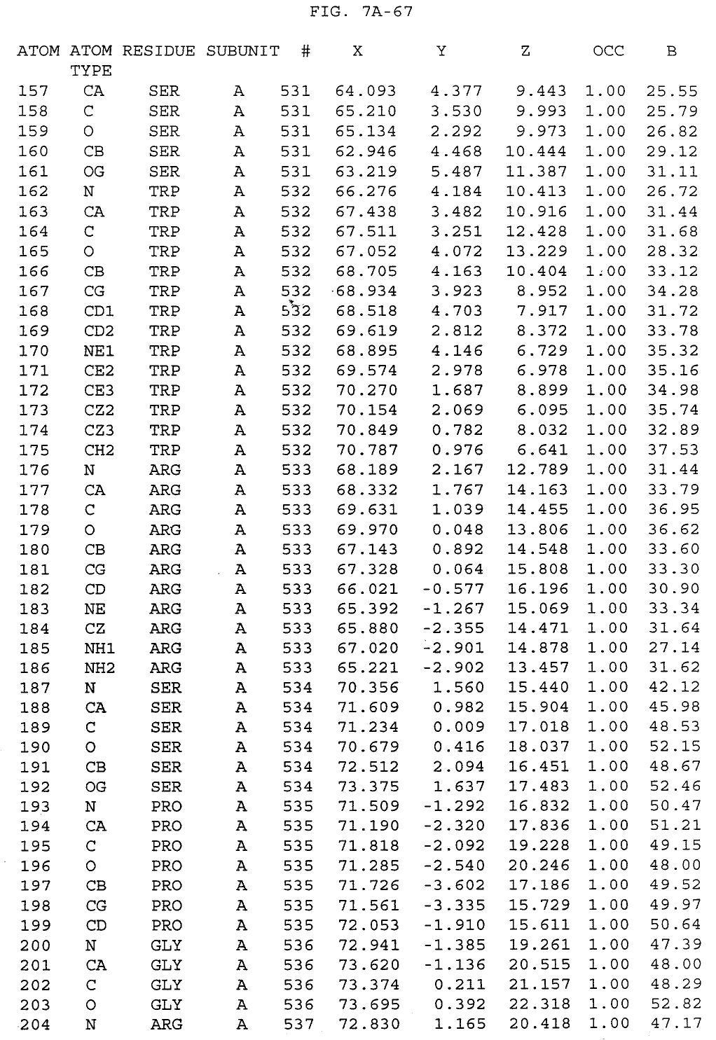

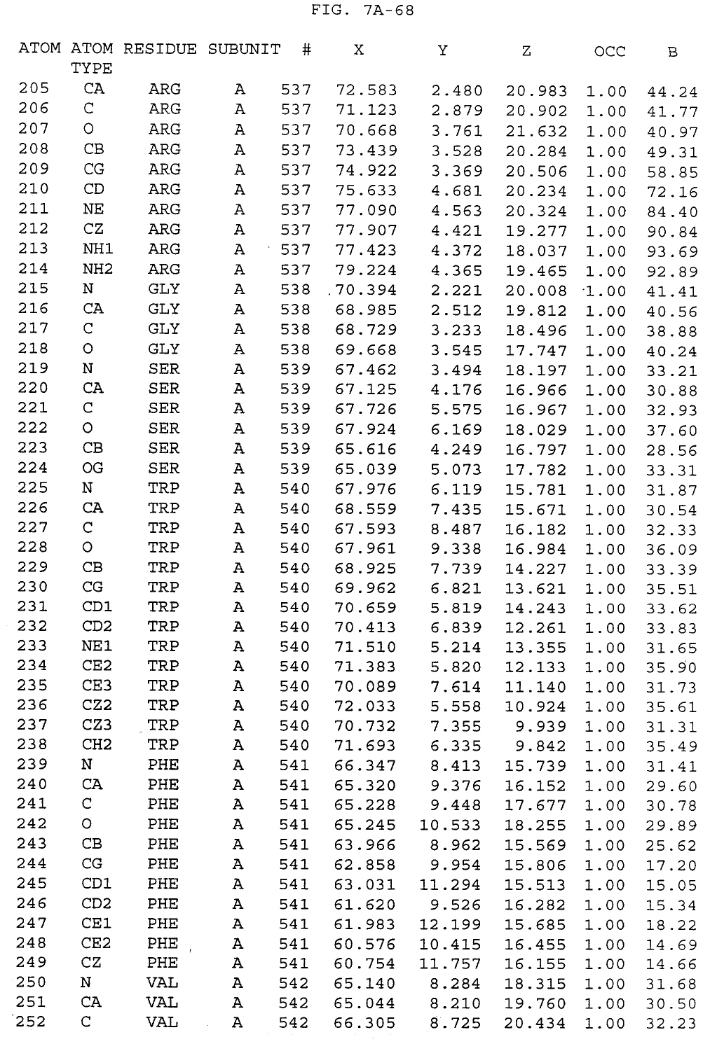

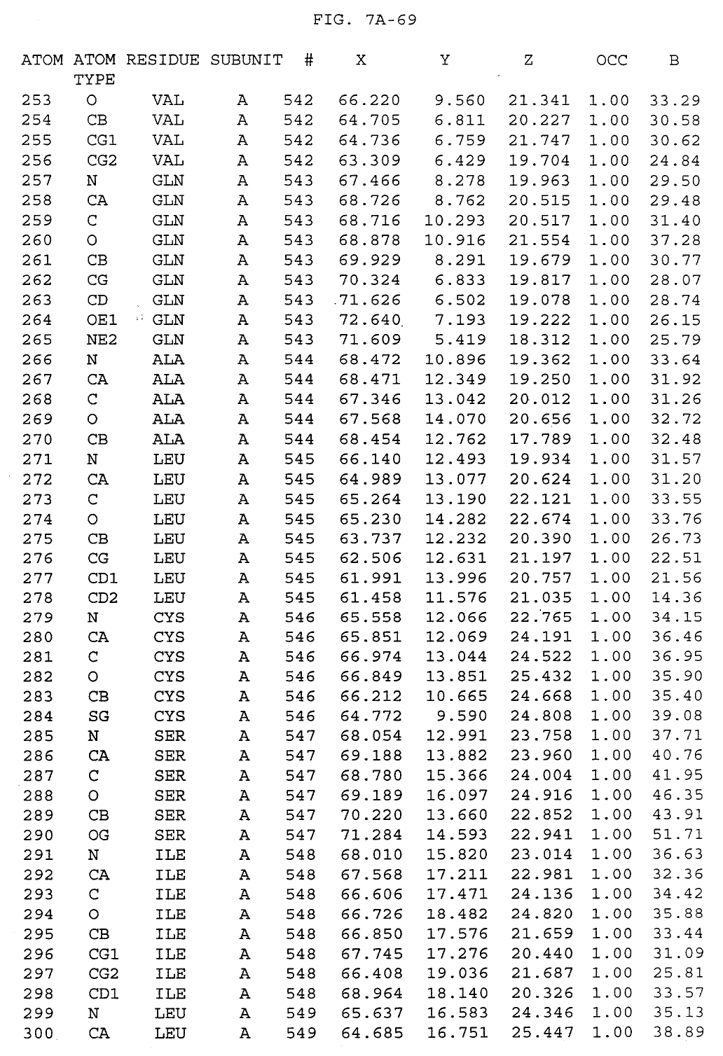

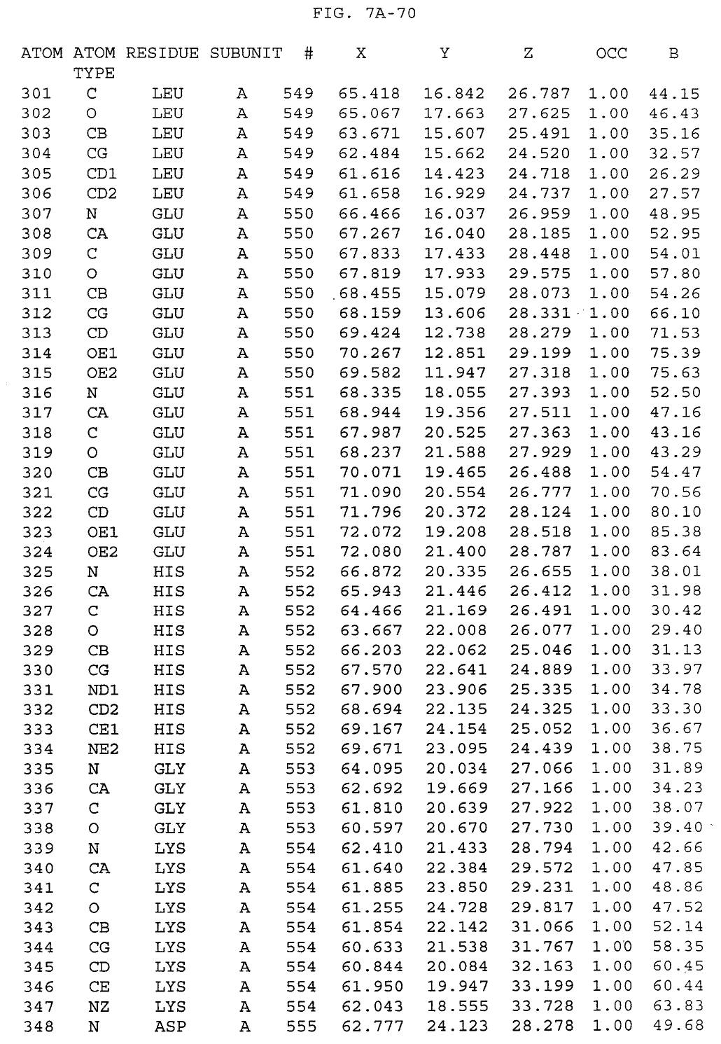

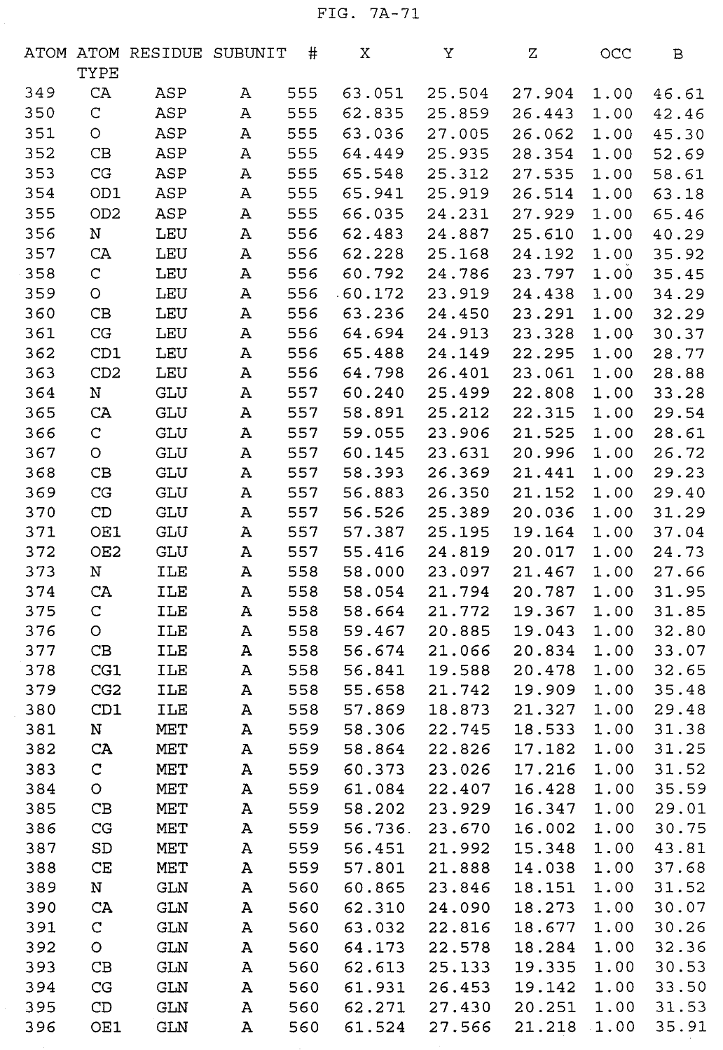

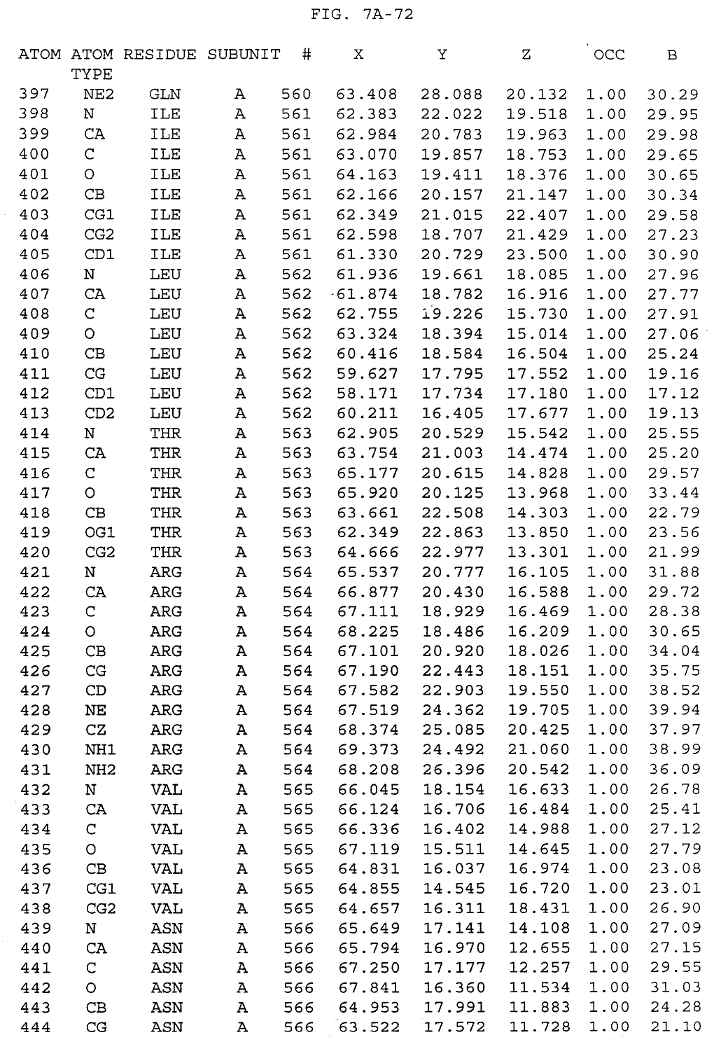

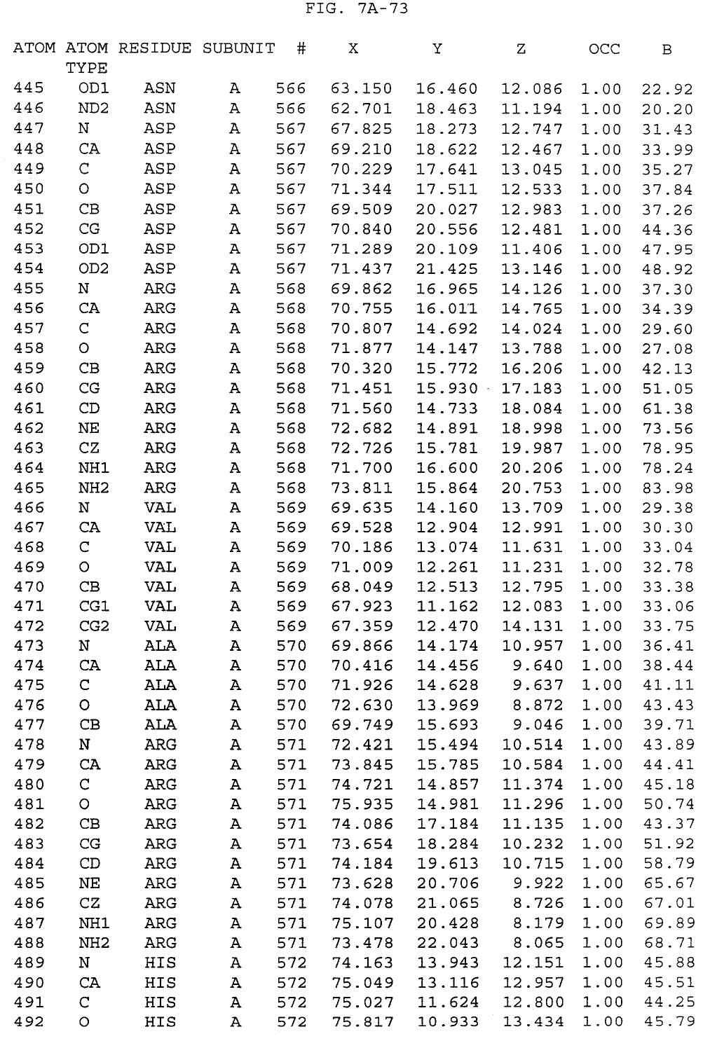

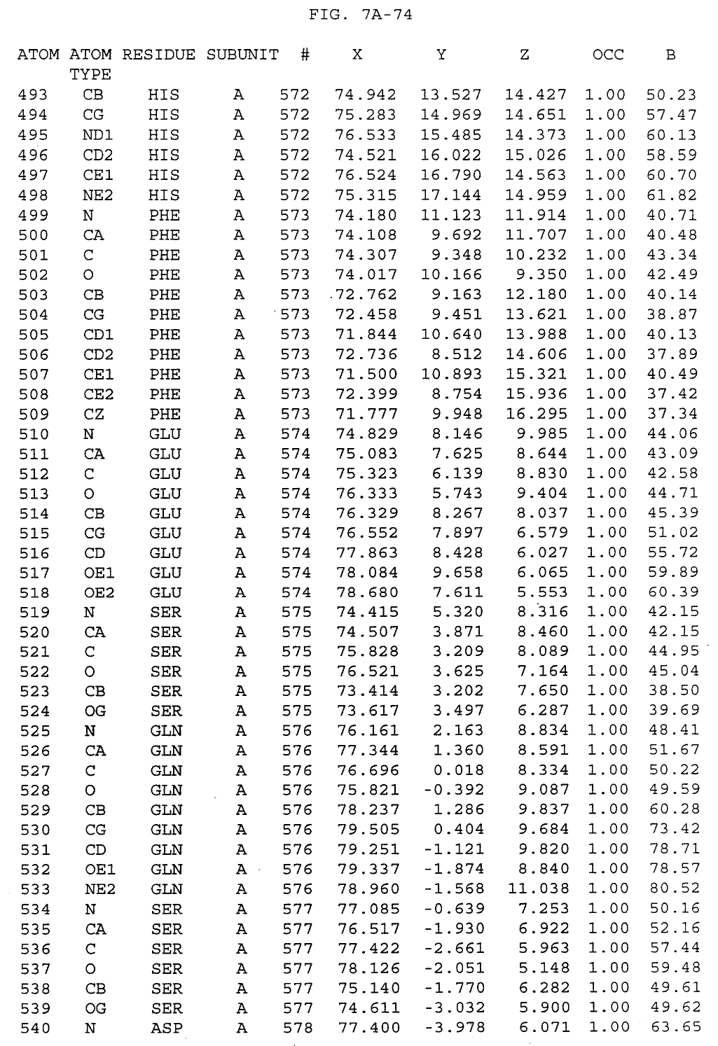

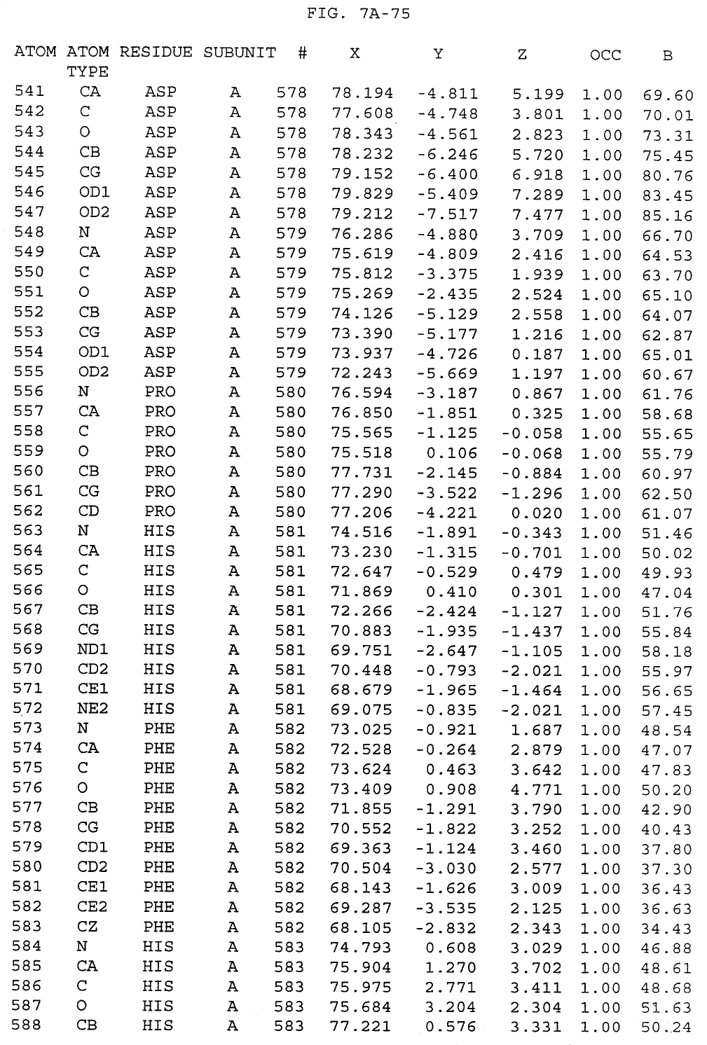

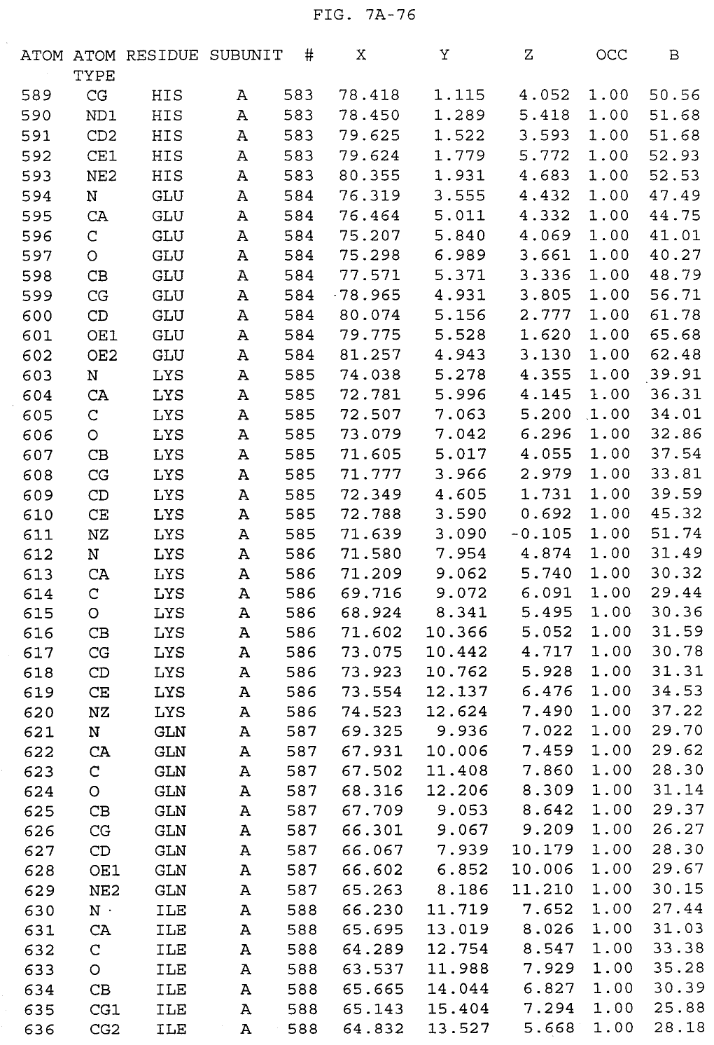

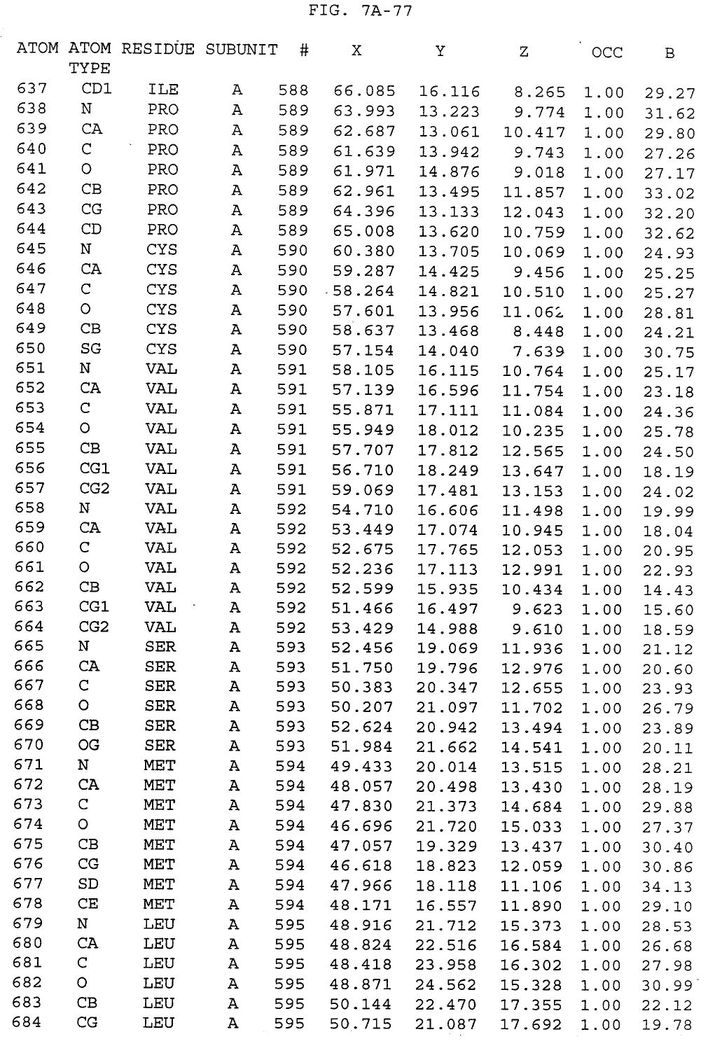

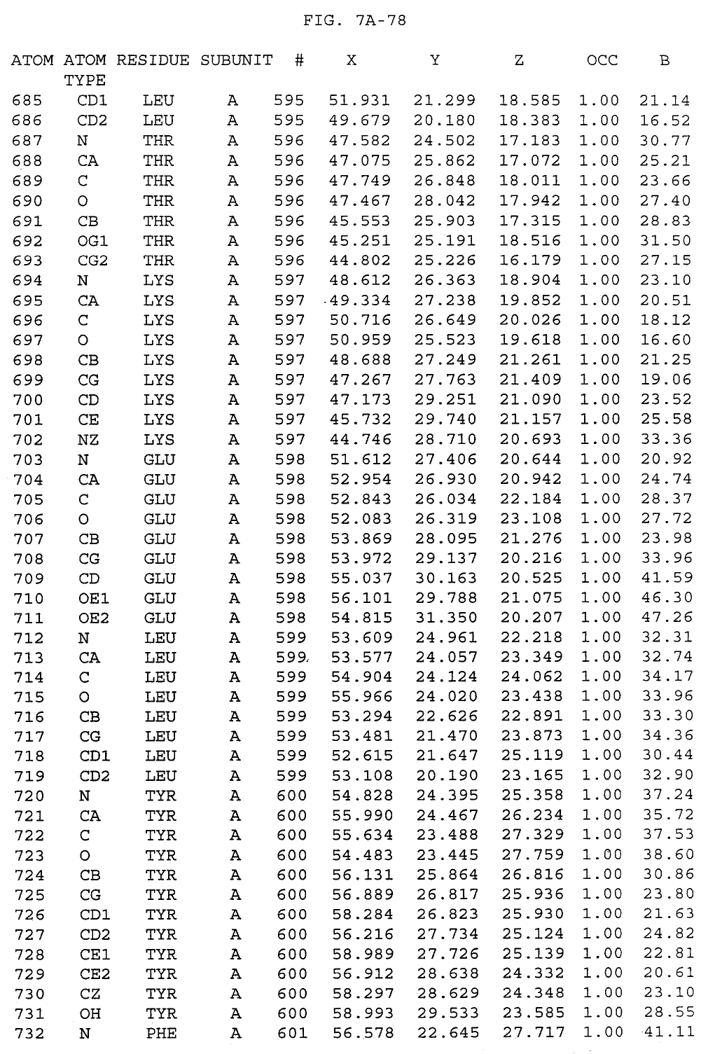

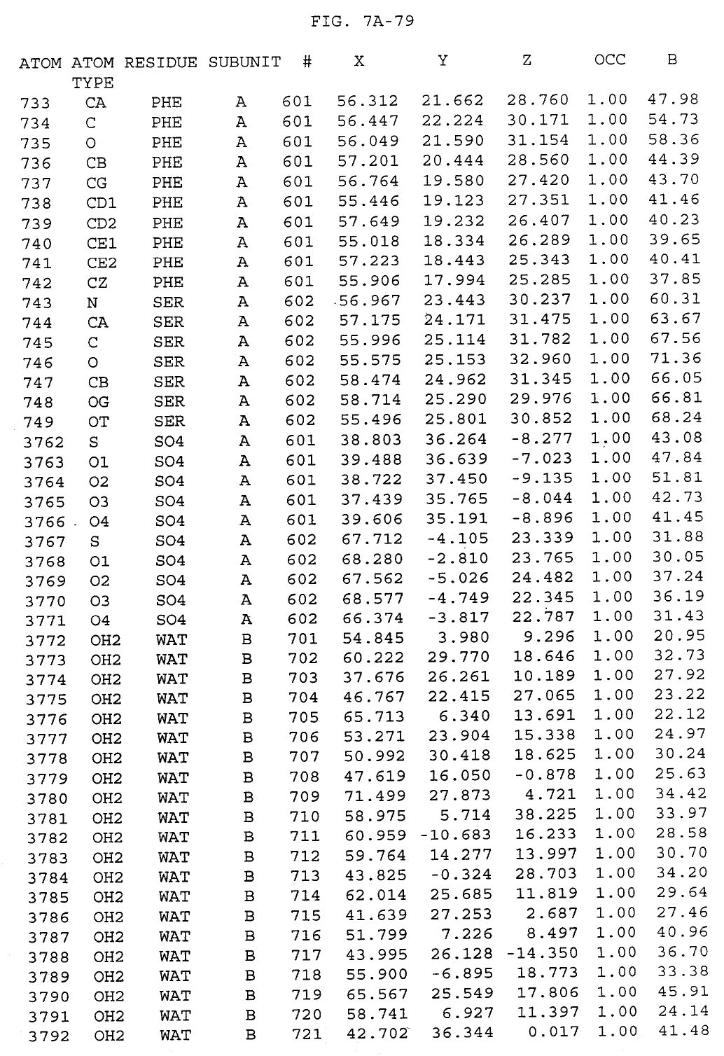

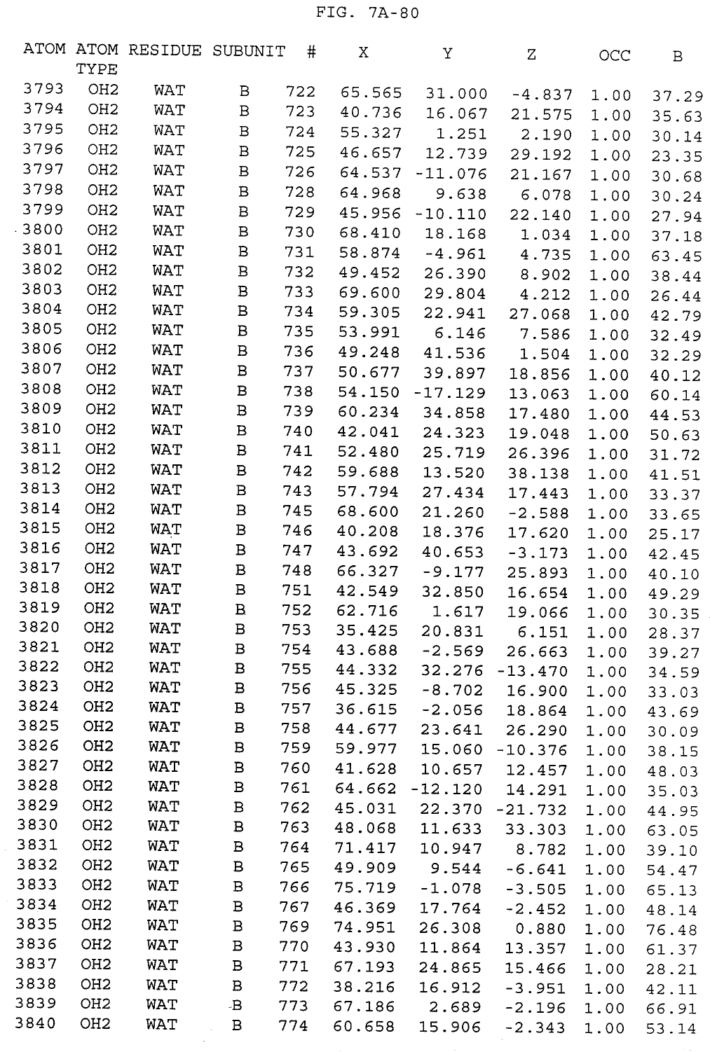

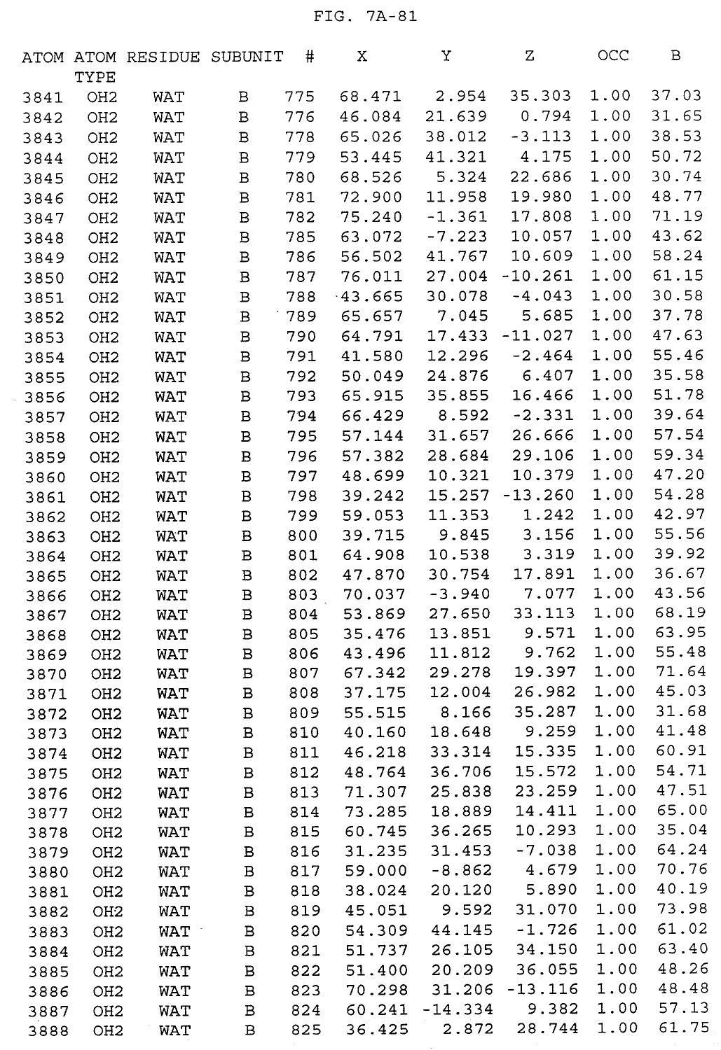

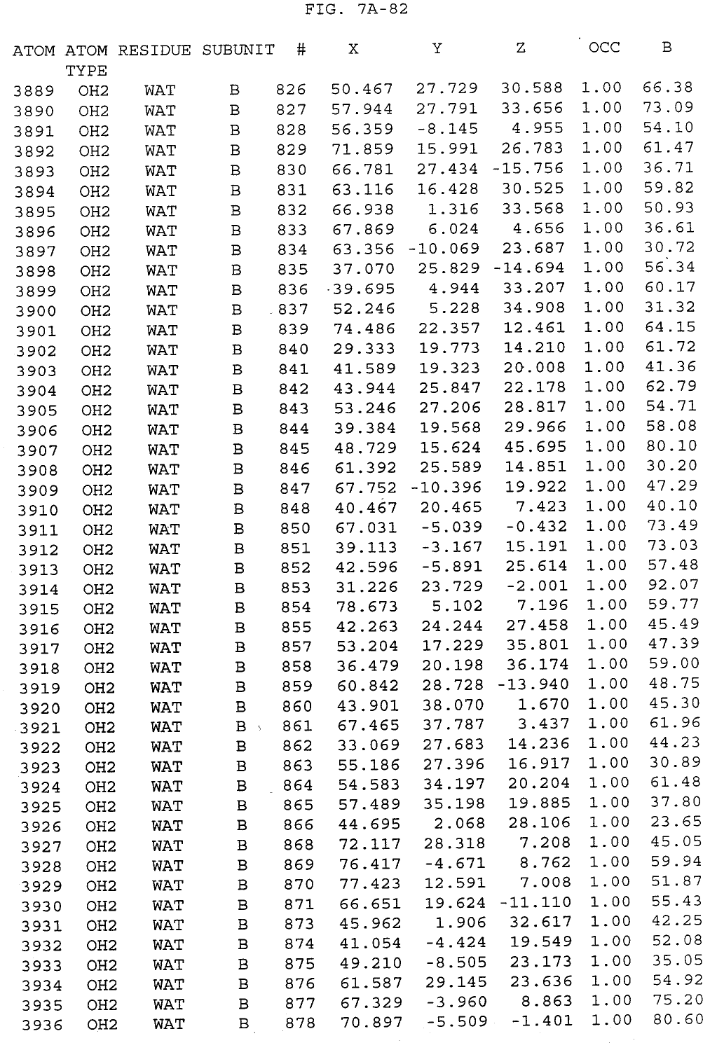

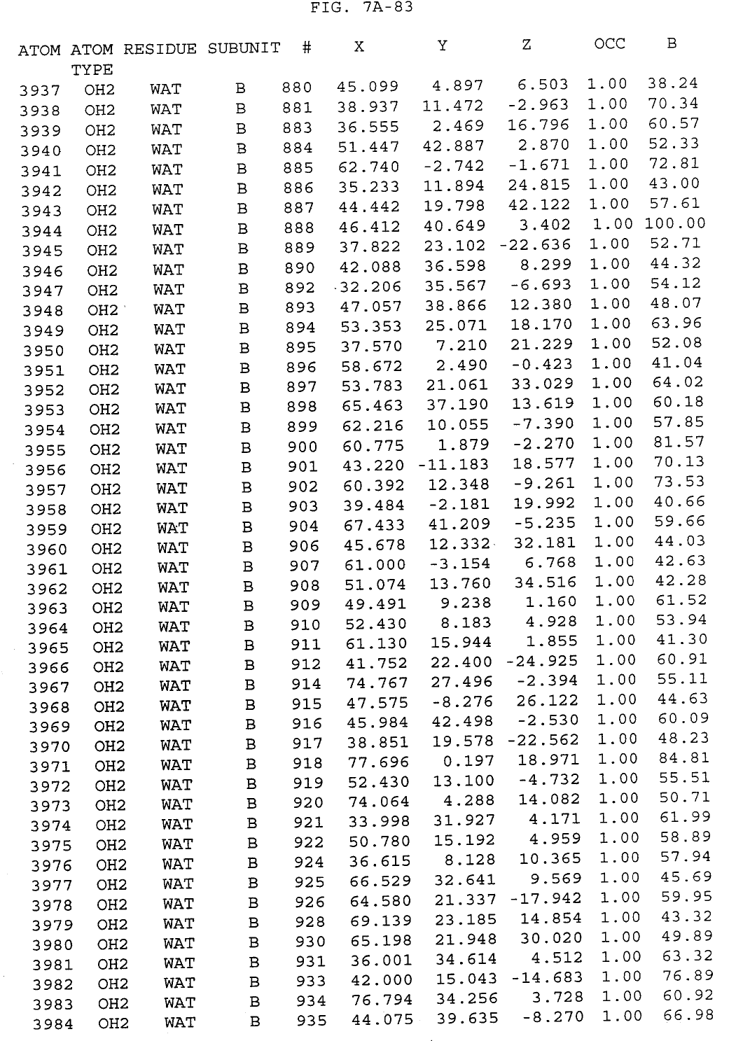

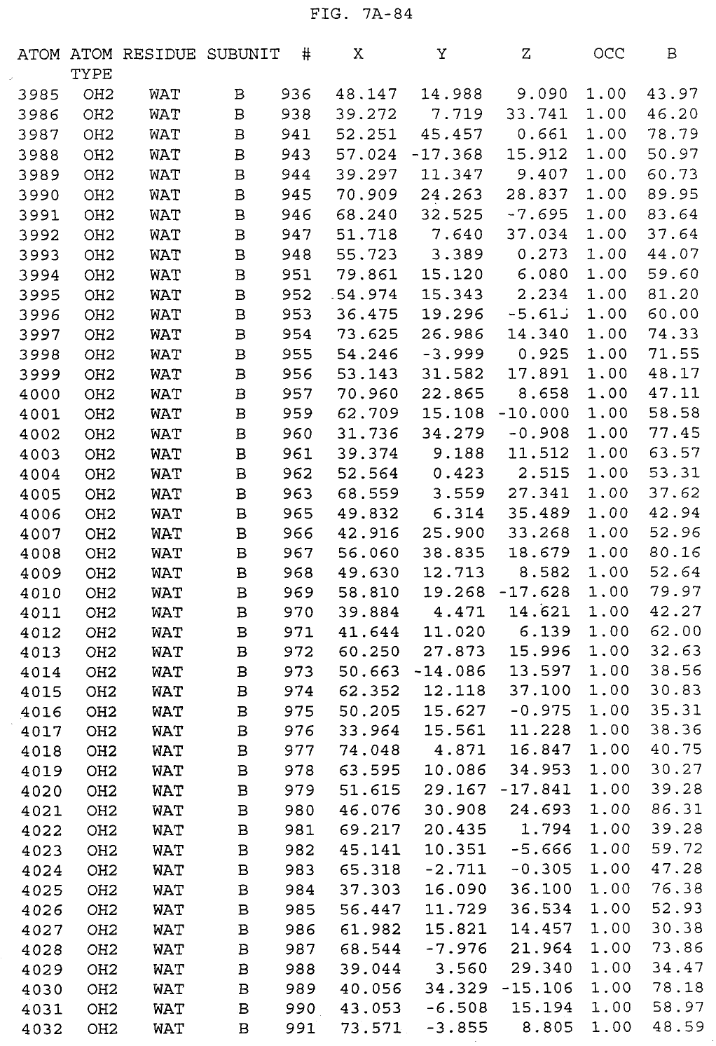

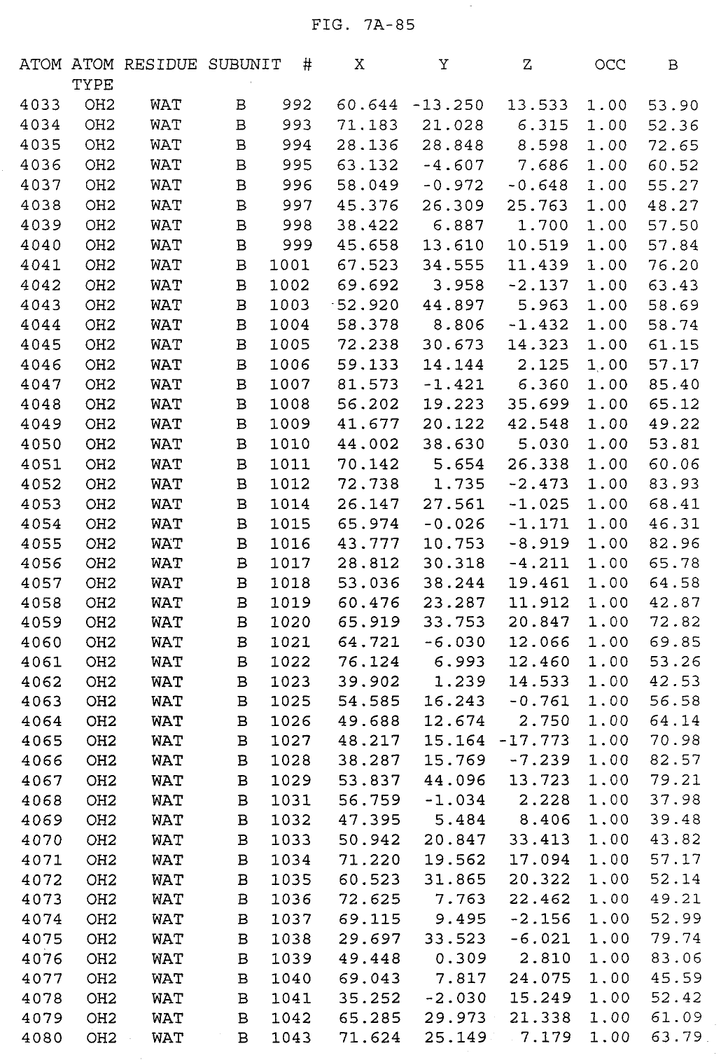

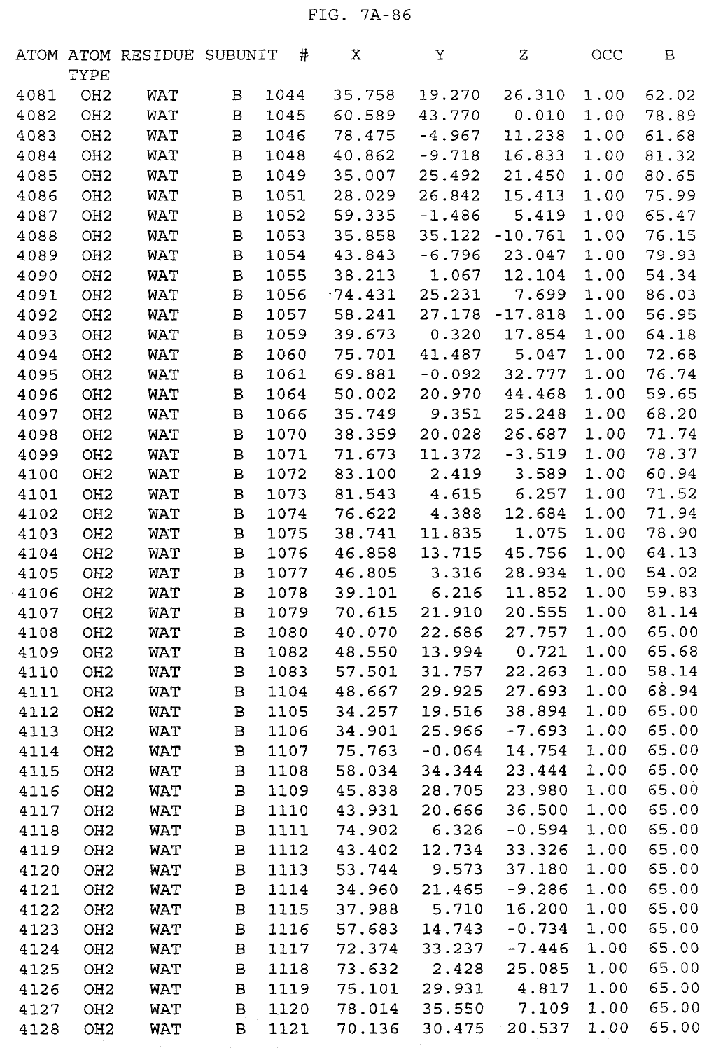

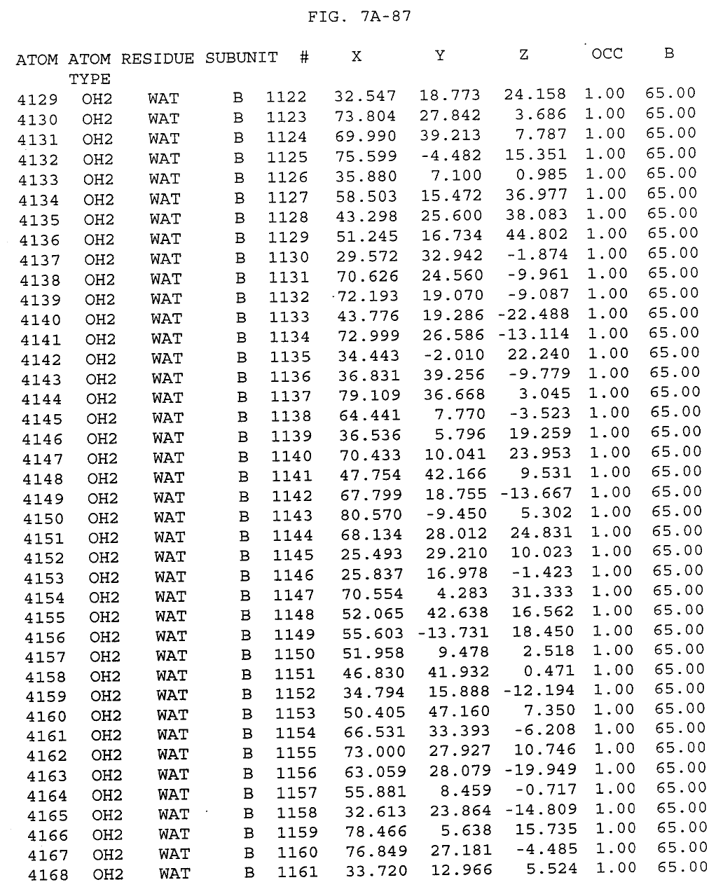

The three-dimensional structure of the caspase-7/inhibitor complex of this invention is defined by a set of structure coordinates as set forth in FIG. 7. The term “structure coordinates” refers to Cartesian coordinates derived from mathematical equations related to the patterns obtained on diffraction of a monochromatic beam of X-rays by the atoms (scattering centers) of a caspase-7/inhibitor complex in crystal form. The diffraction data are used to calculate an electron density map of the repeating unit of the crystal. The electron density maps are then used to establish the positions of the individual atoms of the caspase-7 or caspase-7/inhibitor complex.

Those of skill in the art will understand that a set of structure coordinates for an enzyme or an enzyme-complex or a portion thereof, is a relative set of points that define a shape in three dimensions. Thus, it is possible that an entirely different set of coordinates could define a similar or identical shape. Moreover, slight variations in the individual coordinates will have little effect on overall shape.

The variations in coordinates discussed above may be generated because of mathematical manipulations of the structure coordinates. For example, the structure coordinates set forth in FIG. 7 could be manipulated by crystallographic permutations of the structure coordinates, fractionalization of the structure coordinates, integer additions or subtractions to sets of the structure coordinates, inversion of the structure coordinates or any combination of the above.

Alternatively, modifications in the crystal structure due to mutations, additions, substitutions, and/or deletions of amino acids, or other changes in any of the components that make up the crystal could also account for variations in structure coordinates. If such variations are within an acceptable standard error as compared to the original coordinates, the resulting three-dimensional shape is considered to be the same.

Various computational analyses are therefore necessary to determine whether a molecule or molecular complex or a portion thereof is sufficiently similar to all or parts of the caspase-7/inhbitor structure described above as to be considered the same. Such analyses may be carried out in current software applications, such as the Molecular Similarity application of QUANTA (Molecular Simulations Inc., San Diego, Calif.) version 4.1, and as described in the accompanying User's Guide.

The Molecular Similarity application permits comparisons between different structures, different conformations of the same structure, and different parts of the same structure. The procedure used in Molecular Similarity to compare structures is divided into four steps: 1) load the structures to be compared; 2) define the atom equivalencies in these structures; 3) perform a fitting operation; and 4) analyze the results.

Each structure is identified by a name. One structure is identified as the target (i.e., the fixed structure); all remaining structures are working structures (i.e., moving structures). Since atom equivalency within QUANTA is defined by user input, for the purpose of this invention we will define equivalent atoms as protein backbone atoms (N, Cα, C and O) for all conserved residues between the two structures being compared. We will also consider only rigid fitting operations.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in angstroms, is reported by QUANTA.

For the purpose of this invention, any molecule or molecular complex that has a root mean square deviation of conserved residue backbone atoms (N, Cα, C, O) of less than 1.5 Å when superimposed on the relevant backbone atoms described by structure coordinates listed in FIG. 7 are considered identical. More preferably, the root mean square deviation is less than 1.0 Å.

The term “root mean square deviation” means the square root of the arithmetic mean of the squares of the deviations from the mean. It is a way to express the deviation or variation from a trend or object. For purposes of this invention, the “root mean square deviation” defines the variation in the backbone of a protein or protein complex from the relevant portion of the backbone of the caspase-7 polypeptide portion of the complex as defined by the structure coordinates described herein.

Once the structure coordinates of a protein crystal have been determined they are useful variety of purposes, such as drug discovery and x-ray crystallographic determinations of related proteins.

Thus, in accordance with the present invention, the structure coordinates of a caspase-7 polypeptide/inhibitor complex, and portions thereof is provided. Such data may be used for the purposes of, for example, drug design and molecular replacement.

Accordingly, one embodiment of this invention provides a molecule or molecular complex comprising all or any part of a binding pocket defined by structure coordinates of caspase-7 amino acids 234, 235, 237, 276, 278, 281, and 284 according to FIG. 7, or a homologue of said molecule or molecular complex, wherein said homologue comprises a binding pocket that has a root mean square deviation from the backbone atoms of said amino acids of not more than 1.5 Å, and wherein said molecule or molecular complex has a S4 binding region that is more hydrophilic than the S4 binding region of caspase-3.

Another embodiment of this invention provides a molecule or molecular complex comprising all or any part of a binding pocket defined by structure coordinates of caspase-7 amino acids 85, 86, 87, 88, 144, 145, 184, 186, 191, 223, 230, 231, 232, 233, 234, 235, 237, 240, 276, 278, 281, 282, and 284 or a homologue of said molecule or molecular complex, wherein said homologue comprises a binding pocket that has a root mean square deviation from the backbone atoms of said amino acids of not more than 1.5 Å, and wherein said molecule or molecular complex has a S4 binding region that is more hydrophilic than the S4 binding region of caspase-3.

Yet another embodiment of this invention provides a molecule or molecular complex defined by all or part of the structure coordinates according to FIG. 7, or a homologue thereof, wherein said homologue has a root mean square deviation from the conserved backbone atoms of said amino acids of not more than 1.5 Å, and wherein said molecule or molecular complex has a S4 binding region that is more hydrophilic than the S4 binding region of caspase-3. Preferably, a molecule or molecular complex is defined by all of the structure coordinates according to FIG. 7 or a homologue thereof.

Yet another embodiment of this invention provides a molecule or molecular complex defined by all or part of the structure coordinates of caspase-7 amino acids 58-302 according to FIG. 7, or a homologue thereof, wherein said homologue has a root mean square deviation from the conserved backbone atoms of said amino acids of not more than 1.5 Å, and wherein said molecule or molecular complex has a S4 binding region that is more hydrophilic than the S4 binding region of caspase-3. Preferably, the molecule or molecular complex is defined by all of the structure coordinates of caspase-7 amino acids 58-302 according to FIG. 7 or a homologue thereof.

Preferably, in any of these embodiments, the homologue has a root mean square deviation from the conserved backbone atoms of said amino acids of not more than 1.0 Å.

In a more preferred embodiment, the molecule or molecular complex is defined by the structure coordinates of caspase-7 amino acids 58-302 according to FIG. 7.

This invention also provides a machine-readable data storage medium, comprising a data storage material encoded with machine readable data, wherein said data is defined by the all or a portion of the structure coordinates of a caspase-7 complex according to FIG. 7, or a homologue of said complex, wherein said homologue comprises backbone atoms that have a root mean square deviation from the backbone atoms of the complex of not more than 1.5 Å.

Preferably, the data is defined by the structure coordinates of caspase-7 amino acids 234, 235, 237, 276, 278, 281, and 284 according to FIG. 7, or a homologue of said complex, wherein said homologue comprises backbone atoms that have a root mean square deviation from the backbone atoms of the complex of not more than 1.5 Å.

More preferably, the data is defined by the structure coordinates of caspase-7 amino acids 85, 86, 87, 88, 144, 145, 184, 186, 191, 223, 230, 231, 232, 233, 234, 235, 237, 240, 276, 278, 281, 282, and 284 according to FIG. 7, or a homologue of said complex, wherein said homologue comprises backbone atoms that have a root mean square deviation from the backbone atoms of the complex of not more than 1.5 Å.

Even more preferably, the data is defined by the structure coordinates for caspase-7 amino acids 58-302 according to FIG. 7, or a homologue of said molecule or molecular complex, said homologue having a root mean square deviation from the backbone atoms of said amino acids of not more than 1.5 Å.

Preferably, in any of these embodiments, the homologue has a root mean square deviation from the conserved backbone atoms of said amino acids of not more than 1.0 Å.

More preferably, the data is defined by the structure coordinates for caspase-7 amino acids 58-302 according to FIG. 7.

According to this invention, these caspase-7 complexes and homologues thereof have a S4 binding region that is more hydrophilic than the S4 binding region of caspase-3.

To use the structure coordinates generated for the caspase-7 complex or one of its binding pockets or homologues thereof, it is sometimes necessary to convert them into a three-dimensional shape. This is achieved through the use of commercially available software that is capable of a generating a three-dimensional representation of molecules or portions thereof from a set of structure coordinates. The three-dimensional representations may be displayed as a graphical representation.

Therefore, according to another embodiment of this invention is provided a machine-readable data storage medium, comprising a data storage material encoded with machine readable data, when using a machine programmed with instructions for using said data, is capable of producing a three-dimensional representation of any of the molecule or molecular complexes, or homologues thereof, that are described herein.

This invention also provides a computer for producing a three-dimensional representation of:

a. a molecule or molecular complex comprising a binding pocket defined by structure coordinates of caspase-7 amino acids 234, 235, 237, 276, 278, 281, and 284 according to FIG. 7; or

b. a homologue of said molecule or molecular complex, wherein said homologue comprises a binding pocket that has a root mean square deviation from the backbone atoms of said amino acids of not more than 1.5 Å, wherein said computer comprises:

i. a machine-readable data storage medium comprising a data storage material encoded with machine-readable data, wherein said data comprises the structure coordinates of caspase-7 amino acids 234, 235, 237, 276, 278, 281, and 284 according to FIG. 7;

ii. instructions for processing said machine-readable data into said three-dimensional representation.

Preferably, the computer is for producing a three-dimensional representation of a molecule or molecular complex defined by the set of structure coordinates for caspase-7 amino acids 85, 86, 87, 88, 144, 145, 184, 186, 191, 223, 230, 231, 232, 233, 234, 235, 237, 240, 276, 278, 281, 282, and 284 according to FIG. 7, or a three-dimensional representation is of a homologue of said molecule or molecular complex, said homologue having a root mean square deviation from the backbone atoms of said amino acids of not more than 1.5 Å.

More preferably, the computer is for producing a three-dimensional representation of a molecule or molecular complex defined by all or a portion of the set of structure coordinates according to FIG. 7, or a homologue of said molecule or molecular complex, said homologue having a root mean square deviation from the backbone atoms of said amino acids of not more than 1.5 Å.

Most preferably, the computer is for producing a three-dimensional defined by the set of structure coordinates of caspase-7 amino acids 58-302 according to FIG. 7, or a homologue of said molecule or molecular complex, or a homologue thereof having a root mean square deviation from the backbone atoms of said amino acids of not more than 1.5 Å.

Preferably, in any of these embodiments, the homologue has a root mean square deviation from the conserved backbone atoms of said amino acids of not more than 1.0 Å.

Most preferably, the computer is for producing a three-dimensional defined by the set of structure coordinates of caspase-7 amino acids 58-302 according to FIG. 7.

In a preferred embodiment, a computer according to this invention also comprises a working memory for storing instructions for processing the machine-readable data, a central-processing unit coupled to the working memory and to said machine-readable data storage medium for processing said machine-readable data into the three-dimensional representation, or a display for displaying the three-dimensional representation. More preferably, a computer according to this invention comprises a display. Most preferably, a computer according to this invention comprises the above working memory, central-processing unit, and display.

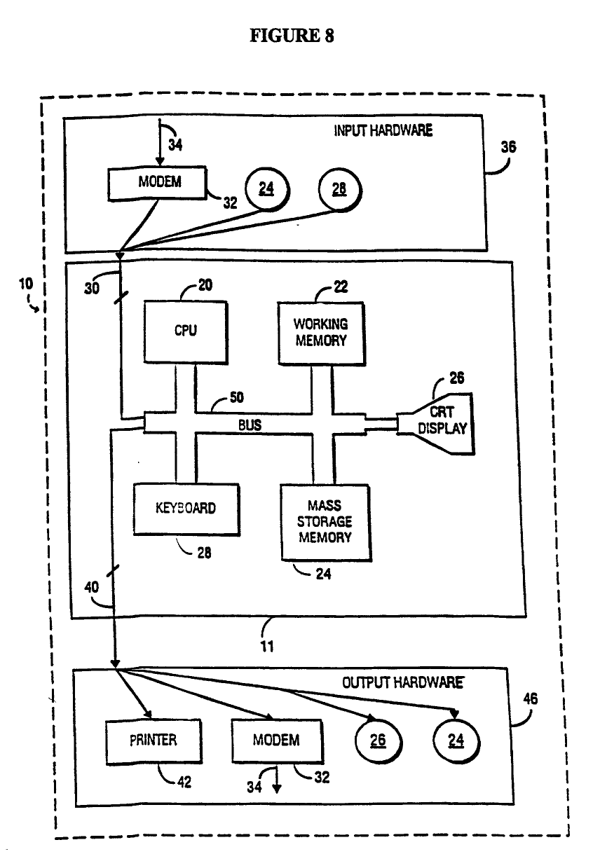

FIG. 8 demonstrates one version of these embodiments. System 10 includes a computer 11 comprising a central processing unit (“CPU”) 20, a working memory 22 which may be, e.g., RAM (random-access memory) or “core” memory, mass storage memory 24 (such as one or more disk drives or CD-ROM drives), one or more cathode-ray tube (“CRT”) display terminals 26, one or more keyboards 28, one or more input lines 30, and one or more output lines 40, all of which are interconnected by a conventional bidirectional system bus 50.

Input hardware 36, coupled to computer 11 by input lines 30, may be implemented in a variety of ways. Machine-readable data of this invention may be inputted via the use of a modem or modems 32 connected by a telephone line or dedicated data line 34. Alternatively or additionally, the input hardware 36 may comprise CD-ROM drives or disk drives 24. In conjunction with display terminal 26, keyboard 28 may also be used as an input device.

Output hardware 46, coupled to computer 11 by output lines 40, may similarly be implemented by conventional devices. By way of example, output hardware 46 may include CRT display terminal 26 for displaying a graphical representation of a binding pocket of this invention using a program such as QUANTA as described herein. Output hardware might also include a printer 42, so that hard copy output may be produced, or a disk drive 24, to store system output for later use.

In operation, CPU 20 coordinates the use of the various input and output devices 36, 46, coordinates data accesses from mass storage 24 and accesses to and from working memory 22, and determines the sequence of data processing steps. A number of programs may be used to process the machine-readable data of this invention. Such programs are discussed in reference to the computational methods of drug discovery as described herein. Specific references to components of the hardware system 10 are included as appropriate throughout the following description of the data storage medium.

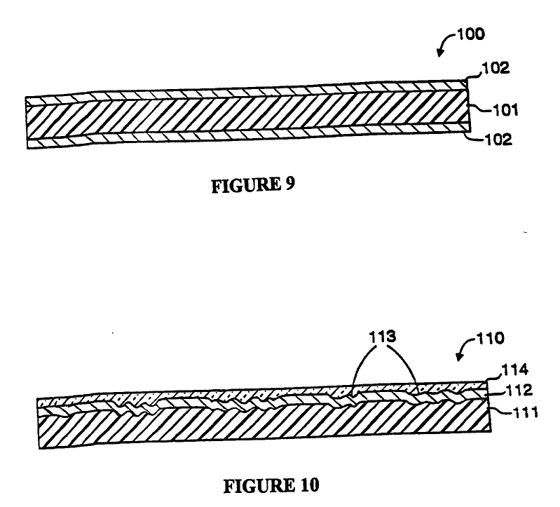

FIG. 9 shows a cross section of a magnetic data storage medium 100 which can be encoded with a machine-readable data that can be carried out by a system such as system 10 of FIG. 8. Medium 100 can be a conventional floppy diskette or hard disk, having a suitable substrate 101, which may be conventional, and a suitable coating 102, which may be conventional, on one or both sides, containing magnetic domains (not visible) whose polarity or orientation can be altered magnetically. Medium 100 may also have an opening (not shown) for receiving the spindle of a disk drive or other data storage device 24.

The magnetic domains of coating 102 of medium 100 are polarized or oriented so as to encode in manner which may be conventional, machine readable data such as that described herein, for execution by a system such as system 10 of FIG. 8.

FIG. 10 shows a cross section of an optically-readable data storage medium 110 which also can be encoded with such a machine-readable data, or set of instructions, which can be carried out by a system such as system 10 of FIG. 8. Medium 110 can be a conventional compact disk read only memory (CD-ROM) or a rewritable medium such as a magneto-optical disk which is optically readable and magneto-optically writable. Medium 100 preferably has a suitable substrate 111, which may be conventional, and a suitable coating 112, which may be conventional, usually of one side of substrate 111.

In the case of CD-ROM, as is well known, coating 112 is reflective and is impressed with a plurality of pits 113 to encode the machine-readable data. The arrangement of pits is read by reflecting laser light off the surface of coating 112. A protective coating 114, which preferably is substantially transparent, is provided on top of coating 112.

In the case of a magneto-optical disk, as is well known, coating 112 has no pits 113, but has a plurality of magnetic domains whose polarity or orientation can be changed magnetically when heated above a certain temperature, as by a laser (not shown). The orientation of the domains can be read by measuring the polarization of laser light reflected from coating 112. The arrangement of the domains encodes the data as described above.

For the first time, the present invention permits the use of drug discovery techniques, including structure-based, rational drug design, or database screening techniques, to design, select, and/or synthesize chemical entities, including inhibitory compounds that are capable of binding to caspase-7, or any portion thereof.

For example, the structure encoded by the data may be computationally evaluated for its ability to associate with chemical entities. Chemical entities that associate with caspase-7 may inhibit caspase-7, and are potential drug candidates. Alternatively, the structure encoded by the data may be displayed in a graphical three-dimensional representation on a computer screen. This allows visual inspection of the structure, as well as visual inspection of the structure's association with chemical entities.

Thus, according to another embodiment, the invention relates to a method for evaluating the potential or ability of a chemical entity to associate with any of the molecules or molecular complexes set forth above. This method comprises the steps of: a) employing computational means to perform a fitting operation between the chemical entity and a binding pocket of the molecule or molecular complex; and b) analyzing the results of said fitting operation to quantify the association between the chemical entity and the binding pocket. The term “chemical entity”, as used herein, refers to chemical compounds, complexes of at least two chemical compounds, and fragments of such compounds or complexes.

For the first time, the present invention permits the use of molecular design techniques to identify, select and design chemical entities, including inhibitory compounds, capable of binding to caspase-7-like binding pockets.

Applicants' elucidation of the caspase-7 binding sites provides the necessary information for designing new chemical entities and compounds that may interact with the caspase-7 binding pockets, in whole or in part. This elucidation also enables the evaluation of structure-activity data for compounds which bind to caspase-7 binding pockets. Applicants' elucidation of the caspase-7 binding sites also provides important structural information for comparing the requirements for interacting with caspase-7 binding pockets selectively or in addition to other caspase binding pockets.

Thus, according to another embodiment, the invention relates to a method for a method for identifying a potential agonist or antagonist of any of the molecules or molecular complexes set forth above. This method comprises the steps of: a. generating a three-dimensional structure of the molecule or molecular complex; b. employing said three-dimensional structure to design or select said potential agonist or antagonist; c. providing said potential agonist or antagonist; and d. contacting said potential agonist or antagonist to interact with said molecule.

Throughout this section, discussions about the ability of an entity to bind to, associate with or inhibit a caspase-7 binding pocket refers to features of the entity alone. Assays to determine whether a compound binds to caspases are well known in the art [see for example, the assays described in WO97/22619].

The design of compounds that bind to or inhibit caspase-7 according to this invention generally involves consideration of two factors. First, the entity must be capable of physically and structurally associating with parts or all of the caspase-7 binding pockets. Non-covalent molecular interactions important in this association include hydrogen bonding, van der Waals interactions, hydrophobic interactions and electrostatic interactions.

Second, the entity must be able to assume a conformation that allows it to associate with the caspase-7 binding pocket directly. Although certain portions of the entity will not directly participate in these associations, those portions of the entity may still influence the overall conformation of the molecule. This, in turn, may have a significant impact on potency. Such conformational requirements include the overall three-dimensional structure and orientation of the chemical entity in relation to all or a portion of the binding pocket, or the spacing between functional groups of an entity comprising several chemical entities that directly interact with the caspase-7 binding pocket or homologues thereof.

The potential inhibitory or binding effect of a chemical entity on a caspase-7 binding pocket may be analyzed prior to its actual synthesis and testing by the use of computer modeling techniques. If the theoretical structure of the given entity suggests insufficient interaction and association between it and the caspase-7 binding pocket, testing of the entity is obviated. However, if computer modeling indicates a strong interaction, the molecule may then be provided or synthesized and tested for its ability to bind to a caspase-7 binding pocket. This may be achieved by testing the ability of the molecule to inhibit caspase-7 using assays that are known to those of ordinary skill in the art. In this manner, the obtaining and testing of inoperative compounds may be avoided.

A potential inhibitor of caspase-7 may be computationally evaluated by means of a series of steps in which chemical entities or fragments are screened and selected for their ability to associate with the caspase-7 binding pockets.

One skilled in the art may use one of several methods to screen chemical entities or fragments for their ability to associate with a caspase-7 binding pocket. This process may begin by visual inspection of, for example, a caspase-7 binding pocket on the computer screen based on the caspase-7 structure coordinates in FIG. 7 or other coordinates which define a similar shape generated from the machine-readable storage medium. Selected fragments or chemical entities may then be positioned in a variety of orientations, or docked, within that binding pocket as defined supra. Docking may be accomplished using software such as Quanta and Sybyl, followed by energy minimization and molecular dynamics with standard molecular mechanics force fields, such as CHARMM and AMBER.

Specialized computer programs may also assist in the process of selecting fragments or chemical entities. These include:

1. GRID (P. J. Goodford, “A Computational Procedure for Determining Energetically Favorable Binding Sites on Biologically Important Macromolecules”, J. Med. Chem., 28, pp. 849-857 (1985)). GRID is available from Oxford University, Oxford, UK.

2. MCSS (A. Miranker et al., “Functionality Maps of Binding Sites: A Multiple Copy Simultaneous Search Method.” Proteins: Structure, Function and Genetics, 11, pp. 29-34 (1991)). MCSS is available from Molecular Simulations, San Diego, Calif.

3. AUTODOCK (D. S. Goodsell et al., “Automated Docking of Substrates to Proteins by Simulated Annealing”, Proteins: Structure, Function, and Genetics, 8, pp. 195-202 (1990)). AUTODOCK is available from Scripps Research Institute, La Jolla, Calif.

4. DOCK (I. D. Kuntz et al., “A Geometric Approach to Macromolecule-Ligand Interactions”, J. Mol. Biol., 161, pp. 269-288 (1982)). DOCK is available from University of California, San Francisco, Calif.

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of caspase-7. This would be followed by manual model building using software such as Quanta or Sybyl [Tripos Associates, St. Louis, Mo.].

Useful programs to aid one of skill in the art in connecting the individual chemical entities or fragments include:

1. CAVEAT (P. A. Bartlett et al., “CAVEAT: A Program to Facilitate the Structure-Derived Design of Biologically Active Molecules”, in Molecular Recognition in Chemical and Biological Problems”, Special Pub., Royal Chem. Soc., 78, pp. 182-196 (1989); G. Lauri and P. A. Bartlett, “CAVEAT: a Program to Facilitate the Design of Organic Molecules”, J. Comput. Aided Mol. Des., 8, pp. 51-66 (1994)). CAVEAT is available from the University of California, Berkeley, Calif.

2. 3D Database systems such as ISIS (MDL Information Systems, San Leandro, Calif.). This area is reviewed in Y. C. Martin, “3D Database Searching in Drug Design”, J. Med. Chem., 35, pp. 2145-2154 (1992).

3. HOOK (M. B. Eisen et al., “HOOK: A Program for Finding Novel Molecular Architectures that Satisfy the Chemical and Steric Requirements of a Macromolecule Binding Site”, Proteins: Struct., Funct., Genet., 19, pp. 199-221 (1994). HOOK is available from Molecular Simulations, San Diego, Calif.

Instead of proceeding to build an inhibitor of a caspase-7 binding pocket in a step-wise fashion one fragment or chemical entity at a time as described above, inhibitory or other caspase-7 binding compounds may be designed as a whole or “de novo” using either an empty binding site or optionally including some portion(s) of a known inhibitor(s). There are many de novo ligand design methods including:

1. LUDI (H.-J. Bohm, “The Computer Program LUDI: A New Method for the De Novo Design of Enzyme Inhibitors”, J. Comp. Aid. Molec. Design, 6, pp. 61-78 (1992)). LUDI is available from Molecular Simulations Incorporated, San Diego, Calif.

2. LEGEND (Y. Nishibata et al., Tetrahedron, 47, p. 8985 (1991)). LEGEND is available from Molecular Simulations Incorporated, San Diego, Calif.

3. LeapFrog (available from Tripos Associates, St. Louis, Mo.).

4. SPROUT (V. Gillet et al., “SPROUT: A Program for Structure Generation)”, J. Comput. Aided Mol. Design, 7, pp. 127-153 (1993)). SPROUT is available from the University of Leeds, UK.

Other molecular modeling techniques may also be employed in accordance with this invention [see, e.g., N.C. Cohen et al., “Molecular Modeling Software and Methods for Medicinal Chemistry, J. Med. Chem., 33, pp. 883-894 (1990); see also, M. A. Navia and M. A. Murcko, “The Use of Structural Information in Drug Design”, Current Opinions in Structural Biology, 2, pp. 202-210 (1992); L. M. Balbes et al., “A Perspective of Modern Methods in Computer-Aided Drug Design”, in Reviews in Computational Chemistry, Vol. 5, K. B. Lipkowitz and D. B. Boyd, Eds., VCH, New York, pp. 337-380 (1994); see also, W. C. Guida, “Software For Structure-Based Drug Design”, Curr. Opin. Struct. Biology, 4, pp. 777-781 (1994)].

Once a compound has been designed or selected by the above methods, the efficiency with which that entity may bind to a caspase-7 binding pocket may be tested and optimized by computational evaluation. For example, an effective caspase-7 binding pocket inhibitor must preferably demonstrate a relatively small difference in energy between its bound and free states (i.e., a small deformation energy of binding). Thus, the most efficient caspase-7 binding pocket inhibitors should preferably be designed with a deformation energy of binding of not greater than about 10 kcal/mole, more preferably, not greater than 7 kcal/mole. Caspase-7 binding pocket inhibitors may interact with the binding pocket in more than one conformation that is similar in overall binding energy. In those cases, the deformation energy of binding is taken to be the difference between the energy of the free entity and the average energy of the conformations observed when the inhibitor binds to the protein.

An entity designed or selected as binding to a caspase-7 binding pocket may be further computationally optimized so that in its bound state it would preferably lack repulsive electrostatic interaction with the target enzyme and with the surrounding water molecules. Such non-complementary electrostatic interactions include repulsive charge-charge, dipole-dipole, and charge-dipole interactions.

Specific computer software is available in the art to evaluate compound deformation energy and electrostatic interactions. Examples of programs designed for such uses include: Gaussian 94, revision C (M. J. Frisch, Gaussian, Inc., Pittsburgh, Pa. @1995); AMBER, version 4.1 (P. A. Kollman, University of California at San Francisco, @1995); QUANTA/CHARMM (Molecular Simulations, Inc., San Diego, Calif. @1995); Insight II/Discover (Molecular Simulations, Inc., San Diego, Calif. @1995); DelPhi (Molecular Simulations, Inc., San Diego, Calif. @1995); and AMSOL (Quantum Chemistry Program Exchange, Indiana University). These programs may be implemented, for instance, using a Silicon Graphics workstation such as an Indigo² with “IMPACT” graphics. Other hardware systems and software packages will be known to those skilled in the art.

Another approach enabled by this invention, is the computational screening of small molecule databases for chemical entities or compounds that can bind in whole, or in part, to a caspase-7 binding pocket. In this screening, the quality of fit of such entities to the binding site may be judged either by shape complementarity or by estimated interaction energy [E. C. Meng et al., J. Comp. Chem., 13, pp. 505-524 (1992)].

Thus, enabled by this invention are compounds that inhibit caspase-7 by associating directly with the caspase-7 active site. Preferably, such compounds have a strain energy of 10 kcal/mol or less. More preferably, these compounds contain fewer than three secondary amide bonds. Even more preferably, these compounds have a molecular weight of less than 1000.

Those of skill in the art will realize that association of natural ligands or substrates with the binding pockets of their corresponding receptors or enzymes is the basis of many biological mechanisms of action. Similarly, many drugs exert their biological effects through association with the binding pockets of receptors and enzymes. Such associations may occur with all or any parts of the binding pockets. An understanding of such associations will help lead to the design of drugs having more favorable associations with their target receptor or enzyme, and thus, improved biological effects. Therefore, this information is valuable in designing potential ligands or inhibitors of receptors or enzymes, such as inhibitors of caspase-7.

The term “associating with” refers to a condition of proximity between chemical entities or compounds, or portions thereof. The association may be non-covalent—wherein the juxtaposition is energetically favored by hydrogen bonding or van der Waals or electrostatic interactions—or it may be covalent.

Another particularly useful drug design technique enabled by this invention is iterative drug design. Iterative drug design is a method for optimizing associations between a protein and a compound by determining and evaluating the three-dimensional structures of successive sets of protein/compound complexes.

In iterative drug design, crystals of a series of protein or protein complexes are obtained and then the three-dimensional structures of each crystal is solved. Such an approach provides insight into the association between the proteins and compounds of each complex. This is accomplished by selecting compounds with inhibitory activity, obtaining crystals of this new protein/compound complex, solving the three-dimensional structure of the complex, and comparing the associations between the new protein/compound complex and previously solved protein/compound complexes. By observing how changes in the compound affected the protein/compound associations, these associations may be optimized.

In some cases, iterative drug design is carried out by forming successive protein-compound complexes and then crystallizing each new complex. Alternatively, a pre-formed protein crystal is soaked in the presence of an inhibitor, thereby forming a protein/compound complex and obviating the need to crystallize each individual protein/compound. complex. Advantageously, the caspase-7/inhibitor crystals provided by this invention may be soaked in the presence of a compound or compounds, to provide other crystal complexes.

As used herein, the term “soaked” refers to a process in which the crystal is transferred to a solution containing the compound of interest.

In another embodiment of this invention is provided a method for preparing a composition comprising a caspase-7 comprising the steps described in Examples 1 and 2. Preferably, the composition comprises a caspase-7 in complex with Ac-DEVD-CHO.

Once the structure coordinates of a protein crystal have been determined they are useful in solving the structures of other crystals. Thus, the structure coordinates set forth in FIG. 7 can also be used to aid in obtaining structural information about another crystallized molecule or molecular complex. This may be achieved by any of a number of well-known techniques, including molecular replacement.

A machine-readable data storage medium comprising a data storage material encoded with a first set of machine readable data which, when combined with a second set of machine readable data using a machine programmed with instructions for using said first set of data and said second set of data, can determine at least a portion of the structure coordinates corresponding to the second set of machine readable data, wherein: said first set of data and said second set of data comprises a Fourier transform of at least a portion of the structural coordinates for caspase-7 according to FIG. 7; said second set of data comprises an X-ray diffraction pattern of a molecule or molecular complex of unknown structure.

The structure coordinates set forth in FIG. 7 can also be used for determining at least a portion of the three-dimensional structure of molecules or molecular complexes which contain at least some structurally similar features to caspase-7. In particular, structural information about another crystallized molecule or molecular complex may be obtained. This may be achieved by any of a number of well-known techniques, including molecular replacement.

Therefore, in another embodiment this invention provides a method of utilizing molecular replacement to obtain structural information about a crystallized molecule or molecular complex whose structure is unknown comprising the steps of:

a) generating an X-ray diffraction pattern from said crystallized molecule or molecular complex; and

b) applying at least a portion of the structure coordinates set forth in FIG. 7 to the X-ray diffraction pattern to generate a three-dimensional electron density map of the molecule or molecular complex whose structure is unknown.

Preferably, the crystallized molecule or molecular complex comprises a caspase-7. More preferably, the crystallized molecule or molecular complex is obtained by soaking a crystal of this invention in a solution.

By using molecular replacement, all or part of the structure coordinates of the caspase-7 complex provided by this invention (and set forth in FIG. 7) can be used to determine the structure of a crystallized molecule or molecular complex whose structure is unknown more quickly and efficiently than attempting to determine such information ab initio.

Molecular replacement provides an accurate estimation of the phases for an unknown structure. Phases are a factor in equations used to solve crystal structures that can not be determined directly. Obtaining accurate values for the phases, by methods other than molecular replacement, is a time-consuming process that involves iterative cycles of approximations and refinements and greatly hinders the solution of crystal structures. However, when the crystal structure of a protein containing at least a homologous portion has been solved, the phases from the known structure provide a satisfactory estimate of the phases for the unknown structure.

Thus, this method involves generating a preliminary model of a molecule or molecular complex whose structure coordinates are unknown, by orienting and positioning the relevant portion of the caspse-7 complex according to FIG. 7 within the unit cell of the crystal of the unknown molecule or molecular complex so as best to account for the observed X-ray diffraction pattern of the crystal of the molecule or molecular complex whose structure is unknown. Phases can then be calculated from this model and combined with the observed X-ray diffraction pattern amplitudes to generate an electron density map of the structure whose coordinates are unknown. This, in turn, can be subjected to any well-known model building and structure refinement techniques to provide a final, accurate structure of the unknown crystallized molecule or molecular complex [E. Lattman, in Meth. Enzymol., 115, pp. 55-77 (1985); M. G. Rossmann, ed., Int. Sci. Rev. Ser., No. 13, Gordon & Breach, New York (1972)].

The structure of any portion of any crystallized molecule or molecular complex that is sufficiently homologous to any portion of the caspase-7 can be solved by this method.

In a preferred embodiment, the method of molecular replacement is utilized to obtain structural information about a molecule or molecular complex, wherein the complex comprises a caspase-7. Preferably the caspase-7 is the caspase-7 described herein, in complex with the inhibitor Ac-DEVD-CHO.

The structure coordinates of caspase-7 as provided by this invention are particularly useful in solving the structure of other crystal forms of caspase-7, or complexes thereof.

The structure coordinates are also particularly useful to solve the structure of crystals of caspase-7 complexes, particularly caspase-7, co-complexed with a variety of chemical entities. This approach enables the determination of the optimal sites for interaction between chemical entities, including interaction of candidate caspase-7 inhibitors with caspase-7. For example, high resolution X-ray diffraction data collected from crystals exposed to different types of solvent allows the determination of where each type of solvent molecule resides. Small molecules that bind tightly to those sites can then be designed and synthesized and tested for their caspase-7 inhibition activity.

All of the complexes referred to above may be studied using well-known X-ray diffraction techniques and may be refined versus 1.5-3 Å resolution X-ray data to an R value of about 0.20 or less using computer software, such as X-PLOR [Yale University, ©1992, distributed by Molecular Simulations, Inc.; see, e.g., Blundell & Johnson, supra; Meth. Enzymol., vol. 114 & 115, H. W. Wyckoff et al., eds., Academic Press (1985)]. This information may thus be used to optimize known caspase inhibitors, and more importantly, to design new caspase inhibitors.

In order that this invention be more fully understood, the following examples are set forth. These examples are for the illustrative purposes only and are not to be construed as limiting the scope of this invention in any way.

### EXAMPLE 1

### Expression and Purification of Caspase-1, Caspase-3, Caspase-7, and Caspase-8 for Crystallization

Recombinant human Caspase-1 was expressed in Escherichia coli as an insoluble p32 protein spanning residues 120-404 of the p45 precursor [N. A. Thornberry et al., Nature, 356, pp. 768-774 (1992)]. The insoluble p32 protein was solubilized and purified under chaotropic conditions, then refolded and autoprocessed in vitro producing the p20 and p10 active subunits. Details, including previous X-ray crystallographic analyses have been described elsewhere [K. P. Wilson et al., Nature, 370, pp. 270-275 (1994); N. Margolin et al., J. Biol. Chem., 272, pp. 7223-7228 (1997)]. Caspase-3 (residues 29-277) and Caspase-7 (residues 1-303) containing an N-terminal (His)₆ affinity tag and thrombin cleavable site were expressed in Escherichia coli [Y. Gu et al., J. Biol. Chem., 271, pp. 10816-10820 (1996); J. A. Lippke et al., J. Biol. Chem. 271, pp. 1825-1828 (1996)]. Both caspases were soluble and active as their p20/p10 subunits, yielding 0.05 and 1 mg/gm cells of caspase-3 and caspase-7, respectively. For purification of either protein, cell paste was resuspended in 10 volumes of 50 mM HEPES buffer containing 10% (v/v) glycerol, 300 mM NaCl, 5 mM β-mercaptoethanol (β-ME), 0.05% (w/v) β-OG, 25 mM imidazole, 0.1 mM PMSF, pH 8.0 at 4° C. Following mechanical disruption of the cells, the soluble fraction was harvested by centrifugation at 30,000×g for 30 min at 4° C. The supernatant was incubated batchwise overnight with 1 ml Ni-affinity resin (Qiagen) per 5-10 mg of expected caspase-3 or caspase-7. The resin was washed with 50 column volumes of the extraction buffer, followed by 50 column volumes of the extraction buffer adjusted to pH 7.0. caspase-3 and caspase-7 were eluted with 50 and 300 mM imidazole, respectively, in 50 mM HEPES, pH 7.0 containing 10% (v/v) glycerol, 100 mM NaCl, 5 mM β-ME.

Recombinant caspase-8 (residues 233-479) was expressed as an N-terminal His-tagged fusion in High-five insect cells using a baculovirus expression vector system by conventional methods [W. Chen et al., Protein Expression and Purification, 9, pp. 69-75 (1997)]. Crystallization of caspase-8/Ac-DEVD-CHO was enhanced by three Lys→Arg point mutations (at residues 246, 250 and 253), and removal of Pro370 and Val371, which appeared absent from other caspases in sequence alignments. Caspase-8 was processed intracellularly and secreted into the media during expression. The cell culture media was centrifuged at 1,600×g to remove cells, and the supernatant (˜pH 6.4) adjusted to 50% (w/v) ammonium sulfate and stirred gently for 60 min on ice. After centrifugation at 54,000×g for 45 min at 4° C., the supernatant was decanted, 0.2 μm filtered and the ammonium sulfate concentration increased to 85% (w/v) and stirred for another 60 min on ice. Centrifugation at 54,000×g for 45 min at 4° C. resulted in the precipitation of caspase-8. Pellets were resuspended in 2% of the starting volume of the media using 100 mM HEPES, pH 8.0, containing 100 mM NaCl, 10% glycerol (v/v) and 5 mM 1-ME. The solution was incubated with 1 ml Talon affinity resin (Clontech) per 5-10 mg expected caspase-8 and gently mixed overnight at 40C. The resin washed with 150 column volumes of 20 mM HEPES, pH 7.0, containing 100 mM NaCl, 10% glycerol (v/v) and 5 mM β-ME. A second wash of 50 column volumes was performed using the same buffer with 25 mM imidazole. Caspase-8 was eluted with wash buffer adjusted to 350 mM imidazole.

Characterization of Triple Mutant Caspase-8

Wild-type and mutant caspase-8 enzymes were characterized using the fluorogenic substrate Ac-DEVD-AMC (Alexis Biochemicals, San Diego, Calif.). The concentrations of active enzyme were determined by active site titration with Ac-DEVD-CHO (Peptides International, Louisville, Ky.). All assays were performed in 100 mM HEPES, pH 8, containing 100 mM NaCl, 5 mM DTT and 0.1% (w/v) CHAPS at 37° C. using a 96-well Fmax plate reader (Molecular Devices, Sunnyvale, Calif.). The Ac-DEVD-AMC substrate concentration was varied between 2 and 100 μM and the reaction was initiated by addition of 2 nM enzyme. Enzyme kinetic data were analyzed by nonlinear regression in the program EZ-Fit (Perrella Scientific, Amhurst, N.H.).

### EXAMPLE 2

### Crystallization of Caspase-1, Caspase-3, Caspase-7, and Caspase-8 in Complex with Ac-DEVD-CHO

Details of protein purification and crystallization of the caspase-1/Ac-DEVD-CHO complex has been reported [N. Margolin et al., J. Biol. Chem., 272, pp. 7223-7228 (1997)]. Metal affinity purified caspase-3, caspase-7 or caspase-8 were inhibited by addition of a two-fold molar excess of Ac-DEVD-CHO (Peptides International). The N-terminal (His) 6 tag was then removed from caspase-3, caspase-7 or caspase-8 by thrombin cleavage (20 units of thrombin/mg caspase) at 37° C. for 60 min. Thrombin was removed by a 5 min incubation with 100 μl of benzamidine sepharose. The free (His)₆ tag and aggregated caspase were removed by size-exclusion chromatography using a column (60×1.5 cm) packed with Superdex-75 resin (Pharmacia). The column was equilibrated at 4° C. in 20 mM HEPES, pH 7.0, containing 10% glycerol (v/v), 100 mM NaCl, 5 mM β-ME at a flow rate of 1 ml/min. Light-scattering (PD-2000, Precision Detectors, Franklin, Mass.) analyses during size-exclusion chromatography identified aggregated protein, which was excluded from the pooled fractions. Caspase-1/Ac-DEVD-CHO was purified on the same size-exclusion column except 50 mM citrate, pH 6.5, containing 2 mM DTT buffer was used. After size-exclusion, equimolar Ac-DEVD-CHO was added prior to concentration of the caspase/Ac-DEVD-CHO co-complexes for crystallization. All protein samples were stored at −70° C.

Crystals of inhibited caspase-3 were grown by vapor diffusion and macro-seeding. Thousands of micro-crystals were initially obtained in 12 h when protein (4.6 mg/ml in 20 mM Na HEPES, 2.0 mM DTT, 0.1 M NaCl, 10% glycerol, pH 7.0) was mixed with reservoir (0.2 M ammonium acetate, 0.1 M Na citrate, 30% w/v PEG 4000) at a 3 μl:2 μl protein solution to reservoir ratio and allowed to stand at room temperature. A crystallization droplet setup in the same way but with 2% (v/v) methyl-pyrrolidinone (MeP) added to the reservoir produced no crystals. A micro-crystal was then transferred to this second drop and this seed crystal grew over 10 days to a size of 0.4 mm×0.3 mm×0.25 mm.

The autoproteolyzed active form caspase-7 was inhibited by titrating Ac-DEVD-CHO into the protein sample. The complex was further purified by size-exclusion chromatography. Pooled fractions were concentrated to about 5 mg/ml for crystallization. Slow vapor diffusion was used to obtain X-ray quality crystals of the caspase-7/Ac-DEVD-CHO complex over a few weeks at 4° C.

Caspase-8 was titrated with Ac-DEVD-CHO (12.0 mg/ml, 20 mM HEPES pH 7.0, 0.1 M NaCl, 5.0 mM β-ME) and was subsequently added to a reservoir solution (0.1 M potassium phosphate, pH 6.0, 5% t-butanol, 40% (w/v) ammonium sulfate) at a 2 μl:2 μl ratio and suspended over 1.0 ml of reservoir at room temperature. A single crystal was harvested within two weeks and has dimensions of 0.40×0.20×0.10 mm. The same crystal was flash cooled to 170° K in N₂ gas stream prior to data collection.

### EXAMPLE 3

### Crystal Structure Determination

Crystals of caspase-1-Ac-DEVD-CHO and caspase-3-Ac-DEVD-CHO were mounted in glass capillaries for X-ray data collection at −7° C. and −4° C. respectively. X-ray data of both caspase-1/Ac-DEVD-CHO and caspase-3 complexes were collected on a Raxis IIC image plate equipped with Rigaku rotating anode generator and processed using software provided by the manufacture (Molecular Structures Corp., Woodlands, Tex.). R-merge for the data was 6.1% at 2.2 Å resolution. Analysis of the unit cell dimensions suggested that each asymmetric unit contained two caspse-3 heterodimers. A polyalanine model of a single caspase-1 heterodimer was used to obtain a successful rotation and translation function solution for a caspase-3 heterodimer using the program AMoRe [J. Navaza, Acta Crystallography, A50, pp. 157-163 (1994)]. The first solution was then held fixed while a second polyalanine model was tried in the rotation and translation function. Combining the two solutions produced a polyalanine model for the caspase-3 dimer of heterodimers with an R-factor of 45.1% for all observed reflections between 8 and 2.8 Å resolution, and an R-free of 47.5% for 10% of the reflections set aside at the start of the refinement. The resolution of the maps and model was gradually increased to 2.2 Å resolution by cycles of model building, positional refinement and thermal factor refinement, interspersed with torsional dynamics runs. All model refinement was carried out using the XPLOR suite of programs [A. Brunger, “X-PLOR, A system for X-ray crystallography and NMR” New Haven, Yale University Press (1996)].

Crystals of the caspase-7-Ac-DEVD-CHO complex were transferred to cryoprotectant and flash-cooled to 100K in N₂ gas stream prior to data collection. The diffraction images were recorded on CCD 2×2K detector at Brookhaven National Laboratories (BNL), Brookhaven, N.Y. The data were processed using DENZO and SCALPACK software [Z. Otwinowski & W. Minor, Methods In Enzymology (Macromolecular Crystallography, Part A), 276, pp. 307-326 (1997)]. The crystals have unit cell dimensions of 88.2 Å, 88.2 Å, 186.2 Å, α=90.0°, β=90.0°, γ=120.0° and belong to space group P3221. Assuming that there is one tetramer in the asymmetric unit, the calculated Matthew's specific volume was 2.6 Å³/d. The structure was solved by molecular replacement methods using a truncated caspase-3 tetramer molecule as the searching template. The initial R-factor and correlation coefficient factors are 44% and 65%, respectively. A polyalanine model from the solution was first refined against data between 8.0 Å to 3.0 Å. The side chains of individual amino acids were modeled into the electron density map according to the protein sequence. The model was refined using XPLOR and manually corrected using QUANTA (QUANTA97, Molecular Simulations, Inc.). The final model has a full tetramer assembly of 654 residues, two sulfate anions and 374 water molecules.

A single crystal of caspase-8-Ac-DEVD-CHO complex was flash cooled in N₂ cold gas stream prior to the synchrotron data collection at BNL. Data was recorded on a 2×2K CCD image plate mounted on X25 beam line. The space group of the crystal was determined as C2221 with unit cell of 62.12 Å, 344.33 Å, 190.99 Å, α=90.0, β=90.0, γ=90.0. The Matthew's specific volume calculation suggested that there are three independent tetrameric molecules in the asymmetric unit giving a calculated solvent content of 54% [B. W. Matthews, Journal of Molecular Biology, 33, pp. 491-497 (1968)]. The crystal diffracts extremely anisotropically along the a*, b*, and c* axes to 2.35 Å, 2.80 Å and 2.65 Å respectively. The data set was processed using DENZO and SCALPACK software to a resolution of 2.65 Å. The structure solution was obtained by using AMoRe and by using a truncated caspase-3 tetramer molecule as searching template. Rigid-body and positional refinement of the polyalanine model was initially done using XPLOR. The side chains, insertions and deletions of the molecule were modeled into the electron density map manually using QUANTA programs. The final model contains 1454 amino acids, 316 solvent molecules and six Ac-DEVD-CHO compounds covalently attached to the active site Cysteine residue.

The coordinates of the caspase-7-Ac-DEVD-CHO structure have been deposited with the Protein Data Bank under the accession code 1F1J.

### EXAMPLE 4

### Crystallography

The model quality of all four structures was assessed using PROCHECK and the crystallographic statistics are given in Table 1 [R. A. Laskowski, Journal of Applied Crystallography, 26, pp. 283-291 (1993)].

TABLE 1
Crystallographic data and refinement statistics

| | Csp1 | Csp3 | Csp7 | Csp8 |
|---|---|---|---|---|
| X-ray experiments | | | | |
| Temperature (K) | 298 | 270 | 100 | 100 |
| X-ray radiation | rotating anode | rotating anode | BNSL | BNSL |
| Wavelength (Å) | 1.5418 | 1.5418 | 1.10 | 1.09 |
| Space group | P4₃2₁2 | P2₁2₁2₁ | P3₂21 | C222₁ |
| a(Å) | 62.45 | 89.46 | 88.18 | 62.12 |
| b(Å) | 62.45 | 97.33 | 88.18 | 344.33 |
| c(Å) | 162.36 | 70.09 | 186.23 | 190.99 |
| α(°) | 90.0 | 90.0 | 90.0 | 90.0 |
| β(°) | 90.0 | 90.0 | 90.0 | 90.0 |
| γ(°) | 90.0 | 90.0 | 120.0 | 90.0 |
| No of p20/p10 in asym. Unit | 1 | 2 | 2 | 6 |
| Recording apparatus | Raxis IIC | Raxis IIC | CCD 2X2K | CCD 2X2K |
| Resolution (Å) | 2.5 | 2.2 | 2.35 | 2.65 |
| Total observation | 48936 | 63425 | 139781 | 189632 |
| Unique reflection | 13468 | 12532 | 34359 | 53183 |
| Completeness (%) | 93 | 92 | 96.6 | 88.2 |
| R_{sym}* (%) | 6.5 | 7.8 | 8.3 | 5.5 |
| Structure solution | | | | |
| Methods | ref 16. | M.R. | M.R. | M.R. |
| Search model | ref 16. | polyala of Csp1 | polyala of Csp3 | polyala of Csp3 |
| Resolution range (Å) | ref 16. | 8.0-2.8 | 16.0-4.0 | 16.0-4.0 |
| Software | ref 16. | Amore | Amore | Amore |
| R-factor (%) | ref 16. | 45.1 | 41.0 | 34.7 |
| Correlation coefficient Factor | ref 16. | 54.6 | 61.0 | 72.5 |
| Refinement | | | | |
| Resolution range(Å) | 7.5-2.4 | 8.0-2.2 | 7.5-2.35 | 20.0-2.65 |
| Sigma cut off (σ) | 2.5 | 3.0 | 2.5 | 2.5 |
| Programs | XPLOR | XPLOR | XPLOR | XPLOR |
| No of reflections | 14658 | 24890 | 33303 | 48348 |
| R-factor^{#} (%) | 23.1 | 18.9 | 18.5 | 21.9 |
| Free R-factor^{#} (%) | 28.2 | 24.7 | 26.3 | 28.6 |
| Stereochemical parameters | | | | |
| R.m.s. bond distance (Å) | 0.011 | 0.009 | 0.009 | 0.006 |
| R.m.s. bond angle (°) | 2.3 | 1.9 | 1.8 | 1.7 |
| R.m.s. dihedral angle (°) | 24.1 | 23.6 | 23.4 | 23.4 |
| Non-glycine residues | 90.0 | 89.3 | 89.0 | 86.3 |
| in the regions of | | | | |
| Most favored | | | | |
| Additional allowed | 9.5 | 9.0 | 10.5 | 13.7 |
| Generously allowed | 0.5 | 0.7 | 0.5 | 0.0 |
| Disallowed | 0 | 0 | 0 | 0.0 |

*R_{sym} = Σ|Iᵢ − <I>|/ΣIᵢ Where Iᵢ and <I> are the intensities for the ith observation and mean of the reflection, respectively
^{#}R-factor = Σ|F_{obs} − F_{cal}|/Σ|F_{obs}| Where F_{obs} and F_{cal} are observed and calculated model structure factors.

Comparison of Caspase-7/Ac-DEVD-CHO Complex to Caspase-3/Ac-DEVD-CHO Complex

The caspase-7-Ac-DEVD-CHO complex is similar to the caspase-3-Ac-DEVD-CHO complex. The refined model of caspase-7 contains a complete catalytic unit comprising two p20-p10 heterodimers. The p20 and p10 polypeptide chains are composed of residues 57-196 and 212-302, respectively. The two interdigitating heterodimers associate to form a tetramer of globular shape. A ribbon stereo diagram of the caspase-7 complex is illustrated in FIG. 1. As expected, the overall fold of caspase-7 is very similar to that of caspase-1, caspase-3, and caspase-8. All four subunits contribute to a total of 12 strands comprising the central β sheet. This forms the core of the enzyme, which is flanked by ten a helixes approximately parallel to the β strands. In particular, the β-sheet structure of the p20 domain, which contains the catalytic His285 and Cys186 residues, has a crossing-over −1×, +2×, +1× linking topology according to Richardson's definition [J. S. Richardson, Adv. Protein Chem., 34, pp. 167-338 (1981)]. This motif has been widely observed in serine hydrolases, most notably the α/β hydrolase superfamily [D. L. Ollis et al., Protein Eng., 5, pp. 599-611 (1992)].

Among the three subfamilies of caspases, caspase-7 belongs to the second group that includes caspase-2 and caspase-3. Aside from the differences that exist in the propeptide and linker regions, caspase-7 is structurally and functionally very similar to caspase-3. In fact, caspase-7 and caspase-3 can be superimposed without any amino acid deletions or insertions along the polypeptide chains (FIG. 2a) with a root mean square (RMS) deviation of 0.37 Å for backbone Ca atoms. Sequence alignment and Ca atom superpositioning of caspase-1, caspase-3, caspase-7 and caspase-8 spanning all three caspase subfamilies (FIGS. 2a and 3) demonstrated that the main differences between their folds are in three regions. These differences occur in the two loops on the prime side and a third loop proximal to the S4 binding site (FIG. 2a).

Similar to caspase-3, caspase-7 also has a single-residue deletion within the strand C290-M294 (FIG. 3), which pairs with its symmetry-related equivalent forming the tetrameric assembly of the enzyme. In caspase-1 and other caspases of group 1, there is an arginine residue (R391 in caspase-1) which causes a bulge in the β strand and consequently induces a rotation of one heterodimer relative to the other. In caspase-3 [P. R. E. Mittl et al., J. Biol. Chem., 272, pp. 6539-6547 (1997)] and caspase-7, the two dimeric subunits are significantly less twisted. The combination of this single deletion and greater hydrophobic nature of this strand in caspase-3 and caspase-7 has not only changed the quaternary structures of these enzymes but also reshaped the central cavity formed at the interface of the two heterodimers. The central cavity of the caspase-7 tetramer was occupied by 24 water molecules. As this central cavity is adjacent to the prime side of the substrate binding site, it may not effect directly substrate binding to caspase-3 and caspase-7. However, the variation in the central cavity among caspases might be an important determinant of macromolecular substrate recognition in the apoptosis cascade.

Conserved Fold And Topology

Like caspase-1 and caspase-3, the mature forms of caspase-7 and caspase-8 are tetramers comprised of two p20/p10 heterodimers. The heterodimers are both structurally and functionally associated to appear as a single domain. For all four caspases, the protein core is a six-stranded (β-sheet surrounded by five or six α-helices which lie roughly parallel to the β-strands. Weak electron density for the N-terminal helix was observed for Csp1 [K. P. Wilson & D. J. Livingston, Nature, 370, pp. 270-275 (1994)], but little or no density could be observed for this helix in caspase-3, caspase-7, and caspase-8. Both p10 and p20 subunits contribute to the ligand binding site for all four caspase structures. The main differences between the folds for caspases 1, 3, 7, and 8 occur in two loop regions: one on the prime side and the other proximal to the S4 binding site (FIG. 2A).

On the prime side, caspase-8 differs from the others by the presence of a helix-turn-helix insertion ranging from residues 245-253 (FIG. 2C). This agrees with the recently reported caspase-8 structures expressed in Escherichia coli that also possess some helical content to this insertion loop [H. Blanchard et al., Structure (London), 7, pp. 1125-1133 (1999); W. Watt et al., Structure (London), 7, pp. 1135-1143 (1999)]. An analysis of a sequence alignment derived from superposition of the four caspase structures (FIG. 3), suggests this insertion to be unique to caspase-8. Three of the lysines in this region (Lys246, Lys250, Lys253) were mutated to arginine in order to obtain diffraction quality crystals. A comparison of wild-type and the mutant form of caspase-8 used to determine the crystal structure showed that both enzymes appear to be indistinguishable, yielding a kcat/Km of 1×10⁵ M1-s¹-¹ for Ac-DEVD-aminoemethylcoumarin (Ac-DEVD-AMC) hydrolysis. Two of these mutated arginines are stabilized by a salt bridge with nearby glutamic residues (Arg250 with Glu249, Arg253 with Glu330, Arg246 with a glutamic acid from a symmetry-related neighbor. None of these mutated residues is oriented in the prime side region nor are they in close proximity to the tetrapeptide inhibitor. Leu254, from this helix-turn-helix insertion, appears to point inward towards the prime side region. The spatial position of the helix-turn-helix insertion for caspase-8 most closely resembles a short insertion loop of caspase-1 (FIG. 2C: residues 249-254) which is conserved among the members of the caspase-1 family.

The differences in size of the S4 loop among the four caspases in this study are striking. These loops involve the following residues: caspase-1 (378-386), caspase-3 (244-262), caspase-7 (270-288), caspase-8 (451-463). FIG. 2B illustrates that this loop is largest for caspase-3 and caspase-7. Despite the fact that this loop is identical in length for these two closely related caspases, they are only 63% identical in composition. The same loop is shorter for caspase-8 and shorter still for caspase-1. In each case there is an interaction between the P4 aspartic acid of the Ac-DEVD-CHO inhibitor and a residue in this loop.

Binding of Ac-DEVD-CHO to Caspases from all Three Families

The four caspases included in this application span three different functional and phylogenetic groups of caspases and each exhibit subtle differences in the way they bind Ac-DEVD-CHO. Although there are differences in some of the protein amino acids from each caspase contributing to binding Ac-DEVD-CHO, the overall conformation of the inhibitor is highly conserved. The peptide inhibitor has adopted an extended conformation in all four complexes. Besides the fact that the catalytic dyad and two residues anchoring the aspartate residue in the P1 position are from the p20 domain, most of the intimate contacts are between the tetrapeptide and the groups from the p10 subunit. Although there are differences in some of the amino acid residues from each caspase that contribute to binding Ac-DEVD-CHO, the overall conformation of the inhibitor is highly conserved. A schematic comparing Ac-DEVD-CHO binding to caspase-1, caspase-3, caspase-7, and caspase-8 is shown in FIG. 4, whereas FIG. 5 shows the surface features of the respective binding sites.

Comparison of the Binding of Caspase-1, Caspase-2, Caspase-3 and Caspase-4 to the Tetrapeptide P1-P4 Residues

A schematic comparing Ac-DEVD-CHO binding to caspase-1, caspase-3, caspase-7, and caspase-8 is shown in FIG. 4. FIG. 5 shows the surface features of the respective binding sites.

At the P1 site, all four of the complexes show that the inhibitor is covalently bound to the nucleophilic cysteine in the active site (FIG. 4) although binding to the classic oxyanion hole was not observed. In each case, the thiohemiacetal oxygen resulting from nucleophilic thiol attack on the aldehyde carbonyl forms a hydrogen bond with the adjacent histidine that comprises the enzymes's cysteine-histidine dyad. In addition, the thiohemiacetal oxygen in all four complexes makes hydrogen bonds of varying strength to the nucleophilic cysteine backbone nitrogen and conserved glycine backbone nitrogen. Previous work has shown that some irreversible inhibitors exhibit classic oxyanion hole binding to caspase-1 and caspase-3 [N. P. C. Walker et al., Cell, 78, pp. 343-352 (1994); P. R. E. Mittl et al., J. Biol. Chem., 272, pp. 6539-6547 (1997)). The other interactions at P1 are also strikingly conserved among the four structures. In every case, there are charge-charge interactions between the P1 aspartate sidechain and two arginine residues, as well as a hydrogen bond with a conserved glutamine. FIG. 5 shows the conserved nature of this overwhelmingly electropositive site and the buried nature of the P1 aspartate. Additionally, each complex retains a hydrogen bond between the P1 backbone nitrogen and the backbone carbonyl of a conserved serine.

For the P2 valine of Ac-DEVD-CHO the overall nature of the hydrophobic interaction is conserved, but there are subtle differences in the χ¹ value for the valine sidechain depending on the residues comprising the S2 pocket. Interestingly, caspase-3 and caspase-7 possess an extra residue (phenylalanine) which contributes to the S2 site and is also part of the S4 loop. Caspase-8 also possesses an aromatic residue (Tyr365) at roughly the same spatial position as Phe256 of caspase-3 and Phe282 of caspase-7, but this tyrosine residue is part of an extended strand near the C-terminus of the p20 subunit. Residues corresponding to Tyr365 of caspase-8 are Pro290 (caspase-1), Leu168 (caspase-3, and Leu191 caspase-7). None of these contribute directly to the S2 site in these complexes with Ac-DEVD-CHO.

The P3 glutamic acid of the tetrapeptide inhibitor, makes at least one charge-charge interaction with a conserved arginine in all four caspase complexes (FIG. 4). Additionally, the P3 backbone nitrogen and carbonyl oxygen of the P3 glutamic acid make strong hydrogen bonds with the respective backbone carbonyl oxygen and nitrogen of the same arginine involved in the surface charge interaction. Caspase-8 also has an additional charge-charge interaction from a second arginine (Arg258) which follows the helix-turn-helix insertion loop. This is in agreement with recent findings in which a similar peptide inhibitor is complexed with caspase-8 [H. Blanchard et al., Structure (London), 7, pp. 1125-1133 (1999)]. The sequence alignment presented in FIG. 3 suggests that caspase-6, caspase-9, caspase-10 and caspase-14 would also be capable of providing an extra basic amino acid at this position. Analysis of FIG. 5 shows that the P3 glutamic acid of the Ac-DEVD-CHO inhibitor is oriented towards this second (i.e. Arg258) arginine on the protein surface of caspase-8. A crystallographic water is observed binding to the conserved arginine for caspase-3, caspase-7, and caspase-8.

The major differences for the binding of Ac-DEVD-CHO to caspase-1, caspase-3, caspase-7, and caspase-8 occur at the P4 aspartic acid and the acetylated amino terminus of the tetrapeptide. All of the caspases included in this study involve either direct or indirect binding by the S4 loop. FIG. 4 shows that caspase-1 interacts with the P4 aspartic acid sidechain directly through an interaction with Arg383. Caspase-3 interacts via the backbone nitrogen of Phe250 and the sidechain of Asn208. In this case, there is also an interaction of the P4 aspartic acid with Trp214 through a water mediated hydrogen bond. Additionally, the P4 backbone nitrogen and carbonyl oxygen forms hydrogen bonds to two different water molecules both of which form hydrogen bonds to the carbonyl of Phe250. Caspase-7 binds the P4 aspartic acid through both backbone and sidechain involvement of Gln276. The P4 backbone nitrogen forms an additional hydrogen bond with the backbone carbonyl of Gln276 while the P4 backbone carbonyl oxygen forms a hydrogen bond to the same crystallographic water molecule involved in another hydrogen bond with the P2 backbone nitrogen. It is interesting to note that although the caspase-3 and caspase-7 sequences share 57% identity and 67% similarity (FIG. 3) there are significant differences in and around the S4 binding region. The replacement of caspase-3 residues (Asn208, Ser209, Asp211, Phe25O, Phe252, Thr255, and Ala258 by their respective caspase-7 residues (Ser234, Pro235, Arg237, Gln276, Asp278, His281, and Glu284) has changed the chemical content (FIG. 5) in the S4 binding region to be more hydrophilic.

Caspase-8 interacts directly with the P4 aspartate residue via the sidechain nitrogens of Asn414 and Trp420 that are not part of the S4 loop. However, the P4 aspartate carbonyl oxygen interacts with D455 via a water mediated hydrogen bond.

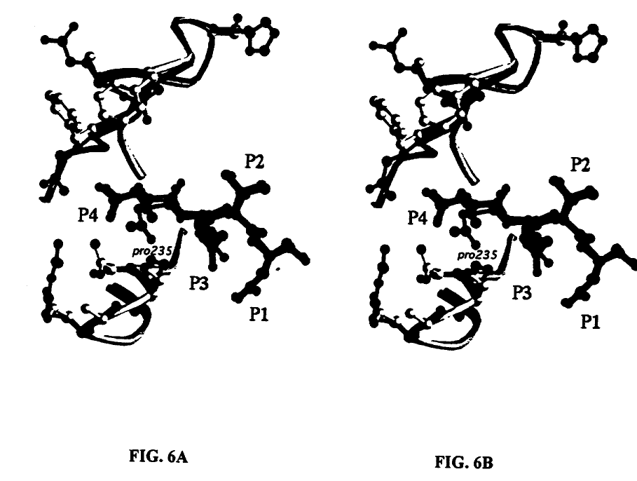

The N-acetyl P4 capping group also exhibits differences in hydrogen bonding and hydrophobic interactions. Caspase-1, caspase-7, and caspase-8 all make a hydrophobic interaction between the N-acetyl terminal methyl and a proline (FIG. 4). Caspase-3 lacks this hydrophobic interaction, but makes two hydrogen bonds with S209, one involving the S209 backbone nitrogen, the other with the S209 sidechain. Caspase-7 forms a water mediated hydrogen bond with both the backbone nitrogen and sidechain of Asp278. For caspase-8, the N-acetyl carbonyl oxygen forms a hydrogen bond to the sidechain of Asn414. No hydrogen bond is observed between caspase-1 and the N-acetyl group. One final observation regarding differences in binding the N-acetyl group occurs between caspase-3 and the other three caspases included in this study. As mentioned above, there are two strong hydrogen bonds between the N-acetyl group and S209 for caspase-3. In caspase-1, caspase-7, and caspase-8, (and caspase-9) however, the residue corresponding to Ser209 is a proline; any hydrogen bonding interactions of the N-acetyl group involve different residues. Due to the inability of the proline ring to form hydrogen bonds, the P4 N-acetyl group is translated approximately 2.5 Å. FIG. 6 illustrates this shift between caspase-3 and caspase-7. These are members of the same caspase subfamily.

The differences in hydrogen bonding of the P4 and N-acetyl groups with the associated caspase residues results in a variable width of the S4 site with caspase-8 having the widest S4 site followed by caspase-1 (FIG. 5); caspase-3 and caspase-7 have a narrower S4 site. For caspase-3 and caspase-7 the reduced width in the S4 site can be directly linked to the greater extent of the hydrogen bond network between this portion of the tetrapeptide and the S4 site which serves to “pull” the walls of the S4 pocket towards the P4 group. On this point, the electrostatic potential surfaces illustrated in FIG. 5 are informative. The surface potential of caspase-7 is unique because it is the only caspase of the four studied which has an unpaired basic residue (Arg237) near the P4 Asp. Caspase-1 and caspase-8 also have basic residues that contribute to the electropositive portion of the surface potential in this region, but they also have counterbalancing negative charges near by. Caspase-3 has a neutral S4 region. These observations suggest that in terms of structure and associated surface potential, there should be no absolute requirement for a negatively charged group at P4.

Structural Insights and Binding Specificity

Based on positional scanning of a combinatorial substrate library, Thornberry et al. were able to determine the optimal tetrapeptide substrate sequences for ten human caspases (N. A. Thornberry, J. Biol. Chem., 272, pp. 17907-17911 (1997)]. In this study, a cleavable Asp-AMC was held constant at P1 and the amino acids at P2 through P4 were varied. They found that group I caspases (caspase-1, caspase-4 and caspase-5) loosely preferred the sequence WEHD, while group II caspases (caspase-2, caspase-3, and caspase-7) strongly preferred the motif DEXD. Group III caspases (caspase-6, caspase-8, caspase-9, and caspase-10) preferred the motif (L/V)EXD. This information was subsequently used to design and investigate tetrapeptide aldehyde inhibitors [M. Garcia-Calvo et al., J. Biol. Chem., 273, pp. 32608-32613 (1998)]. Some traditional reversible inhibitors and irreversible inhibitors such as Z-VAD-FMK were also included in the study.

Reported Ki values (nM) from reference sequences for Ac-DEVD-CHO against caspase-1, caspase-3, caspase-7 and caspase-8, respectively, are as follows: 18 nM, 0.23 nM, 1.6 nM, 0.92 nM [M. Garcia-Calvo et al., J. Biol. Chem., 273, pp. 32608-32613 (1998)]. Although this tetrapeptide inhibitor is most potent against group II and group III caspases, it is the only reported tetrapeptide aldehyde that significantly inhibits representatives from all three groups of caspases. The fact that all caspases considered here can accommodate a valine at P2 attests to the general tolerability for branched amino acids at this position. However, branched amino acids at P2 tend to produce less optimal substrates against caspase-1, caspase-4 and caspase-5 and are typically not present in the best inhibitors for group I caspases [N. A. Thornberry, J. Biol. Chem., 272, pp. 17907-17911 (1997)]. In general, large groups are tolerated as both substrates and inhibitors for all three caspase subfamilies and even though tetrapeptide substrates containing tryptophan, phenylalanine, and tyrosine at P2 are suboptimal, histidine-containing tetrapeptide substrates were generally good against group I and group III caspases [N. A. Thornberry, J. Biol. Chem., 272, pp. 17907-17911 (1997)]. Additionally, in a previous crystallographic study, a non-peptidic pyridone aldehyde inhibitor containing a large 6-benzyl substituent on the pyridone ring was found to place the phenyl ring of the benzyl group over the S2 site in caspase-1 [J. M. C. Golec, Bioorg. Med. Chem. Lett., 7, pp. 2181-2186 (1997)].

The optimal residue identified by the combinatorial tetrapeptide substrate library at the P3 position for all three groups of caspases is glutamic acid [N. A. Thornberry et al., J. Biol. Chem., 272, pp. 17907-17911 (1997)]. All four caspases in the present study make at least one charge-charge interaction with the P3 glutamic acid. The nature of this interaction involves an absolutely conserved Arg, however, this salt bridge is on the surface of the protein and is solvent exposed. In terms of substrate specificity, such an interaction might be optimal in terms of recognition and proper positioning of the peptide backbone, but would be expected to contribute minimally to the overall binding energetics (S. Dao-Pin, Ciba Found. Symp. (Protein Conform.) 161, pp. 52-62 (1991)]. This is illustrated by the inhibitor Ac-YVAD-CHO which shows good inhibitory potency against caspase-1 and modest inhibitory potency against group III caspases. Although the surface charge-charge interaction(s) would be lost, the P3 valine would be expected to still make the required backbone hydrogen bonds and a potential hydrophobic interaction with a either a proline (caspase-1, caspase-7, caspase-8) β-carbon of serine (caspase-3). The branched P3 valine would also serve to maintain the rigidity of the extended tetrapeptide inhibitor and could potentially improve cell potency due to one less formal charge on the molecule. A recent report of peptidomimetic caspase inhibitors shows reasonably potent inhibition of multiple caspases in a compound class that would be incapable of making a charge-charge interaction with the conserved S3 arginine [J. C. Wu & L. C. Fritz, Methods (Orlando, Fla.), 17, pp. 320-328 (1999)].

In terms of both substrate specificity and inhibitor selectivity, the P4 position offers the most variability. In general, both hydrophobic groups and the anionic aspartate are tolerated at the P4 position for both substrates and inhibitors. For group II and group III caspases, a tryptophan rests at the bottom of the S4 pocket. For group I caspases, this same residue is a smaller hydrophobic amino acid (e.g. Val or Ile, including Val348 in caspase-1). The binding of Ac-DEVD-CHO to Csp1 produces fewer total hydrogen bonds with S4 residues relative to the other caspases studied and this allows for a wider S4 pocket. The combination of fewer hydrogen bonds along with a neutral electrostatic potential in this portion of the binding site suggests that group I caspases could accommodate larger hydrophobic groups at S4, thereby, providing an avenue to gain selectivity in the design of inhibitors. The combinatorial tetrapeptide substrate study showed that WEHD was the optimal substrate for caspase-1 and was also a good substrate for the other group I caspases, caspase-4, and caspase-5 [N. A. Thornberry, J. Biol. Chem., 272, pp. 17907-17911 (1997)]. However, leucine was roughly equal to tryptophan at P4 as a substrate for these two caspases. Group II caspases were shown to have a strong preference for substrates with aspartic acid at P4. As alluded to above, the replacement of hydrophobic residues in caspase-3 by charged or hydrophilic residues in caspase-7 might allow these two seemingly redundant caspases to act on different substrates in different cell types or cellular compartments [J. M. Chandler et al., J. Biol. Chem., 273, 10815-10818 (1998); T. Machleidt, Federation of European Biochemical Studies, 436, pp. 51-54 (1998)] even though their in vitro substrate preferences are identical. Group III caspases prefer either valine or leucine at the P4 position. Interestingly, in terms of inhibitors, AcWEHD-CHO showed the most potent inhibition against caspase-1 suggesting that the valine at the bottom of the S4 pocket leaves enough room for the P4 tryptophan to be accommodated [M. Garcia-Calvo et al., J. Biol. Chem., 273, pp. 32608-32613 (1998)]. Based on the sequence alignment in FIG. 3, caspase-4 and caspase-5 would be expected to possess an isoleucine instead of valine at the same position at the bottom of the S4 pocket. This one extra methyl group still allows the P4 tryptophan from AcWEHD-CHO to fit, but not quite as well (caspase-1, Ki=0.056 nM; Caspase-4, Ki=97 nM; Caspase-5, Ki=43 riM). ACWEHD-CHO was a poor inhibitor of all other group II and group III caspases except caspase-8 (Ki=21 nM). Although caspase-8 has a large trytophan residue at the bottom of the S4 pocket, this portion of the binding site is fairly wide and can accommodate the P4 group of AcWEHD-CHO. Similar trends are observed for inhibitors containing tyrosine or benzyloxycarbonyl groups at P4. Additionally, the first selective Csp1 inhibitor to reach clinical trials, VX-740, possesses an isoquinoline group at P4. The X-ray crystallographic structure of this inhibitor clearly shows the P4 isoquinoline fitting snugly in the S4 pocket (manuscript in preparation). Thus, although it is possible to design selective Csp1 inhibitors by incorporation of large hydrophobic groups at P4, it is not clear that one can take advantage of the P4 position to design selective inhibitors of other caspases.

### EXAMPLE 5

### Use of Caspase-7 Coordinates for Inhibitor Design

The coordinates in FIG. 7 are used to design compounds, including inhibitory compounds, that associate with caspase-7 or homologues of caspase-7. This process may be aided by using a machine-readable data storage medium encoded with a set of machine-readable data storage medium encoded with a set of machine-executable instructions, wherein the recorded instructions are capable of displaying a three-dimensional representation of the caspase-7 complex or a portion thereof. The graphical representation is used according to the methods described herein to design compounds, including inhibitory compounds, that bind to caspase-7. Such compounds may associate with caspase-7 at all or part of the active site.

While we have described a number of embodiments of this invention, it is apparent that our basic examples may be altered to provide other embodiments which utilize the products and processes of this invention. Therefore, it will be appreciated that the scope of this invention is to be defined by the appended claims rather than by the specific embodiments which have been represented by way of example.

We claim:

1. A molecule or molecular complex comprising all or any part of a binding pocket defined by structure coordinates of caspase-7 amino acids 234, 235, 237, 276, 278, 281, and 284 according to FIG. 7, or a homologue of said molecule or molecular complex, wherein said homologue comprises a binding pocket that has a root mean square deviation from the backbone atoms of said amino acids of not more than 1.5 Å, and wherein said molecule or molecular complex has a S4 binding region that is more hydrophilic than the S4 binding region of caspase-3.

2. The molecule or molecular complex according to claim 1, wherein said homologue comprises a binding pocket that has a root mean square deviation from the backbone atoms of said amino acids of not more than 1.0 Å.

3. The molecule or molecular complex according to claim 1, comprising all or any part of a binding pocket defined by structure coordinates of caspase-7 amino acids 85, 86, 87, 88, 144, 145, 184, 186, 191, 223, 230, 231, 232, 233, 234, 235, 237, 240, 276, 278, 281, 282, and 284 according to FIG. 7, or a homologue of said molecule or molecular complex, wherein said homologue comprises a binding pocket that has a root mean square deviation from the backbone atoms of said amino acids of not more than 1.5 Å, and wherein said molecule or molecular complex has a S4 binding region that is more hydrophilic than the S4 binding region of caspase-3.

4. The molecule or molecular complex according to claim 3, wherein said homologue comprises a binding pocket that has a root mean square deviation from the backbone atoms of said amino acids of not more than 1.0 Å.

5. The molecule or molecular complex according to claim 3, wherein said molecule or molecular complex is defined by the structure coordinates according to FIG. 7, or a homologue thereof, wherein said homologue has a root mean square deviation from the conserved backbone atoms of said amino acids of not more than 1.5 Å, and wherein said molecule or molecular complex has a S4 binding region that is more hydrophilic than the S4 binding region of caspase-3.

6. The molecule or molecular complex according to claim 5, wherein said homologue comprises a binding pocket that has a root mean square deviation from the backbone atoms of said amino acids of not more than 1.0 Å.

7. The molecule or molecular complex according to claim 1, wherein said molecule or molecular complex comprises the structure coordinates of caspase-7 amino acids 58-302 according to FIG. 7.

8. A crystallizable composition comprising a caspase-7 or homologue thereof complexed with Ac-Asp-Glu-Val-Asp-CHO.

9. The crystallizable composition according to claim 8, wherein the caspase-7 has amino acids 1-303.

10. A crystal comprising a caspase-7 or homologue thereof complexed with Ac-Asp-Glu-Val-Asp-CHO.

11. The crystal according to claim 10, wherein the caspase-7 has amino acids 1-303.

12. A machine-readable data storage medium, comprising a data storage material encoded with machine readable data, wherein said data is defined by the all or a portion of the structure coordinates of a molecule or molecular complex according to FIG. 7, or a homologue of said molecule or molecular complex, wherein said homologue comprises backbone atoms that have a root mean square deviation from the backbone atoms of the complex of not more than 1.5 Å, and wherein said molecule or molecular complex has a S4 binding region that is more hydrophilic than the S4 binding region of caspase-3.

13. The machine-readable data storage medium according to claim 12, wherein said data is defined by all or a portion of the structure coordinates of caspase-7 amino acids 58-302 according to FIG. 7, or a homologue of said caspase-7, said homologue having a root mean square deviation from the backbone atoms of said amino acids of not more than 1.5 Å.

14. A machine-readable data storage medium comprising a data storage material encoded with a first set of machine readable data which, when combined with a second set of machine readable data using a machine programmed with instructions for using said first set of data and said second set of data, can determine at least a portion of the structure coordinates corresponding to the second set of machine readable data, wherein: said first set of data and said second set of data comprises a Fourier transform of at least a portion of the structure coordinates of caspase-7 according to FIG. 7; said second set of data comprises an X-ray diffraction pattern of a molecule or molecular complex of unknown structure.

15. A computer for producing a three-dimensional representation of:
a. a molecule or molecular complex comprising all or any part of a binding pocket defined by structure coordinates of caspase-7 amino acids 234, 235, 237, 276, 278, 281, and 284 according to FIG. 7; or
b. a homologue of said molecule or molecular complex, wherein said homologue comprises a binding pocket that has a root mean square deviation from the backbone atoms of said amino acids of not more than 1.5 Å, and wherein said molecule or molecular complex has a S4 binding region that is more hydrophilic than the S4 binding region of caspase-3, wherein said computer comprises:
i. a machine-readable data storage medium comprising a data storage material encoded with machine-readable data, wherein said data comprises the structure coordinates of caspase-7 amino acids 234, 235, 237, 276, 278, 281, and 284 according to FIG. 7; and
ii. instructions for processing said machine-readable data into said three-dimensional representation.

16. The computer according to claim 15, wherein said homologue comprises a binding pocket that has a root mean square deviation from the backbone atoms of said amino acids of not more than 1.0 Å.

17. The computer according to claim 15, wherein:
a. the molecule or molecular complex comprises all or any part of a binding pocket defined by structure coordinates of caspase-7 amino acids 85, 86, 87, 88, 144, 145, 184, 186, 191, 223, 230, 231, 232, 233, 234, 235, 237, 240, 276, 278, 281, 282, and 284 according to FIG. 7; or
b. a homologue of said molecule or molecular complex, wherein said homologue comprises a binding pocket that has a root mean square deviation from the backbone atoms of said amino acids of not more than 1.5 Å, and wherein said molecule or molecular complex has a S4 binding region that is more hydrophilic than the S4 binding region of caspase-3 wherein said computer comprises:
i. a machine-readable data storage medium comprising a data storage material encoded with machine-readable data, wherein said data comprises the structure coordinates of caspase-7 amino acids 85, 86, 87, 88, 144, 145, 184, 186, 191, 223, 230, 231, 232, 233, 234, 235, 237, 240, 276, 278, 281, 282, and 284 according to FIG. 7; and
ii. instructions for processing said machine-readable data into said three-dimensional representation.

18. The computer according to claim 17, wherein said homologue comprises a binding pocket that has a root mean square deviation from the backbone atoms of said amino acids of not more than 1.0 Å.

19. The computer according to claim 17, wherein:
a. said molecule or molecular complex comprises structure coordinates according to FIG. 7; or
b. a homologue of said molecule or molecular complex, wherein said homologue comprises a binding pocket that has a root mean square deviation from the backbone atoms of said amino acids of not more than 1.5 Å, and wherein said molecule or molecular complex has a S4 binding region that is more hydrophilic than the S4 binding region of caspase-3 wherein said computer comprises:
i. a machine-readable data storage medium comprising a data storage material encoded with machine-readable data, wherein said data comprises the structure coordinates according to FIG. 7;
ii. instructions for processing said machine-readable data into said three-dimensional representation.

20. The computer according to claim 23, wherein said homologue comprises a binding pocket that has a root mean square deviation from the backbone atoms of said amino acids of not more than 1.0 Å.

21. The computer according to claim 17, wherein:
a. said molecule or molecular complex comprises all or any part of a binding pocket defined by structure coordinates of caspase-7 amino acids 58-302 according to FIG. 7; wherein said computer comprises:
i. a machine-readable data storage medium comprising a data storage material encoded with machine-readable data, wherein said data comprises the structure coordinates of caspase-7 amino acids 58-302 according to FIG. 7;
ii. instructions for processing said machine-readable data into said three-dimensional representation.

22. The computer according to any one of claims 15-21, further comprising a display for displaying said three-dimensional representation.

23. A computer for determining at least a portion of the structure coordinates corresponding to X-ray diffraction data obtained from a molecule or molecular complex, wherein said computer comprises:
a. a machine-readable data storage medium comprising a data storage material encoded with machine-readable data, wherein said data comprises at least a portion of the structure coordinates of caspase-7 according to FIG. 7;
b. a machine-readable data storage medium comprising a data storage material encoded with machine-readable data, wherein said data comprises X-ray diffraction data obtained from said molecule or molecular complex; and
c. instructions for performing a Fourier transform of the machine readable data of (a) and for processing said machine readable data of (b) into structure coordinates.

24. The computer according to claim 23, further comprising a display for displaying said three-dimensional representation.

25. A method for evaluating the ability of a chemical entity to associate with a molecule or molecular complex according to any one of claims 1-7 comprising the steps of:
a. employing computational means to perform a fitting operation between the chemical entity and a binding pocket of the molecule or molecular complex; and
b. analyzing the results of said fitting operation to quantify the association between the chemical entity and the binding pocket.

26. A method for identifying a potential agonist or antagonist of a molecule or molecular complex according to any one of claims 1-7 comprising the steps of:
a. generating a three-dimensional structure of the molecule or molecular complex;
b. employing said three-dimensional structure to design or select said potential agonist or antagonist;
c. providing said potential agonist or antagonist; and
d. contacting said potential agonist or antagonist to interact with said molecule.

27. A method of utilizing molecular replacement to obtain structural information about a molecule or a molecular complex of unknown structure by using the structure coordinates set forth in FIG. 7, comprising the steps of:
a. crystallizing said molecule or molecular complex;
b. generating a X-ray diffraction pattern from said crystallized molecule or molecular complex; and
c. applying at least a portion of the structure coordinates set forth in FIG. 7 to said X-ray diffraction pattern to generate a three-dimensional electron density map of at least a portion of the molecule or molecular complex whose structure is unknown.

28. The method according to claim 27, wherein the molecule or molecular complex of unknown structure comprises a polypeptide selected from a caspase-7 subunit or a capase-7 homologue.
